(19) United States
(12) Patent Application Publication
(10) Pub. No.: US 2022/0228221 A1
(43) Pub. Date: Jul. 21, 2022

(54) Diagnostics and Treatments Based Upon Molecular Characterization of Colorectal Cancer

(71) Applicant: The Board of Trustees of the Leland Stanford Junior University, Stanford, CA (US)

(72) Inventor: Christina Curtis, Stanford, CA (US)

(73) Assignee: The Board of Trustees of the Leland Stanford Junior University, Stanford, CA (US)

(21) Appl. No.: 17/596,821

(22) PCT Filed: Jun. 17, 2020
(86) PCT No.: PCT/US2020/038238

§ 371 Date: Dec. 17, 2021

Related U.S. Application Data
(60) Provisional application No. 62/862,609, filed on Jun. 17, 2019.

(51) Int. Cl. C12Q 1/6886

(52) U.S. Cl. CPC C12Q 1/6886; C12Q 2600/112; C12Q 2600/106; C12Q 2600/156

(57) ABSTRACT

Diagnostics and treatments based on a colorectal cancer's genetic aberrations are provided. Combinations of various genes harboring genetic aberrations are used to molecularly subtype patients and in some instances to determine a colorectal cancer's metastatic potential. In some instances, a of colorectal cancer having a particular set of genes harboring genetic aberrations is treated with a targeted therapy specific targeting the oncogenic genes.

### CROSS REFERENCE TO RELATED APPLICATIONS

This application claims priority to U.S. Provisional Application Ser. No. 62/862,609, entitled “Methods of Treatments Based Upon Molecular Characterization of Colorectal Cancer” by Christina Curtis, filed Jun. 17, 2019, the disclosure of which is incorporated herein by reference in its entirety.

### TECHNICAL FIELD

The invention is generally directed to diagnostics and treatments based upon molecular characterization of an individual's colorectal cancer, and more specifically to treatments based upon molecular diagnostics indicative of risk of metastasis in colorectal cancer.

### BACKGROUND

Metastasis is the primary cause of death in cancer patients, but the timing and molecular determinants of this process are largely uncharacterized, hindering treatment and prevention efforts. In particular, when and how metastatic competence is specified is of clinical significance. The prevailing linear progression model posits that metastatic capacity is acquired late following the gradual accumulation of somatic alterations, such that only a subset of cells evolve the capacity to disseminate and seed metastases. At odds with this view, gene expression signatures from primary tumors are (partially) predictive of distant recurrence indicating that metastatic cells constitute a dominant subpopulation in primary tumor. However, the timing of metastatic dissemination has not been evaluated in human cancers due to the challenge in obtaining paired primary tumors and distant metastases and the limitations of phylogenetic approaches.

### SUMMARY

Various embodiments are directed to diagnostics and treatments of colorectal cancer. In various embodiments, a biopsy of an individual is acquired and assessed for genetic aberrations in particular sets of genes that confer a pathogenic effect. In various embodiments, treatments are performed based on the genetic aberrations detected.

In an embodiment, a method is for determining an individual's risk for colorectal cancer. The method obtains a biopsy of an individual having colorectal cancer. The method detects that the biopsy includes genetic aberrations occurring within the genes PTPRT, TCF7L2, AMER1 APC, KRAS, TP53, or SMAD4. The method determines that each gene of one of the following combinations of gene sets exhibits a genetic abnormality that confers a pathogenic effect on gene function:
PTPRT and one of: APC, KRAS, TP53 or SMAD4,
PTPRT and APC and KRAS,
PRPRT and APC and TP53,
PTPRT and TP53 and KRAS,
PTPRT and TP53 and SMAD4,
PTPRT and TP53 and KRAS and SMAD4,
AMER1 and one of: APC, KRAS or TP53,
AMER1 and APC and KRAS,
AMER1 and APC and TP5,
TCF7L2 and one of: APC or TP53, or
TCF7L2 and APC and TP53.

In another embodiment, the method further administers to the individual a treatment based upon that each gene of a said gene set combination exhibits a genetic abnormality, which is further based upon the clinical stage of cancer progression.

In yet another embodiment, the clinical stage is classified as Stage 0 and the treatment includes a local excision or a polypectomy and prolonged monitoring after the local excision or the polypectomy.

In a further embodiment, the clinical stage is classified as Stage I and the treatment includes a surgical resection and prolonged monitoring after the surgical resection.

In still yet another embodiment, the clinical stage is classified as Stage II and the treatment includes a surgical resection and an adjuvant chemotherapy.

In yet a further embodiment, the clinical stage is classified as Stage II and the treatment includes a surgical resection and a targeted therapy.

In an even further embodiment, the clinical stage is classified as Stage III and the treatment includes a surgical resection with a prolonged adjuvant chemotherapy.

In yet an even further embodiment, the clinical stage is classified as Stage III and the treatment includes a surgical resection and an adjuvant chemotherapy typical for metastatic colorectal cancer.

In still yet an even further embodiment, the clinical stage is classified as Stage III and the treatment includes a surgical resection and a targeted therapy.

In still yet an even further embodiment, the clinical stage is classified as Stage IV and the treatment includes an adjuvant chemotherapy and a targeted therapy.

In still yet an even further embodiment, the biopsy is a tumor biopsy or liquid biopsy.

In still yet an even further embodiment, the biopsy is derived from a primary tumor, a nodal tumor, or a distal tumor.

In still yet an even further embodiment, the genetic aberrations detected are single nucleotide variants, insertions, deletions, or copy number alterations (CNAs).

In still yet an even further embodiment, the determination that each gene of one of the following combinations of gene sets exhibits a genetic abnormality include analysis of at least one of: genomic sequence mutation, copy number aberration, DNA methylation, RNA transcript expression level, or protein expression level.

In still yet an even further embodiment, the genetic aberration is detected by an assay selected from the group consisting of: nucleic acid hybridization, nucleic acid proliferation, and nucleic acid sequencing.

In still yet an even further embodiment, the pathogenic effect on the gene function is known to confer an oncogenic effect.

In still yet an even further embodiment, the pathogenic effect on the gene function is assumed to confer an oncogenic effect.

In still yet an even further embodiment, the pathogenic effect on the gene function is determined to likely confer an oncogenic effect.

In still yet an even further embodiment, the pathogenic effect on the gene function is determined by a computational program.

In still yet an even further embodiment, the pathogenic effect on the gene function is determined by a biological assay.

In an embodiment, a method is for screening an individual for colorectal cancer. The method obtains a liquid biopsy of an individual. The method detects colorectal cancer in the liquid biopsy. The method detects that the colorectal cancer includes genetic aberrations occurring in the genes PTPRT, TCF7L2, AMER1 APC, KRAS, TP53, or SMAD4. The method determines that each gene of one of the following combinations of gene sets exhibits a genetic abnormality that confers a pathogenic effect on the gene function:
PTPRT and one of: APC, KRAS, TP53 or SMAD4,
PTPRT and APC and KRAS,
PRPRT and APC and TP53,
PTPRT and TP53 and KRAS,
PTPRT and TP53 and SMAD4,
PTPRT and TP53 and KRAS and SMAD4,
AMER1 and one of: APC, KRAS or TP53,
AMER1 and APC and KRAS,
AMER1 and APC and TP5,
TCF7L2 and one of: APC or TP53, or
TCF7L2 and APC and TP53.

In another embodiment, the colorectal cancer is detected in the liquid biopsy by detecting the presence of circulating tumor DNA or cancerous cells.

In yet another embodiment, the method further confirms that the individual has colorectal cancer by extracting and examining a lymph node biopsy.

In a further embodiment, the method further confirms that the individual has colorectal cancer by capturing a medical image the individual.

In still yet another embodiment, the medical image is captured via endoscopy, X-ray, magnetic resonance imaging (MRI), computed tomography (CT), ultrasound, and positron emission tomography (PET).

In yet a further embodiment, the method further administers to the individual a treatment based upon that each gene of a said gene set combination exhibits a genetic abnormality, which is further based upon the clinical stage of cancer progression.

In an even further embodiment, the clinical stage is classified as Stage 0 and the treatment includes a local excision or a polypectomy and prolonged monitoring after the local excision or the polypectomy.

In yet an even further embodiment, the clinical stage is classified as Stage I and the treatment includes a surgical resection and prolonged monitoring after the surgical resection.

In still yet an even further embodiment, the clinical stage is classified as Stage II and the treatment includes a surgical resection and an adjuvant chemotherapy.

In still yet an even further embodiment, the clinical stage is classified as Stage II and the treatment includes a surgical resection and a targeted therapy.

In still yet an even further embodiment, the clinical stage is classified as Stage III and the treatment includes a surgical resection with a prolonged adjuvant chemotherapy.

In still yet an even further embodiment, the clinical stage is classified as Stage III and the treatment includes a surgical resection and an adjuvant chemotherapy typical for metastatic colorectal cancer.

In still yet an even further embodiment, the clinical stage is classified as Stage III and the treatment includes a surgical resection and a targeted therapy.

In still yet an even further embodiment, the clinical stage is classified as Stage IV and the treatment includes an adjuvant chemotherapy and a targeted therapy.

In still yet an even further embodiment, the genetic aberrations detected are single nucleotide variants, insertions, deletions, or copy number alterations (CNAs).

In still yet an even further embodiment, the determination that each gene of one of the following combinations of gene sets exhibits a genetic abnormality include analysis of at least one of: genomic sequence mutation, copy number aberration, DNA methylation, RNA transcript expression level, or protein expression level.

In still yet an even further embodiment, the genetic aberrations include analysis of at least one of: genomic sequence mutation, copy number aberration, DNA methylation, RNA transcript expression level, or protein expression level.

In still yet an even further embodiment, the genetic aberration is detected by an assay selected from the group consisting of: nucleic acid hybridization, nucleic acid proliferation, and nucleic acid sequencing.

In still yet an even further embodiment, the pathogenic effect on the gene function is known to confer an oncogenic effect.

In still yet an even further embodiment, the pathogenic effect on the gene function is assumed to confer an oncogenic effect.

In still yet an even further embodiment, the pathogenic effect on the gene function is determined to likely confer an oncogenic effect.

In still yet an even further embodiment, the pathogenic effect on the gene function is determined by a computational program.

In still yet an even further embodiment, the pathogenic effect on the gene function is determined by a biological assay.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow diagram of a method to treat colorectal cancer based upon a molecular classification indicative of metastatic ability in accordance with various embodiments of the invention.

FIG. 2 provides a flow diagram of a method to perform early diagnostics for colorectal cancer and applications thereof utilizing a liquid biopsy in accordance with various embodiments of the invention.

FIG. 3 provides a table of categorizing colorectal cancers, utilized in accordance with various embodiments.

FIG. 4A provides a schematic of the metastatic colorectal cancer (mCRC) cohort analyzed, utilized in accordance with various embodiments.

FIG. 4B provides a schema of computational modeling to infer timing of metastasis, utilized in accordance with various embodiments.

FIG. 5 provides a flow chart of a method to infer timing of metastasis from harvested primary tumor tissue, metastatic tissue, and normal tissue, utilized in accordance with various embodiments.

FIG. 6 provides a schema of phylogenetic reconstruction of mCRC and a data chart depicting the cancer cell fraction (CCF), utilized in accordance with various embodiments.

FIG. 7 provides tumor cell purity estimates in each tumor sample obtained from the brain metastasis and liver metastasis cohorts, utilized in accordance with various embodiments.

FIG. 8 provides results of multi-region sequencing to identify clonal SNVs within tumors, utilized in accordance with various embodiments.

FIG. 9 provides a schema of the CRC validation cohort, utilized in accordance with various embodiments.

FIG. 10 provides data of genes harboring somatic SNVs in from a selection of patients within the brain metastasis and liver metastasis cohorts, utilized in accordance with various embodiments.

FIG. 11 provides a data chart depicting high frequency somatic SNVs existed in both the primary and metastatic tumors, utilized in accordance with various embodiments.

FIGS. 12 and 13 provide data charts depicting cancer drivers (especially CRC drivers) aberrations are shared in both primary and metastatic tumors, utilized in accordance with various embodiments.

FIG. 14 provides copy number alterations in the primary tumor (P), lymph node biopsy (LN), and metastatic tissue (BM for brain; LI for liver; LU for lungs), utilized in accordance with various embodiments.

FIGS. 15 and 16 provide data charts depicting shared and private somatic SNVs in the cancer cell fraction of biopsies, utilized in accordance with various embodiments.

FIG. 17 provides data charts depicting the type of aberration that are shared or private between primary tumor and brain metastatic tissues, utilized in accordance with various embodiments.

FIGS. 18 provides timelines depicting metastatic occurrence of select patients, utilized in accordance with various embodiments.

FIGS. 19 and 20 provide clinical histories and intra-tumor heterogeneity (ITH) in paired primary and metastatic tumors of select patients, utilized in accordance with various embodiments.

FIG. 21 provides FST based quantification of genetic divergence and Ki67 proliferative indices in metastatic CRCs.

FIG. 22 provides phylogenies of CRC metastasis, utilized in accordance with various embodiments.

FIGS. 23 to 27 provide density plots of CCF estimates in in paired primary and metastatic tumors of select patients, utilized in paired primary and metastatic tumors of select patients, utilized in accordance with various embodiments.

FIG. 28 provides a schema depicting the distinction between the time of primary and metastatic divergence and the actual time of dissemination, utilized in accordance with various embodiments.

FIGS. 29 to 31 each provides a schema depicting a computational model to simulate spatial growth, progression and lineage relationships for neutral and selected subclones, utilized in accordance with various embodiments.

FIGS. 32 to 34 each provides results of the computational model to simulate spatial growth, progression and lineage relationships for neutral and selected subclones, utilized in accordance with various embodiments.

FIG. 35 provides a schematic of Spatial Computational Inference of MEtastatic Timing, utilized in accordance with various embodiments.

FIG. 36 provides results of the Spatial Computational Inference of MEtastatic Timing on synthetic data, utilized in accordance with various embodiments.

FIGS. 37 to 40 each provides mutation rate and primary carcinoma in size for select patients within the brain and liver metastasis cohorts, utilized in accordance with various embodiments.

FIGS. 41 and 42 each provides results of the Spatial Computational Inference of MEtastatic Timing on patient data, utilized in accordance with various embodiments.

FIGS. 43 to 45 each provide data tables depicting the enrichment of canonical driver gene modules in metastatic verses early stage CRCs, utilized in accordance with various embodiments.

FIG. 46 provides a schema for explaining the metastatic seeding that is occurring in the primary tumor, utilized in accordance with various embodiments.

FIGS. 47 to 51 each provide data plots depicting co-occurrence of PTPRT, TCF7L2, and AMER1 co-occur with APC, KRAS, TP53, and/or SMAD4, utilized in accordance with various embodiments.

FIGS. 52 to 55 each provide data tables depicting exemplary colorectal patients that each experienced a metastatic event, utilized in accordance with various embodiments.

FIG. 56 provides a table with potential gene combinatorial that may confer aggressiveness and metastatic potential when each gene harbors a genetic aberration, utilized in accordance with various embodiments.

FIGS. 57 to 59 provide are lollipop plots that show a number of known genetic aberrations that occur in PTPRT, TCF7L2, and AMER1 in various cancers, utilized in accordance with various embodiments.

### DETAILED DESCRIPTION

Turning now to the drawings and data, methods of detecting, diagnosing and treating colorectal cancer based upon the cancer's molecular pathology, in accordance with various embodiments, are provided. Numerous embodiments are directed towards genetically evaluating a tumor biopsy of a patient that has been diagnosed with colorectal cancer. In some embodiments, an individual being assessed has not yet been diagnosed with cancer. In some embodiments, presence of colorectal cancer is determined utilizing a liquid biopsy of plasma derived cell free circulating tumor DNA (ctDNA) and/or circulating tumor cells (CTCs).

Many embodiments are directed to determining a colorectal cancer's potential for metastasis based on its molecular character and then treating that neoplasm accordingly. In some embodiments, a colorectal cancer is evaluated utilizing a tumor biopsy (e.g., primary tumor and/or lymph node biopsy). In some embodiments, a colorectal cancer is evaluated utilizing a liquid biopsy of plasma derived ctDNA and/or CTCs. In some embodiments, nucleic acid genetic data of various genes provide an indication of colorectal cancer molecular pathology and thus provide a means of determining appropriate treatment. In some embodiments, metastatic potential is determined early in the pathology of disease (e.g., before metastasis is detected).

In accordance with multiple embodiments, colorectal cancers exhibiting particular molecular pathologies indicating high aggression and high potential for metastasis are treated aggressively with an appropriate therapy, such as chemotherapy, prolonged treatment, immunotherapy, and/or a targeted therapy. A targeted therapy, in accordance with various embodiments, is a molecularly targeted therapy directed against specific molecular aberrations. Furthermore, in some embodiments, individuals with cancer that has been determined to have high potential for metastasis are closely and repeatedly monitored to detect minimal residual disease (e.g., by imaging modalities or via non-invasive liquid biopsy techniques to profile ctDNA and/or CTCs). In some embodiments, individuals with cancer that have high potential for metastasis are closely and repeatedly monitored for an extended period of time after an initial treatment, and in some cases individuals are continually monitored even when the initial treatment reduces the cancer to undetectable levels. In some embodiments, early stage colorectal cancers exhibiting a molecular pathology indicative of low aggression and recurrence are treated appropriately, which may be include no chemotherapy or less aggressive chemotherapy.

In some embodiments, cancers having a particular molecular pathology are treated with a targeted therapy that is directed at the genes that classify the molecular pathology (e.g., tumors with mutations in PTPRT gene can be treated with STAT3 inhibitors). In some embodiments, biomarkers are used to stratify patients, which may depend on cancer stage. For example, in some embodiments, biomarkers are particularly relevant for stage II colon cancer patients, in which the benefit of standard chemotherapy remains unclear in this population due to variable success and relapse. For these stage II patients, various embodiments are directed towards examining the cancer derived genetic material for molecular biomarkers to determine their risk of relapse and thus stratify these patients accordingly.

A number of embodiments are directed to determining the molecular pathology of a patient's tumor and/or ctDNA and/or CTCs. In some embodiments, an individual's DNA and/or RNA is extracted from a biopsy to assess the genetic aberrations present, which can be used to classify an individual's cancer. Genetic aberrations include (but are not limited to) single nucleotide variants, insertions, deletions, and copy number alterations (CNAs). CNAs are to be understood as amplification (e.g., duplication) and/or reduction (e.g., deletion) of a set of genomic loci within the genome. In some embodiments, a cancer is classified by genetic aberrations in a combinatorial set of genes, which can be referred to as a set of molecular drivers (i.e., genes classified to be at least partially pathogenic in tumorigenesis).

Based on recent discoveries, the connection between the molecular pathology and cancer progression, including the potential for metastasis at an early stage of tumorigenesis, is now appreciated, indicating courses of treatment and surveillance. Accordingly, embodiments are directed to classifying colorectal cancer into a pathological subgroup to determine a treatment regime that is well-suited for a particular colorectal cancer.

### Treatment of Colorectal Cancer Determined by Molecular Characterization

A number of embodiments are directed to classifying a colorectal cancer. In several embodiments, a colorectal cancer is classified based on its DNA and/or transcript expression, which is used to identify somatic genetic aberrations. Particular combinations of genes having genetic alterations, in accordance with several embodiments, indicate the aggressiveness and risk of metastasis. In some embodiments, risk of metastasis is determined early, utilizing an early biopsy of the primary tumor and before metastasis is presented. Accordingly, in various embodiments, tumor and liquid biopsies are utilized to identify combinatorial sets of genetic drivers that indicate metastatic potential and likely site of metastasis. Based on a classification of metastatic potential, a number of embodiments determine a course of treatment for a colorectal cancer, which may include measures to prevent and target metastases.

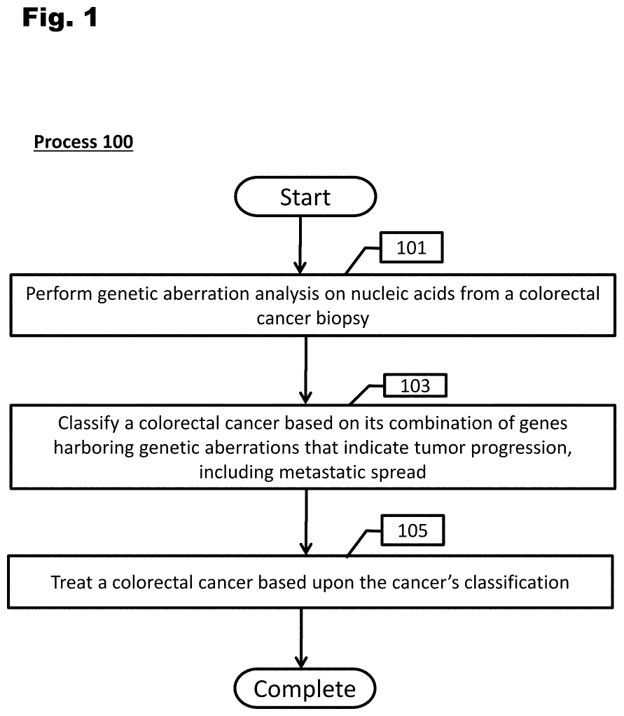

Provided in FIG. 1 is a method to classify a colorectal cancer according to a particular combination of genes harboring genetic aberrations, which is indicative of metastatic potential, and to treat the cancer accordingly. Process 100 begins with performing (101) genetic aberration analysis on nucleic acids from a colorectal cancer biopsy. In several embodiments, DNA and/or RNA transcripts are extracted from an individual having colorectal cancer and processed for analysis. In some embodiments, DNA and/or RNA transcripts are extracted from a tumor and/or liquid biopsy. In some embodiments, DNA and/or RNA transcripts are extracted any time prior to detection of metastasis. In some embodiments, DNA and/or RNA transcripts are extracted early in tumor progression. In some embodiments, DNA and/or RNA is extracted prior to detection of cancer, upon first biopsy extraction, at diagnosis, at the time of surgery, or after an initial treatment.

Genetic aberrations can be detected by a number of methods. In some embodiments, DNA or RNA of a cancer is extracted from an individual and processed to detect genetic aberrations. In some embodiments, DNA is extracted from a biopsy to detect somatic mutations and copy number variations. In various embodiments, RNA is extracted and processed to detect expression levels of a number of genes, which can be utilized to determine alterations in gene expression. In some embodiments, proteins are either extracted and/or examined in fixed tissue to determine protein expression levels and or expression of proteins having particular mutations.

Biomolecules (including nucleic acids and proteins) can be extracted from a cancer biopsy by a number of methodologies, as understood by practitioners in the field. Once extracted, biomolecules can be processed and prepared for detection. Methods of detection include (but are not limited to) hybridization techniques (e.g., in situ hybridization (ISH)), nucleic acid proliferation techniques (e.g., PCR), immunodetection, chromatin immunoprecipitation (ChIP), sequencing (e.g., exome sequencing, whole genome sequencing, targeted sequencing RNA sequencing), DNA methylation (measured via bisulfite sequencing or array based profiling), protein detection (e.g., Western blot, ELISA, histology). It is noted, in some instances, various techniques can be combined such as (for example) DNA methylation analysis along with sequencing.

As depicted, process 100 also classifies (103) a colorectal cancer based on its combination of genes harboring genetic aberrations that indicate tumor progression, including metastatic spread. In several embodiments, a colorectal cancer is classified by genetic aberrations in a set of genetic drivers (i.e., a combinatorial set of genes having genetic aberrations that promote metastasis). Various combinations of genes having genetic aberrations have been found to dictate metastasis. Accordingly, specific combinations of genes harboring aberrations indicate a colorectal cancer is or will be aggressive and have a high risk of metastasis, while the lack of mutations in specific genes in combination indicate a colorectal cancer will be less aggressive, unlikely to metastasize. In many embodiments, a colorectal cancer is examined to determine a collection of genetic aberrations it harbors to classify the cancer. In several embodiments, genomic driver classification is determined by genomic sequence mutations, copy number aberrations, DNA methylation, RNA transcript expression level, protein expression level, or a combination thereof.

In a number of embodiments, specific combinations of genes harboring genetic aberrations were associated with metastatic potential. As detailed in the Exemplary embodiments, it has been found that mutations in driver genes such as adenomatous polyposis coli (APC), KRAS, tumor protein 53 (TP53) or SMAD4, abbreviated A/K/T/S) in combination with aberrations in genes such as protein tyrosine phosphatase receptor type T (PTPRT), transcription factor 7 like 2 (TCF7L2), or APC membrane recruitment protein 1 (AMER1) are indicative of aggressive disease. In particular, the following combinations of genes (when harboring mutations) indicate a high level of aggression and an increased likelihood of metastasis:
PTPRT+[APC or KRAS or TP53 or SMAD4]
PTPRT+[APC and KRAS]
PRPRT+[APC and TP53]
PTPRT+[TP53 and KRAS]
PTPRT+[TP53 and SMAD4]
PTPRT+[TP53 and KRAS and SMAD4]
AMER1+[APC or KRAS or TP53]
AMER1+[APC and KRAS]
AMER1+[APC and TP53]
TCF7L2+[APC or TP53]
TCF7L2+[APC and TP53]

Alterations in the tumor suppressors PTPRT, AMER1, TCF7L2, APC, TP53, and SMAD4 confer loss of function, whereas alteration in the KRAS oncogene confer gain of function. Accordingly, various embodiments utilize loss-of-function mutations within a tumor suppressor gene to indicate a high level of aggression and an increased likelihood of metastasis. Likewise, various embodiments utilize gain-of-function mutations within a tumor suppressor gene to indicate a high level of aggression and an increased likelihood of metastasis. In some embodiments, the oncogenic effect of a particular mutation within a gene is known and utilized to determine its pathogenic effect. In some embodiments, a computational program is utilized to determine a pathogenic effect on gene function, and thus used to determine to likely confer an oncogenic effect. A number of computational programs can be utilized to determine a pathogenic effect, including (but not limited to) VEP (uswest.ensembl.org/Tools/VEP), FATHMM (fathmm.biocompute.org.uk/cancer.html) and CADD (cadd.gs.washington.edu/). In some embodiments, a biological assay is utilized to determine a pathogenic effect on gene function, and thus used to determine to likely confer an oncogenic effect. A number of biological assays could be performed to determine oncogenic effect, including (but not limited to) inducing the mutation within the sequence of the gene in question within an appropriate cellular or animal model and determining the effect of the mutation on oncogenesis.

In some embodiments, mutations within other genes within WNT, TP53, TGFB, EGFR and cellular adhesion pathways are combined to indicate a high level aggression and an increased likelihood of metastasis.

It is now understood that molecular classification is indicative of colorectal tumor progression and metastatic potential. Accordingly, based upon a cancer's classification, a colorectal cancer is treated (105). In various embodiments, a treatment entails chemotherapy, radiotherapy, immunotherapy, hormone therapy, targeted drug therapy, medical surveillance, or any combination thereof. In some embodiments, an individual is treated by medical professional, such as a doctor, nurse, dietician, or similar.

In a number of embodiments, a more aggressive and/or targeted treatment is applied when the cancer harbors mutations that are indicative of a more aggressive cancer with a high likelihood of metastasis. Accordingly, when it is found that a cancer harbors mutations in the genes PTPRT, TCF7L2, and AMER1, and in combination with mutations in the A/K/T/S genes, an appropriate treatment is applied.

The presence of specific combinations of genomic aberrations can be used to determine the cancer's aggressiveness and metastatic potential, and thus an appropriate treatment can be determined and performed. As described herein within the section entitled “Methods of Treatment,” in accordance with various embodiments, an appropriate treatment will often further depend on the stage of colorectal cancer. For example, stage II colorectal cancers are often questioned on whether to pursue an aggressive chemotherapy. In a number of embodiments, a stage II colorectal cancer having an aggressive genotype is treated with a chemotherapeutic agent.

While specific examples of processes for molecularly classifying and treating a colorectal cancer are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for molecularly classifying and treating appropriate to the requirements of a given application can be utilized in accordance with various embodiments of the invention.

### Early Detection of Colorectal Cancer

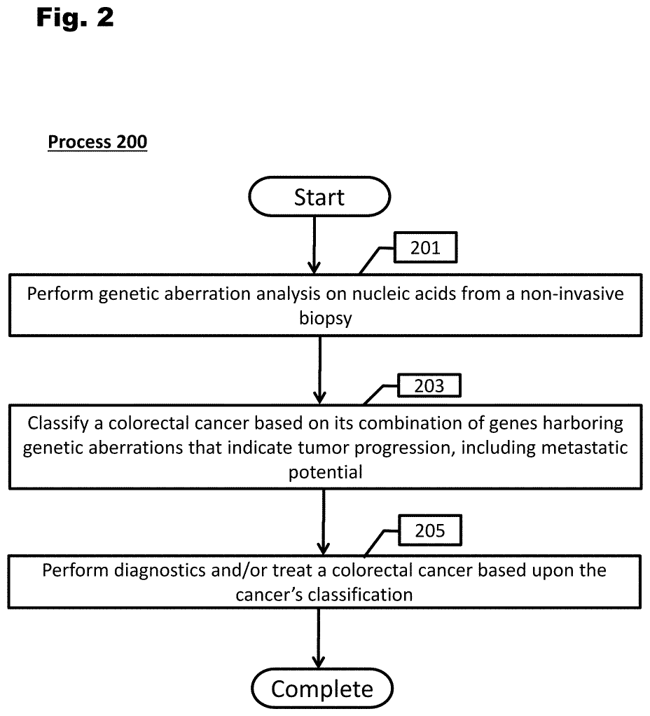

Provided in FIG. 2 is an early detection method such that earlier diagnostics and/or treatments can be performed on a colorectal cancer. In several embodiments, a colorectal cancer will be further classified according to the combination of genes harboring genetic aberrations. Classification of a colorectal cancer is indicative of which diagnostics to perform and which treatments would confer benefit. Process 200 can begin with performing (201) genetic aberration analysis on nucleic acids from a non-invasive biopsy. In some embodiments, ctDNA and/or CTCs are extracted from plasma, blood, lymph, and/or other appropriate bodily fluid. In some embodiments, DNA and/or RNA transcripts are extracted from CTCs and processed for analysis. In some embodiments, a liquid biopsy is extracted prior to a diagnosis or an indication that the individual being analyzed has colorectal cancer. In some embodiments, the genetic aberration analysis is performed as a medical screening, such as (for example) a screening to be performed at routine checkup by a medical professional.

In some embodiments, genetic aberration analysis is performed on an individual with a known risk of developing colorectal cancer, such as those with a familial history of the disorder. In some embodiments, genetic aberration analysis is performed on any individual within the general population. In some embodiments, genetic aberration analysis is performed an individual within a particular age group with higher risk of colorectal cancer, such as individuals between the age of 50 and 75.

Process 200 classifies (203) a colorectal cancer based on its combination of genes harboring genetic aberrations that indicate tumor progression, including metastatic potential. Because neoplasms (especially metastatic tumors) are actively growing and expanding, neoplastic cells are often releasing into the vasculature and/or lymph system. In addition, due to biophysical constraints in their local environment, neoplastic cells are often rupturing, releasing their inner cell contents into the vasculature and/or lymph system. Accordingly, it is possible to detect distal primary tumors and/or metastases from a liquid biopsy. Based on the DNA content from ctDNA and/or colorectal cancer (CRC) cells, in accordance with a number of embodiments, the site of primary tumor and the type of cancer can be determined and thus a colorectal cancer can be identified from a liquid biopsy. Likewise, and in accordance with various embodiments, the genetic information within ctDNA and/or CRC cells can be utilized to classify a colorectal cancer based on the combination of genes harboring genetic aberrations.

Genetic aberrations can be detected by a number of methods. In some embodiments, DNA and/or RNA of a cancer is extracted from an individual and processed to detect genetic aberrations. In a number of embodiments, DNA and/or RNA is extracted from a biopsy to detect somatic mutations and copy number variations.

Biomolecules (especially DNA and/or RNA) can be extracted from a cancer biopsy by a number of methodologies, as understood by practitioners in the field. Once extracted, biomolecules can be processed and prepared for detection. Methods of detection include (but are not limited to) hybridization techniques (e.g., in situ hybridization (ISH)), nucleic acid proliferation techniques (e.g., PCR), immunodetection, chromatin immunoprecipitation (ChIP), sequencing (e.g., exome sequencing, whole genome sequencing, targeted sequencing RNA sequencing), DNA methylation (measured via bisulfite sequencing or array based profiling), protein detection (e.g., Western blot, ELISA, histology). It is noted, in some instances, various techniques can be combined such as (for example) DNA methylation analysis along with sequencing.

In accordance with a variety of embodiments, a colorectal cancer is classified based on its combination of genes harboring genetic aberrations that indicate tumor progression, including metastatic spread. In several embodiments, a colorectal cancer is classified by genetic aberrations in a set of genetic drivers (i.e., a combinatorial set of genes having genetic aberrations that promote metastasis). Various combinations of genes having genetic aberrations have been found to dictate metastasis. Accordingly, specific combinations of genes harboring aberrations indicate a colorectal cancer is or will be aggressive and have a high risk of metastasis, while the lack of mutations in specific genes in combination indicate a colorectal cancer will be less aggressive, unlikely to metastasize. In many embodiments, a colorectal cancer is examined to determine a collection of genetic aberrations it harbors to classify the cancer. In several embodiments, genomic driver classification is determined by genomic mutations, copy number aberrations, DNA methylation, RNA transcript expression, protein expression, or a combination thereof.

In a number of embodiments, specific combinations of genes harboring genetic aberrations were associated with metastatic potential. As detailed in the Exemplary embodiments, it has been found that mutations in driver genes such as adenomatous polyposis coli (APC), KRAS, tumor protein 53 (TP53) or SMAD4, abbreviated A/K/T/S) in combination with aberrations in genes such as protein tyrosine phosphatase receptor type T (PTPRT), transcription factor 7 like 2 (TCF7L2), or APC membrane recruitment protein 1 (AMER1) are indicative of aggressive disease. In particular, the following combinations genes (when harboring mutations) indicate a high level aggression and an increased likelihood of metastasis:
PTPRT+[APC or KRAS or TP53 or SMAD4]
PTPRT+[APC and KRAS]
PRPRT+[APC and TP53]
PTPRT+[TP53 and KRAS]
PTPRT+[TP53 and SMAD4]
PTPRT+[TP53 and KRAS and SMAD4]
AMER1+[APC or KRAS or TP53]
AMER1+[APC and KRAS]
AMER1+[APC and TP53]
TCF7L2+[APC or TP53]
TCF7L2+[APC and TP53]

Alterations in the tumor suppressors PTPRT, AMER1, TCF7L2, APC, TP53, and SMAD4 confer loss of function, whereas alteration in the KRAS oncogene confer gain of function. Accordingly, various embodiments utilize loss-of-function mutations within a tumor suppressor gene to indicate a high level of aggression and an increased likelihood of metastasis. Likewise, various embodiments utilize gain-of-function mutations within a tumor suppressor gene to indicate a high level of aggression and an increased likelihood of metastasis. In some embodiments, the oncogenic effect of a particular mutation within a gene is known and utilized to determine its pathogenic effect. In some embodiments, a computational program is utilized to determine a pathogenic effect on gene function, and thus used to determine to likely confer an oncogenic effect. A number of computational programs can be utilized to determine a pathogenic effect, including (but not limited to) VEP (uswest.ensembl.org/Tools/VEP), FATHMM (fathmm.biocompute.org.uk/cancer.html) and CADD (cadd.gs.washington.edu/). In some embodiments, a biological assay is utilized to determine a pathogenic effect on gene function, and thus used to determine to likely confer an oncogenic effect. A number of biological assays could be performed to determine oncogenic effect, including (but not limited to) inducing the mutation within the sequence of the gene in question within an appropriate cellular or animal model and determining the effect of the mutation on oncogenesis.

In some embodiments, mutations within other genes within WNT, TP53, TGFB, EGFR and cellular adhesion pathways are combined to indicate a high level aggression and an increased likelihood of metastasis.

It is now understood that molecular classification is indicative of colorectal tumor progression and metastatic potential. Accordingly, based upon a cancer's classification, further diagnostics are performed (105) and a colorectal cancer is treated. In a number of embodiments, a diagnostic is a blood test, medical imaging, colonoscopy, physical exam, a biopsy, or any combination thereof. In several embodiments, diagnostics are preformed to determine the particular stage of colorectal cancer. In a number of embodiments, a treatment entails chemotherapy, radiotherapy, immunotherapy, hormone therapy, targeted drug therapy, medical surveillance, or any combination thereof. In some embodiments, an individual is treated by medical professional, such as a doctor, nurse, dietician, or similar.

In a number of embodiments, when an aggressive cancer is indicated, medical imaging, nodal biopsies, and liquid biopsies are performed to identify any possible metastasis. In some embodiments, when signs of metastasis are not present in spite of an indication of an aggressive cancer, routine check-ups are performed to monitor the cancer's progression. Accordingly, when it is found that a cancer harbors mutations in the genes PTPRT, TCF7L2, and AMER1, and in combination with mutations in the A/K/T/S genes, an appropriate diagnostic routine is applied.

Likewise, an appropriate treatment can be determined and performed based on the presence of specific combinations of genomic aberrations can. As described herein within the section entitled “Methods of Treatment,” in accordance with various embodiments, an appropriate treatment will often further depend on the stage of colorectal cancer. For example, stage II colorectal cancers are often questioned on whether to pursue an aggressive chemotherapy. In a number of embodiments, a stage II colorectal cancer having an aggressive genotype is treated with a chemotherapeutic agent.

While specific examples of processes for performing genetic aberration analysis and further diagnostics are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for performing genetic aberration analysis and further diagnostics appropriate to the requirements of a given application can be utilized in accordance with various embodiments of the invention.

### Methods of Detecting Genetic Aberrations

Genetic aberrations can be detected by a number of methods in accordance with various embodiments of the invention, as would be understood by those skilled in the art. In several embodiments, genetic aberrations are alterations in the genetic code that lead to a disruption or gain of gene function. Genetic aberrations include (but are not limited to) single nucleotide variants, insertions, deletions, and copy number alterations (CNAs). CNAs are amplification (e.g., duplication) and/or reduction (e.g., deletion) of a set of genomic loci. Genetic aberrations can result in number alterations in gene and protein expression, including alteration of amino acid code, protein truncations, alteration in expression level, alteration in epigenetic regulation, alteration in gene splicing, and a combination thereof. In sum, a genetic aberration results in an alteration of expression of a gene or its protein, which in turn confers an oncogenic potential.

To determine genetic aberrations, in accordance with a variety of embodiments, biomolecules (e.g., DNA, RNA or protein) are extracted from a tumor or liquid biopsy. Several methods can be used to extract biomolecules from biological sources. Generally, biomolecules are extracted from cells or tissue, then prepped for further analysis. Alternatively, biomolecules can be observed within cells, which are typically fixed and prepped for further analysis. The decision to extract nucleic acids or fix tissue for direct examination depends on the assay to be performed. In general, in situ hybridization and histology samples are performed in fixed tissues, whereas nucleic acid proliferation techniques (e.g., sequencing) and protein quantification techniques (e.g., ELISA) are performed utilizing extracted biomolecules.

In several embodiments, cells utilized to examine biomolecules are neoplastic cells of a colorectal cancer of an individual, which can be extracted in a biopsy. In some embodiments, a solid tumor biopsy is utilized, such as (for example) a primary, nodal, and/or distal tumor. In some embodiments, a liquid biopsy is utilized to extract ctDNA or CTCs. Sources of liquid biopsies may include blood, plasma, lymph, or any appropriate bodily fluid. The precise source to extract and/or examine biomolecules can depend on the assay to be performed, the availability of a biopsy, and preference of the practitioner.

A number of assays are known to determine genetic aberrations in a biological samples, including (but not limited to) nucleic acid hybridization techniques, nucleic acid proliferation techniques, and nucleic acid sequencing. A number of hybridization techniques can be used, including (but not limited to) ISH, microarrays (e.g., Affymetrix, Santa Clara, Calif.), and NanoString nCounter (Seattle, Wash.). Likewise, a number of nucleic acid proliferation techniques can be used, including (but not limited to) PCR and RT-PCR. In addition, a number of sequencing techniques can be used, including (but not limited to) genome sequencing, exome sequencing, targeted gene sequencing, Sanger sequencing, and RNA-seq of tumor tissue. In several embodiments, the genetic aberrations to be detected are those that can exist within particular combinations of genes that indicate metastatic potential.

As understood in the art, only a portion of a genomic locus or gene may need to be detected in order to have a positive detection. In many hybridization techniques, detection probes are typically between ten and fifty bases, however, the precise length will depend on assay conditions and preferences of the assay developer. In many amplification techniques, amplicons are often between fifty and one-thousand bases, which will also depend on assay conditions and preferences of the assay developer. In many sequencing techniques, genomic loci and transcripts are identified with sequence reads between ten and several hundred bases, which again will depend on assay conditions and preferences of the assay developer. In several embodiments, when a particular genetic aberration is to be detected, only a portion of a genomic locus encompassing the location of the genetic aberration is examined, especially in hybridization and targeted sequencing techniques. In some embodiments, hybridization and targeted sequencing techniques are directed to sequences of a number of genes of interest, such as those that confer an indication of the aggression and metastatic potential of a colorectal cancer.

It should be understood that minor variations in gene sequence and/or assay tools (e.g., hybridization probes, amplification primers) may exist but would be expected to provide similar results in a detection assay. These minor variations are to include (but not limited to) insertions, deletions, single nucleotide polymorphisms, and other variations due to assay design. In some embodiments, detection assays are able to detect genomic loci and transcripts having high homology but not perfect homology (e.g., 70%, 80%, 90%, 95%, or 99% homology). In some embodiments, detection assays are able to detect genomic loci and transcripts having 1 base pair changed, deleted or inserted, 2 base pairs changed, deleted or inserted, 3 base pairs changed, deleted or inserted, 4 base pairs changed, deleted or inserted, 5 base pairs changed, deleted or inserted, or more than 5 base pairs changed, deleted or inserted. As understood in the art, the longer the nucleic acid polymers used for hybridization, less homology is needed for the hybridization to occur.

It should also be understood that several gene transcripts have a number isoforms that are expressed. As understood in the art, many alternative isoforms confer similar indication of molecular classification, and thus metastatic potential. Accordingly, alternative isoforms of gene transcripts are also covered in some embodiments.

In many embodiments, an assay is used to detect genetic aberrations. The results of the assay can be used to determine whether a particular combination of genes harbor genetic aberrations that are indicative of metastatic potential. For example, the NanoString nCounter, which can quantify up to several hundred nucleic acid molecule sequences in one microtube utilizing a set of complement nucleic acids and probes, can be used to determine genetic aberrations of a set of genomic loci and/or gene transcripts. Detection of genetic aberrations in a combination of genes then is used to determine a cancer's metastatic potential, which can be utilized to treat the cancer accordingly.

In some embodiments, when a biopsy is screened for genetic aberrations, the detected aberrations have a known pathogenicity and thus known to confer an oncogenic effect. In some embodiments, a number of genetic aberrations are detected without a known pathogenicity. In some of these embodiments, a pathogenic effect is assumed to confer an oncogenic effect for any genetic aberration within a gene of interest (i.e., a gene known to promote aggressive and/or metastatic cancer). In some embodiments, a computational program is utilized to determine a pathogenic effect, and thus used to determine to likely confer an oncogenic effect. A number of computational programs can be utilized to determine a pathogenic effect, including (but not limited to) VEP (uswest.ensembl.org/Tools/VEP), FATHMM (fathmm.biocompute.org.uk/cancer.html) and CADD (cadd.gs.washington.edu/).

### Kits

In several embodiments, kits are utilized for monitoring individuals for colorectal cancer risk, wherein the kits can be used to detect genetic aberrations in biomarkers as described herein. For example, the kits can be used to detect any one or more of the gene biomarkers described herein, which can be used to determine aggressiveness and metastatic potential. The kit may include one or more agents for determining genetic aberrations, a container for holding a biological sample (e.g., tumor or liquid biopsy) obtained from a subject; and printed instructions for reacting agents with the biological sample to detect the presence or amount of one or more genetic aberrations within biomarker genes derived from the sample. The agents may be packaged in separate containers. The kit may further comprise one or more control reference samples and reagents for performing a biochemical assay, enzymatic assay, immunoassay, hybridization assay, or sequencing assay.

A nucleic acid detection kit, in accordance with various embodiments, includes a set of hybridization-capable complement sequences and/or amplification primers specific for a set of genomic loci and/or expressed transcripts. In some instances, a kit will include further reagents sufficient to facilitate detection and/or quantitation of a set of genomic loci and/or expressed transcripts. In some instances, a kit will be able to detect and/or quantify for at least 5, 10, 15, 20, 25, 30, 40 or 50 loci and/or genes. In some instances, a kit will be able to detect and/or quantify thousands or more genes via a sequencing technique.

In a number of embodiments, a set of hybridization-capable complement sequences are immobilized on an array, such as those designed by Affymetrix or IIlumina. In many embodiments, a set of hybridization-capable complement sequences are linked to a “bar code” to promote detection of hybridized species and provided such that hybridization can be performed in solution, such as those designed by NanoString. In several embodiments, a set of primers (and, in some cases probes) to promote amplification and detection of amplified species are provided such that a PCR can be performed in solution, such as those designed by Applied Biosystems of ThermoScientific (Foster City, Calif.).

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of detecting aberrations from tumor and/or liquid biopsies.

### Applications and Treatments for Colorectal Cancer

Various embodiments are directed to colorectal cancer diagnostics and treatments based on molecular identification and/or characterization of the cancer. As described herein, a screening procedure can utilize a liquid biopsy to identify a colorectal cancer in a patient. In addition, classification of a colorectal cancer by a combination of genes harboring genetic aberrations can be used to determine the aggressiveness metastatic potential of the cancer. Based on the molecular identification and characterization, further diagnostics and or treatments may be administered to a colorectal cancer patient.

### Screening

A number of embodiments are directed towards screening and diagnosing individuals on the basis of their genetic indicators within a liquid biopsy (e.g., blood, plasma, or lymph). In some embodiments, ctDNA and/or CRC cells are extracted from a liquid biopsy and further analyzed.

In a number of embodiments, screening diagnostics can be performed as follows:
a) obtain liquid biopsy of the individual to be screened
b) determine the presence of ctDNA and/or CRC cells
c) perform further diagnostics on individual if ctDNA and/or CRC cells present
d) diagnose the individual based on the presence of and molecular profile of ctDNA and/or CRC cells and any further diagnostics performed.

Screening procedures, in accordance with various embodiments, can be performed as portrayed and described in herein, such as portrayed in FIG. 2. Accordingly, in several embodiment ctDNA and/or CRC cells are utilized to indicate whether a colorectal cancer is present within the individual, as can be determined by identifying the tissue source of the ctDNA and/or CRC cells such that it can be determined if there is a colorectal cancer present. In addition, in many embodiments, the genetic aberrations within the ctDNA and/or CRC cells are examined to determine whether a colorectal cancer is aggressive and/or metastatic.

In accordance with several embodiments, once an indication of colorectal cancer is present, a number of follow-up diagnostic procedures can be performed. In some embodiments, an indication of a highly aggressive and metastatic cancer would indicate that nodal biopsies and body scans looking for metastasis should be performed. Accordingly, in some embodiments, biopsies are retrieved from lymph nodes throughout the body and/or medical imaging can be performed on potential metastatic sites. Medical imaging includes (but is not limited to) endoscopy, X-ray, magnetic resonance imaging (MRI), computed tomography (CT), ultrasound, and positron emission tomography (PET). Endoscopy includes (but is not limited to) bronchoscopy, colonoscopy, colposcopy, cystoscopy, esophagoscopy, gastroscopy, laparoscopy, neuroendoscopy, proctoscopy, and sigmoidoscopy.

### Clinical Diagnostics

A number of embodiments are directed towards diagnosing individuals based on detecting genetic aberrations in genes from a biopsy. In some embodiments, a biopsy is a liquid biopsy in which ctDNA or CRC cells are examined. In some embodiments, a biopsy is a solid biopsy derived from a primary, metastatic, or nodal tumor in which biomolecules are extracted or directly examined within the sample.

In a number of embodiments, colorectal cancer diagnostics can be performed as follows:
a) classify a colorectal cancer into a stage based on primary tumor, regional lymph nodes, and distal metastasis
b) obtain a liquid or tumor biopsy
c) examine biomolecules for genetic aberrations
d) diagnose the individual based on stage and the presence of genetic aberrations.

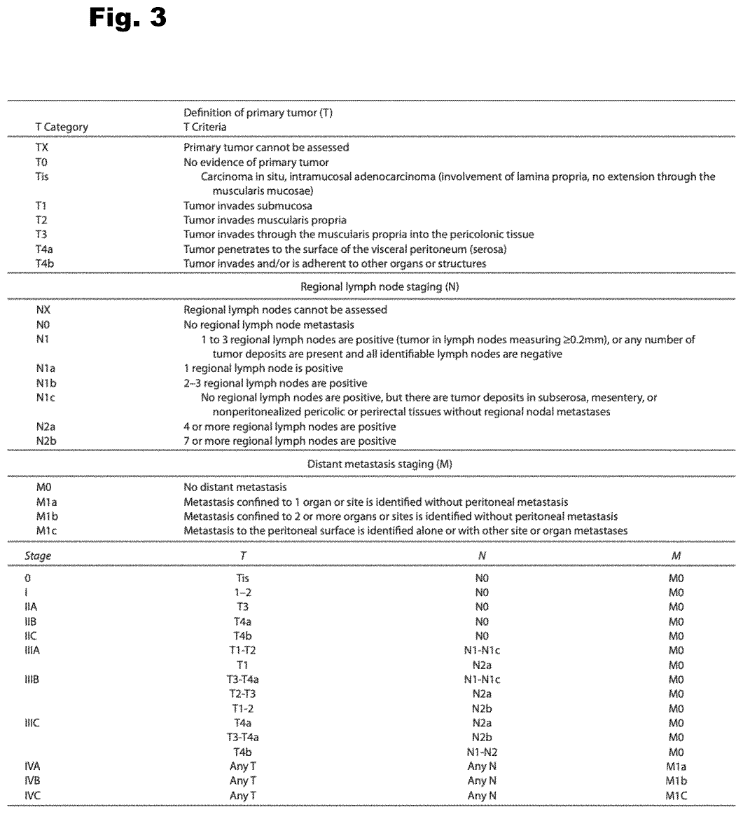

Classification of stage can be performed as would be performed typically within the clinic for colorectal cancer. In general, colorectal cancer can be classified based upon primary tumor invasiveness, number of positive regional lymph nodes, and number of sites of distal metastasis. Provided in FIG. 3 is a table that describes one example of how to classify colorectal cancer (see J. D. Vogel, Dis. Colon Rectum 60, 999-1017 (2017), the disclosure of which is herein incorporated by reference. Any appropriate system to classify a colorectal cancer into a stage can be utilized, in accordance with various embodiments of the invention.

Determination of genetic aberrations, in accordance with various embodiments, can be performed in any appropriate method, including (but not limited to) as portrayed and described in herein, such as portrayed in FIG. 1. Accordingly, a number of gene combinations indicate an aggressive and metastatic phenotype. These gene combinations indicating an aggressive phenotype include the following:
PTPRT+[APC or KRAS or TP53 or SMAD4]
PTPRT+[APC and KRAS]
PRPRT+[APC and TP53]
PTPRT+[TP53 and KRAS]
PTPRT+[TP53 and SMAD4]
PTPRT+[TP53 and KRAS and SMAD4]
AMER1+[APC or KRAS or TP53]
AMER1+[APC and KRAS]
AMER1+[APC and TP53]
TCF7L2+[APC or TP53]
TCF7L2+[APC and TP53]

Based on colorectal cancer stag and the aggressive and metastatic phenotype that is detected, a number of measures can be taken, as discussed within the “Methods of Treatments” section herein. Generally, when an aggressive and metastatic phenotype is detected, a more aggressive treatment approach may be desired as dependent on the stage classification.

### Methods of Treatments

Several embodiments are directed to the use of medical procedures and medications to treat a colorectal cancer based on classification of the cancer. Generally, a diagnosis is performed to indicate the stage of colorectal cancer and/or aggressiveness as determined by genetic aberrations. Based on diagnosis, surgical procedure and course of treatment can be administered.

In accordance with standard procedures, when a colorectal cancer has a Stage 0 classification, a local excision and/or polypectomy is performed. In a number of embodiments, when a colorectal cancer has a Stage 0 classification and further indicates an aggressive phenotype, prolonged monitoring is performed after local excision and/or polypectomy. In some embodiments, when a colorectal cancer has a Stage 0 classification and further indicates an aggressive phenotype, a low dose chemotherapeutic agent is administered, which may help prevent tumor reoccurrence and/or mitigate metastatic spread.

In accordance with standard procedures, when a colorectal cancer has a Stage I classification, a wide surgical resection and anastomosis is performed. In a number of embodiments, when a colorectal cancer has a Stage I classification and further indicates an aggressive phenotype, prolonged monitoring is performed after surgical resection and anastomosis. In some embodiments, when a colorectal cancer has a Stage I classification and further indicates an aggressive phenotype, a chemotherapeutic agent (especially a low dose) is administered, which may help prevent tumor reoccurrence and/or mitigate metastatic spread. In some embodiments, when a colorectal cancer has a Stage I classification and further indicates an aggressive phenotype, a targeted agent is administered, which may help to directly inhibit the aggressive phenotype.

In accordance with standard procedures, when a colorectal cancer has a Stage II classification, a wide surgical resection and anastomosis is performed and adjuvant chemotherapy is considered. When high risk factors are present, such as poorly differentiated histology, lymphatic or vascular invasion, bowel obstruction, perineural invasion, localized perforation, or positive margins, then adjuvant therapy is more heavily considered, but not necessarily recommended. In a number of embodiments, when a colorectal cancer has a Stage II classification and further indicates an aggressive phenotype, adjuvant chemotherapy is administered and in some embodiments, adjuvant chemotherapy is administered for extended periods of 3 to 6 months. In some embodiments, when a colorectal cancer has a Stage II classification and further indicates an aggressive phenotype, a targeted therapy is administered, which may help to directly inhibit the aggressive phenotype.

In accordance with standard procedures, when a colorectal cancer has a Stage III classification, a wide surgical resection and anastomosis and adjuvant chemotherapy is administered. When high risk factors are present, such as multiple positive regional nodes, then more aggressive and longer adjuvant therapy is administered. In a number of embodiments, when a colorectal cancer has a Stage III classification and further indicates an aggressive phenotype, prolonged adjuvant chemotherapy is administered for extended periods of 3 to 6 months. In some embodiments, when a colorectal cancer has a Stage III classification and further indicates an aggressive phenotype, adjuvant chemotherapy that is typically reserved for metastatic colorectal cancer is administered. In some embodiments, when a colorectal cancer has a Stage III classification and further indicates an aggressive phenotype, a targeted therapy is administered, which may help to directly inhibit the aggressive phenotype.

In accordance with standard procedures, when a colorectal cancer has a Stage IV (metastatic) classification, a wide surgical resection and anastomosis (if resectable) and adjuvant chemotherapy is administered for extended periods of 12 or more months. In a number of embodiments, when a colorectal cancer has a Stage IV classification and further indicates an aggressive phenotype, adjuvant chemotherapy and a targeted therapy is administered, which may help to directly inhibit the aggressive phenotype.

A number of therapeutic agents are available to treat neoplasms and cancers, such radiotherapy, chemotherapy, immunotherapy, and targeted therapy. Chemotherapeutics for non-metastatic colorectal cancer include (but are not limited to) fluorouracil (or 5-fluorouracil or 5-FU), capecitabine, leucovorin, folinic acid, and oxaliplatin. Chemotherapeutics for metastatic colorectal cancer include (but are not limited to) 5-FU, leucovorin, irinotecan, bevacizumab, ziv-aflibercept, cetuximab, panitumumab, nivolumab, pembrolizumab, vemurafenib, ramucirumab, regorafenib, and trifluridine with tipiracil.

For targeted therapy, when PTPRT is indicated as having genetic aberrations, drugs that specifically target the STAT3 pathway can be utilized, which include (but are not limited to) bruceantinol, curcumin, ruxolitinib, golotimod, and AZD9150. When AMER1 or TCF7L2 is indicated as having genetic aberrations, drugs that specifically target the Wnt pathway can be utilized, which include (but are not limited to) SM08502, Lgk974, ETC-159, Wnt-059, and IWP-2. When KRAS is indicated as having genetic aberrations, drugs that specifically target the KRAS pathway can be utilized, which include (but are not limited to) AMG 510 and MRTX849.

Accordingly, an individual may be treated, in accordance with various embodiments, by a single medication or a combination of medications described herein. Common treatment combinations include (but are not limited to) is leucovorin, 5-FU, and irinotecan (FOLFIRI); folinic acid, 5-FU, and oxaliplatin (FOLFOX); and capecitabine and oxaliplatin (CAPEOX).

Dosing and therapeutic regimes can be administered appropriate to the neoplasm to be treated, as understood by those skilled in the art. For example, 5-FU can be administered intravenously at dosages between 25 mg/m² and 1000 mg/m².

In some embodiments, medications are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size and/or risk of relapse.

A therapeutically effective amount can be an amount sufficient to prevent reduce, ameliorate or eliminate the symptoms of colorectal cancer. In some embodiments, a therapeutically effective amount is an amount sufficient to reduce the growth and/or metastasis of a colorectal cancer.

### EXEMPLARY EMBODIMENTS

The embodiments of the invention will be better understood with the several examples provided within. Many exemplary results of processes that identify combinatorial molecular indicators of colorectal cancer are described. Validation results are also provided.

### Example 1

### Quantitative Evidence for Early Metastatic Seeding in Colorectal Cancer

Colorectal cancer (CRC) is the third most commonly diagnosed cancer and leading cause of cancer death, as well as an excellent model for studying tumor progression given that the initiating driver alterations are well characterized. The site and resectability of CRC metastases dictate treatment options and prognosis with liver being the most common metastatic site with one third of metastatic CRC (mCRC) patients exhibiting liver-exclusive metastasis. In contrast, brain metastasis is a rare (<4% of mCRCs), but devastating diagnosis with limited therapeutic options and median survival of 3 to 6 months. In CRC, metastasis is assumed to be seeded by genetically advanced cancer cells that have evolved through a series of sequential clonal expansions. However, CRC progression is not necessarily linear. Rather, within this example a Big Bang model of tumor evolution is described, whereby after transformation some CRCs grow as a single expansion populated by heterogeneous and effectively equally fit subclones, and where most detectable intra-tumor heterogeneity arises early. These data suggest that some CRCs are “born to be bad,” wherein invasive and even metastatic potential is specified early. Effectively neutral evolution has since been reported in other primary tumors, but the ‘mode’ of evolution (effective neutrality versus subclonal selection) has not been evaluated in paired primary tumors and metastases.

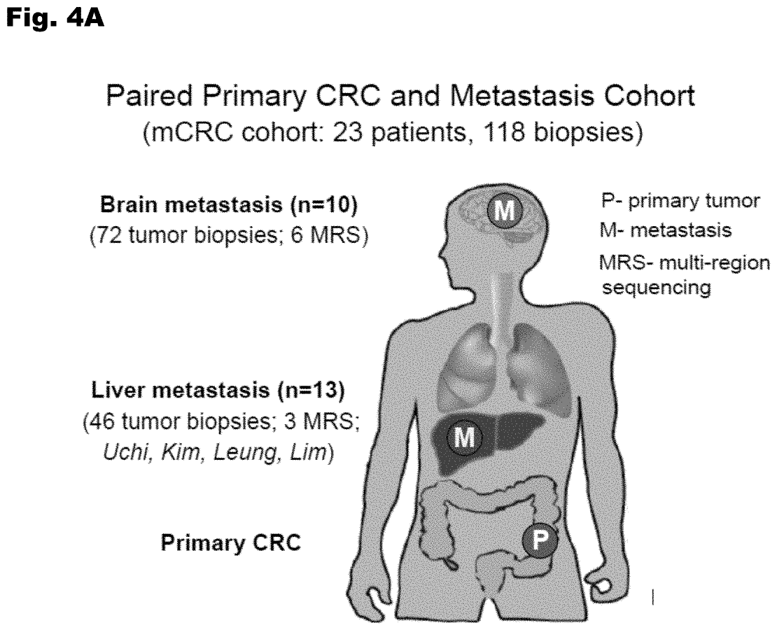

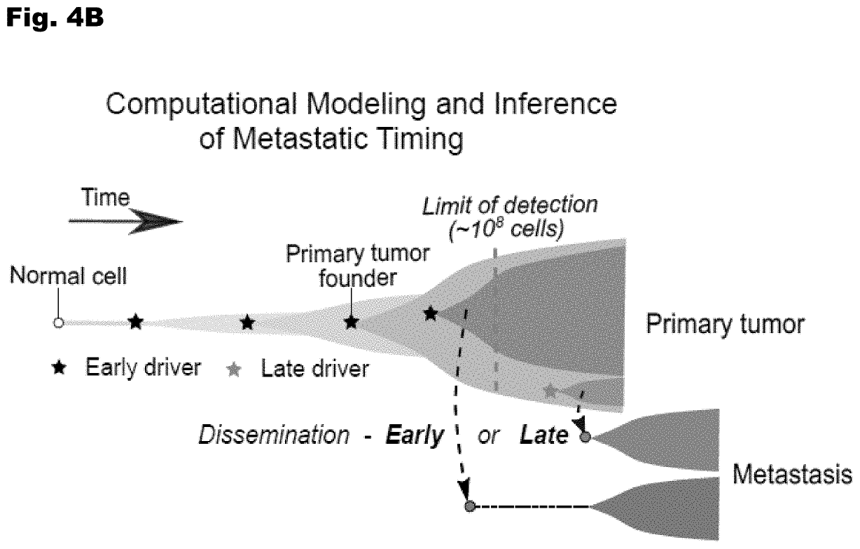

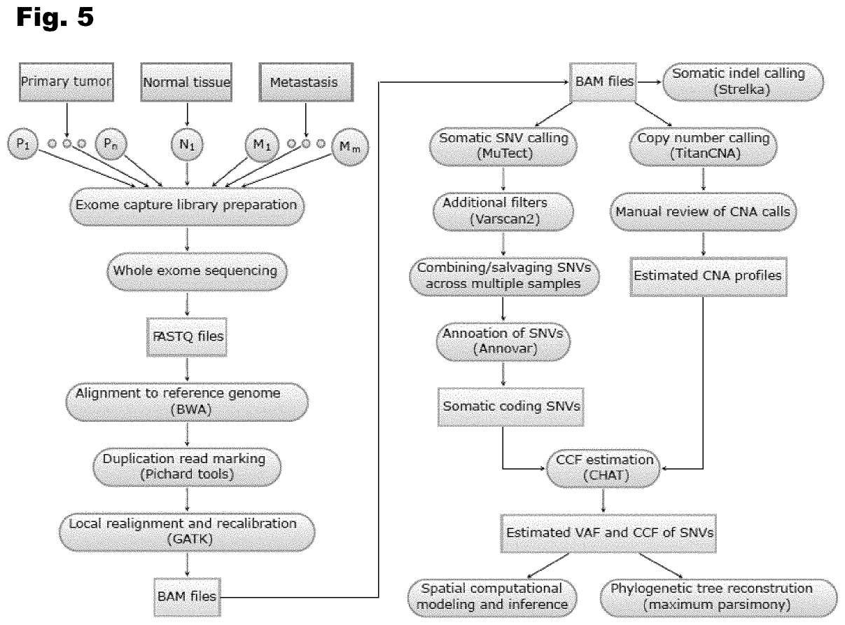

Although the metastatic process is largely occult, spatio-temporal patterns of genomic variation in paired primary tumors and metastases embed their evolutionary histories. In this example, exome sequencing data from 118 biopsies from 23 mCRC patients with paired distant metastases to the liver or brain to delineate the timing and routes of metastasis and to define metastasis competent clones were analyze (FIGS. 4A and 4B). The data show low primary tumor-metastasis genomic divergence (PMGD), where genomic drivers were acquired early. Moreover, through simulation studies, it was established that low PMGD in bulk-sample sequencing data is indicative of early dissemination, contrary to current assumptions. Phylogeny reconstruction and analysis of the mutational cancer cell fraction (CCF) revealed the early divergence of metastatic lineages and their monoclonal origin. To overcome the limitations of phylogenetic approaches, which cannot resolve the timing of dissemination, a spatial computational model of tumor progression and Bayesian statistical inference framework to ‘time’ dissemination in a patient-specific fashion was developed. Further, we validated the association between combinations of early driver genes and metastasis in an independent cohort of 2,751 CRCs, demonstrating their utility as biomarkers of aggressive disease.

Furthermore, analysis within a spatial tumor growth model and statistical inference framework indicates that early disseminated cells commonly (81%, 17/21 evaluable patients) seed metastases while the carcinoma is clinically undetectable (typically <0.01 cm³). The association between early drivers and metastasis was validated in an independent cohort of 2,751 CRCs, demonstrating their utility as biomarkers of metastasis. This new conceptual and analytical framework provides quantitative in vivo evidence that systemic metastatic seeding can occur early in CRC and illuminates strategies for patient stratification and therapeutic targeting of the canonical drivers of tumorigenesis for systemic therapy and earlier detection.

### Overview of Clinical Cohorts

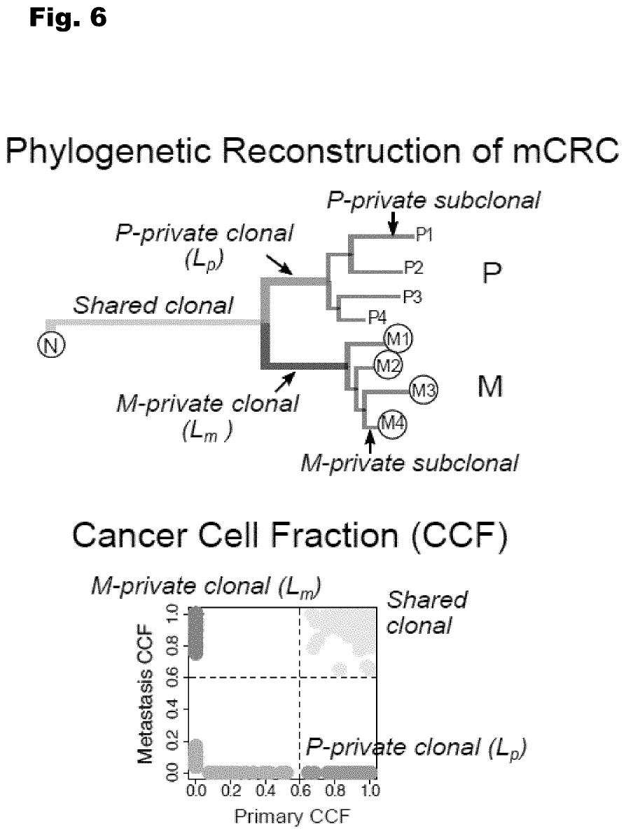

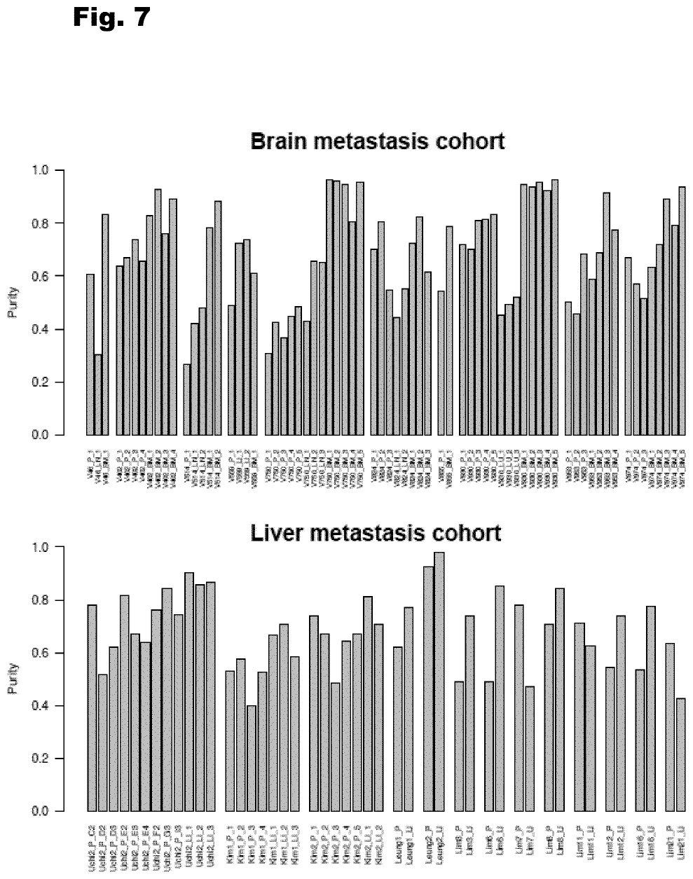

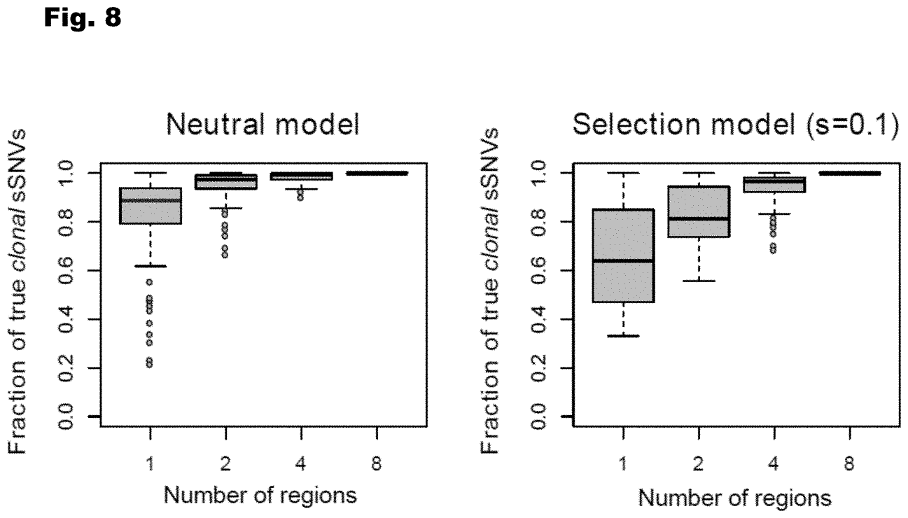

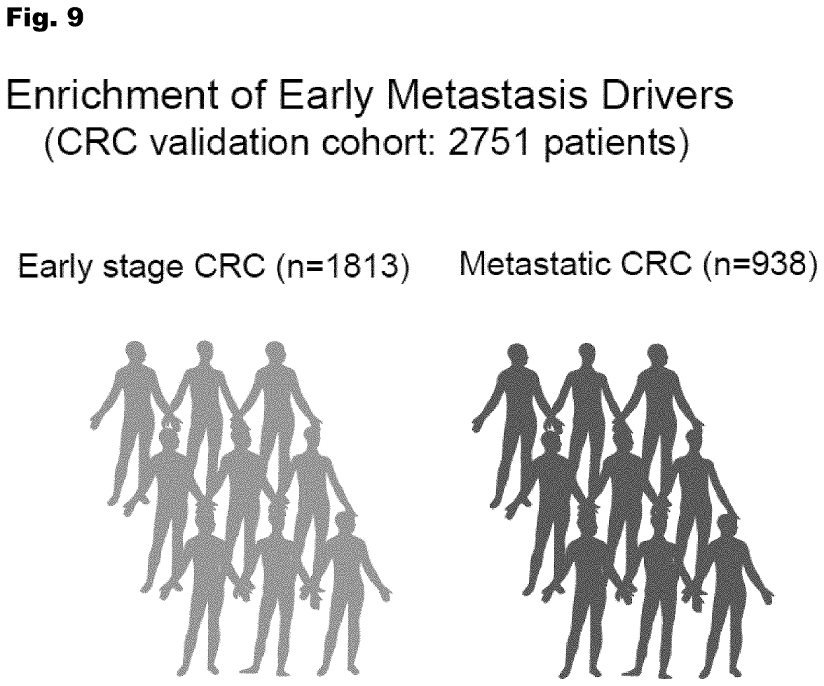

mCRC patients exhibit varied progression paths where liver-exclusive metastasis and brain metastasis represent extreme scenarios with distinct prognoses. It was therefore sought to characterize the genomic landscape, routes and timing of metastasis in mCRC by analyzing exome sequencing data from 118 biopsies from 23 patients with paired distant metastases to the liver or brain (referred to as the mCRC cohort, see FIGS. 4A and 5, and Table 1). To investigate these patterns, sequencing was performed on 72 samples from a unique cohort of 10 mCRC patients with paired brain metastases some of whom had additional metastases to the liver (n=1), lung (n=1) and lymph nodes (n=4). Five patients had brain-exclusive distant metastasis (V402, V514, V855, V953 and V974) estimated to occur in a mere 2-10% of patients with brain metastasis. For six patients, multi-region sequencing (MRS) of the paired primary and metastasis (P/M pairs) was performed (3-5 regions each), enabling the detailed reconstruction of tumor phylogenies (FIG. 6). Additionally, also included were 46 tumor biopsies from 13 mCRC patients with paired liver metastases after excluding cases with low tumor cell purity (<0.4) (FIG. 7) from four published datasets (Uchi, Kim, Leung, and Lim), analyzed using the same unified bioinformatics framework (see Methods section below for details on bioinformatics framework; for more on the published data sets, see R Uchi, et al. PLoS Genet 12, e1005778 (2016); T. M. Kim, et al., Clin Cancer Res 21, 4461-72 (2015); M. L. Leung, et al., Genome Res 27, 1287-1299 (2017); and B. Lim et al., Oncotarget 6, 22179-90 (2015), the disclosures of which are each herein incorporated by reference). No other sites of metastasis were reported for these patients and MRS was available for 3 P/M pairs (n=2-9 regions each). MRS enables more accurate estimation of the cancer cell fraction (CCF) and discrimination between clonal and subclonal mutations relative to single sample sequencing (FIGS. 6 and 8). Additionally, an independent collection of 2,751 CRC patients was leveraged, including 938 with metastatic disease (stage IV) and 1,813 early stage (stage I-III) patients for whom targeted sequencing data from the MSK-Impact and GENIE studies were available in order to evaluate the association between specific combinations of early driver genes (modules) defined in the mCRC cohort and metastatic propensity (FIG. 9; for more on MSK-Impact and GENIE, see R. Yaeger, et al., Cancer Cell 33, 125-136 e3 (2018); and A. P. G. Consortium, Cancer Discov 7, 818-831 (2017); the disclosure of which are each herein incorporated by referenced).

Genomic Heterogeneity in CRCs and Paired Metastases

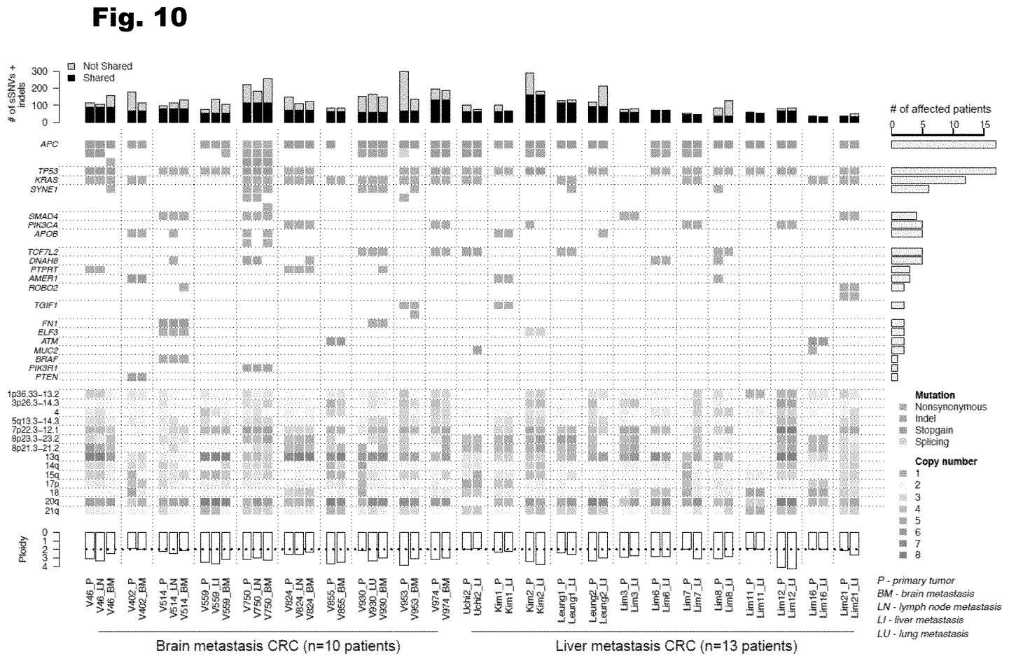

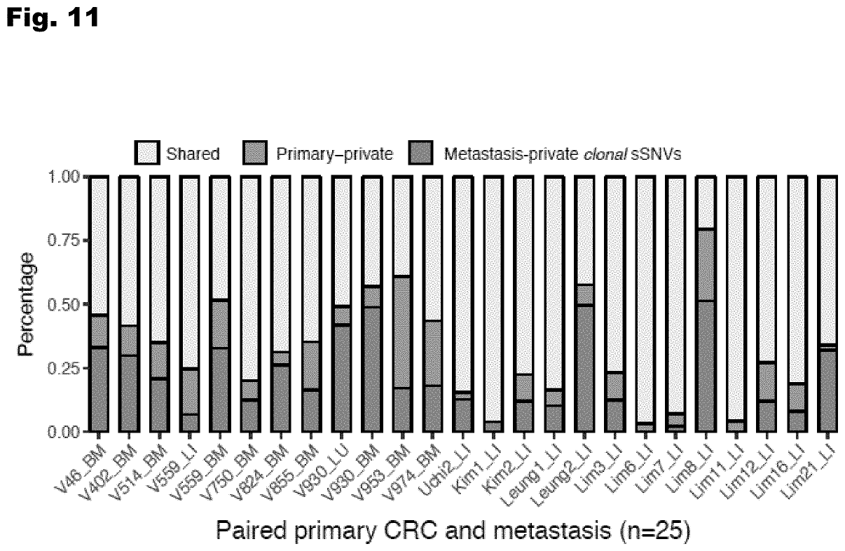

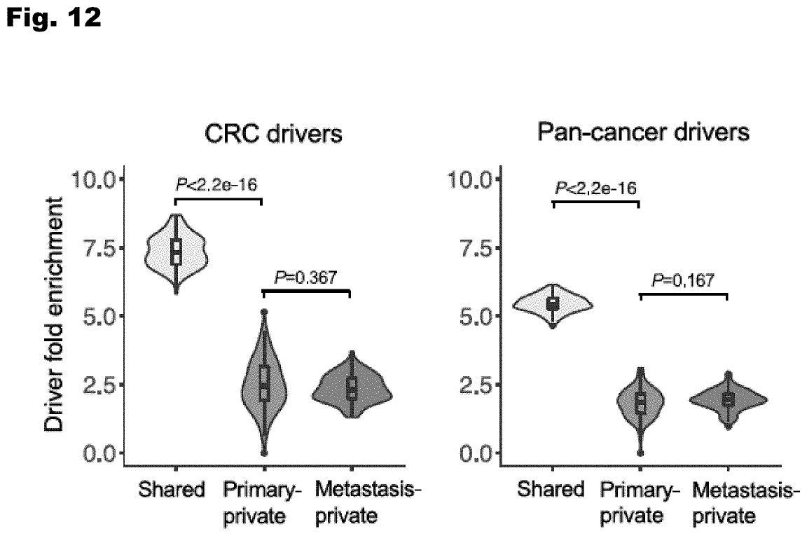

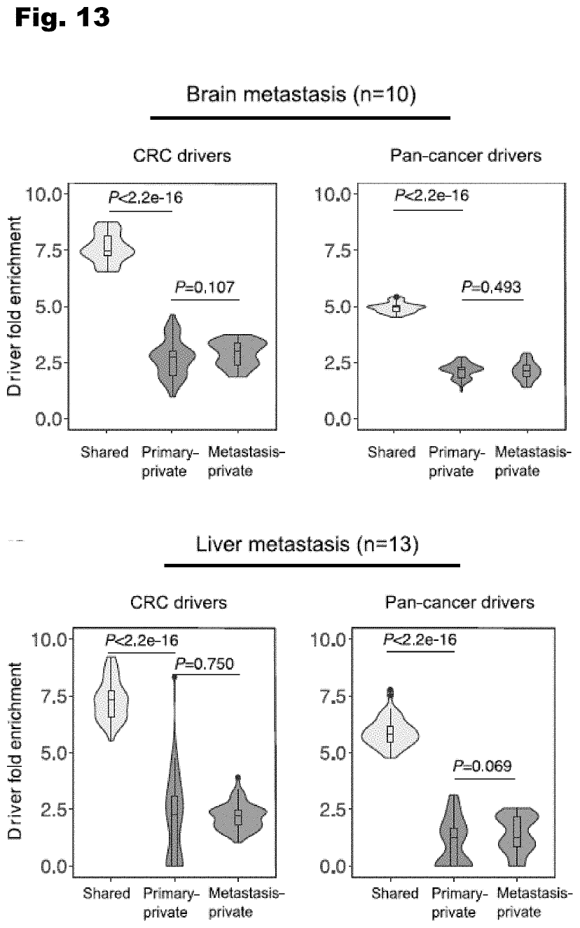

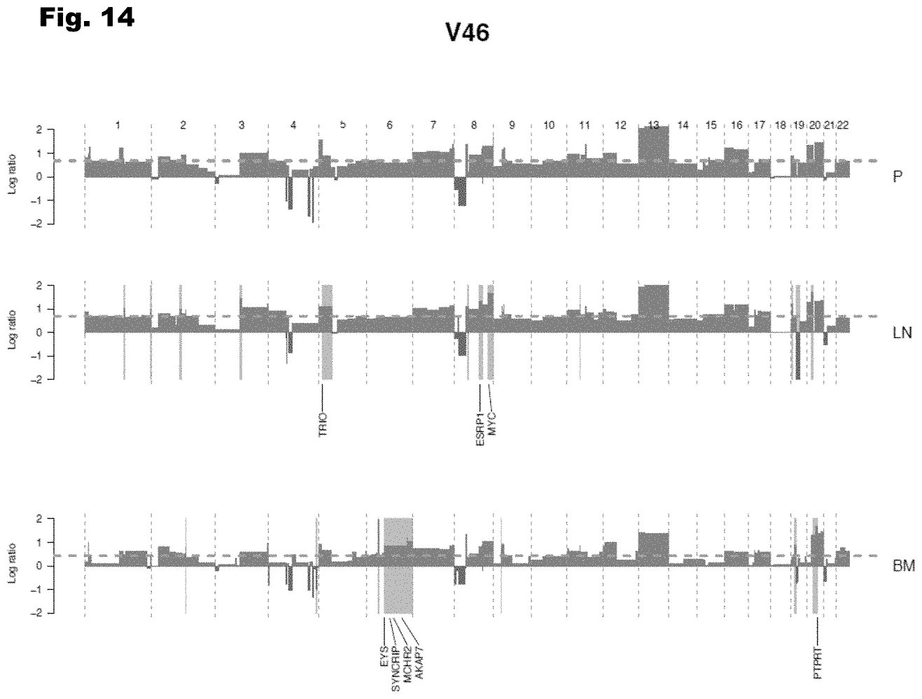

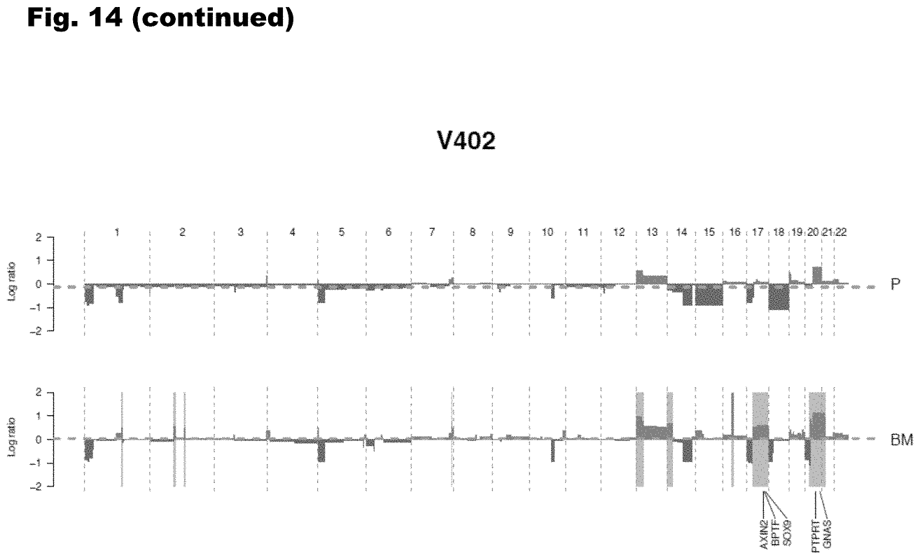

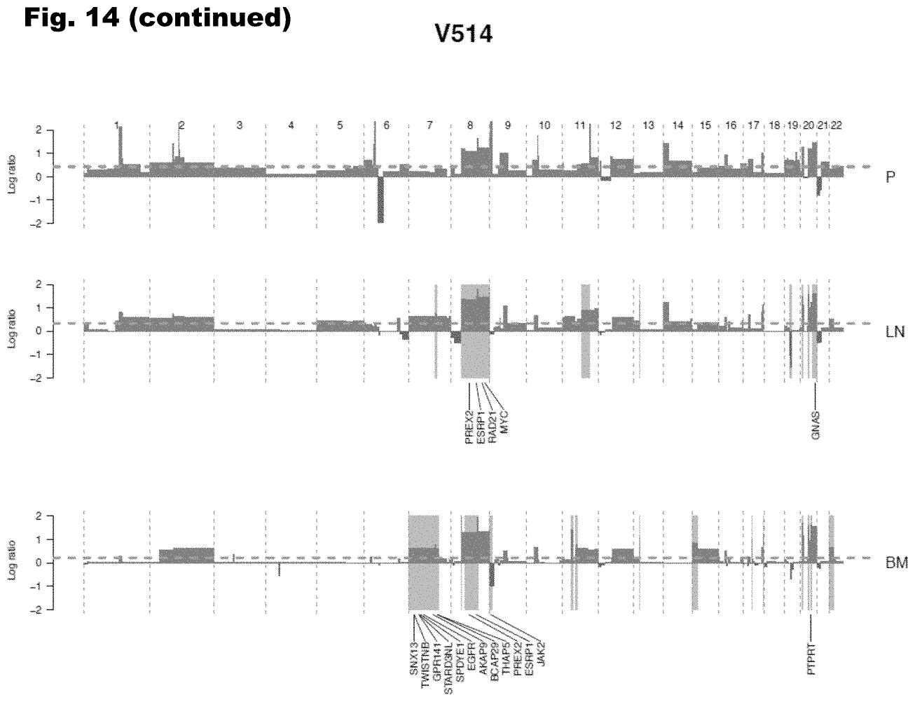

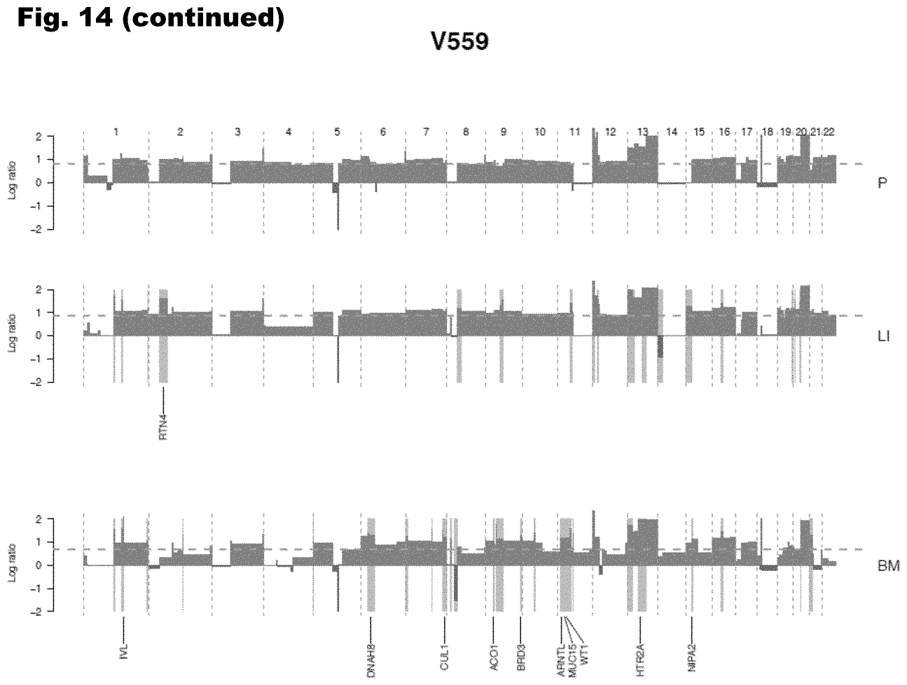

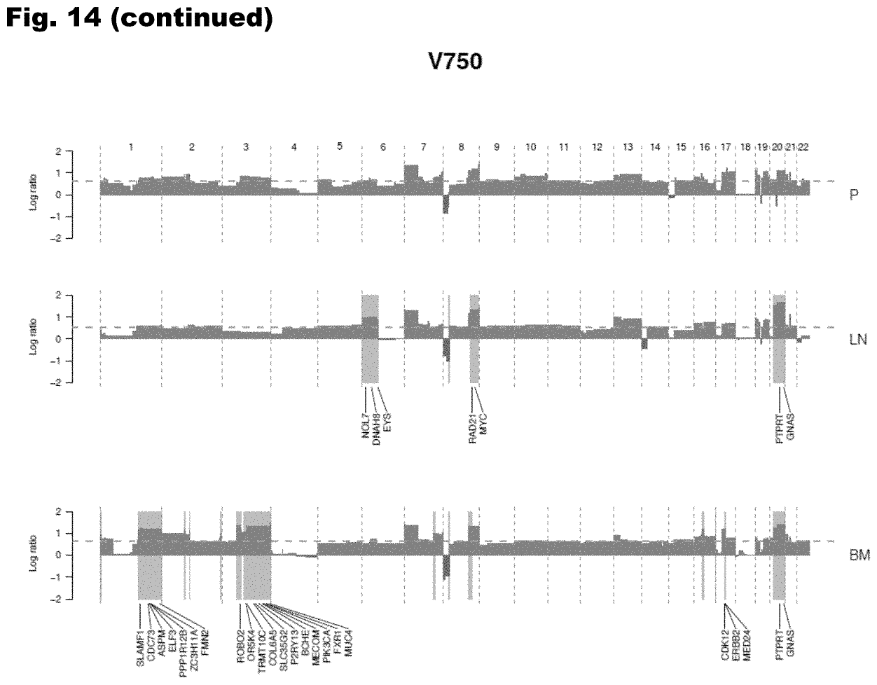

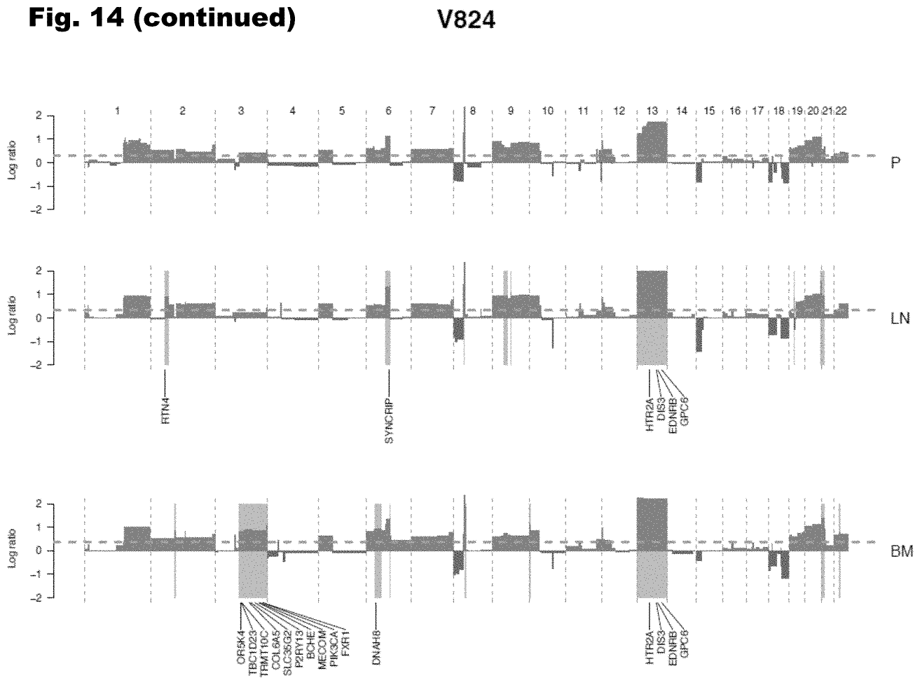

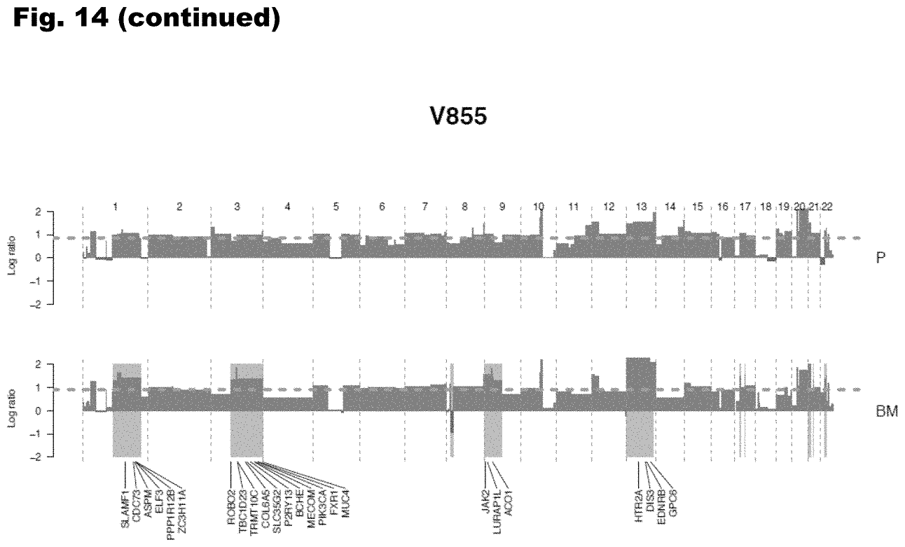

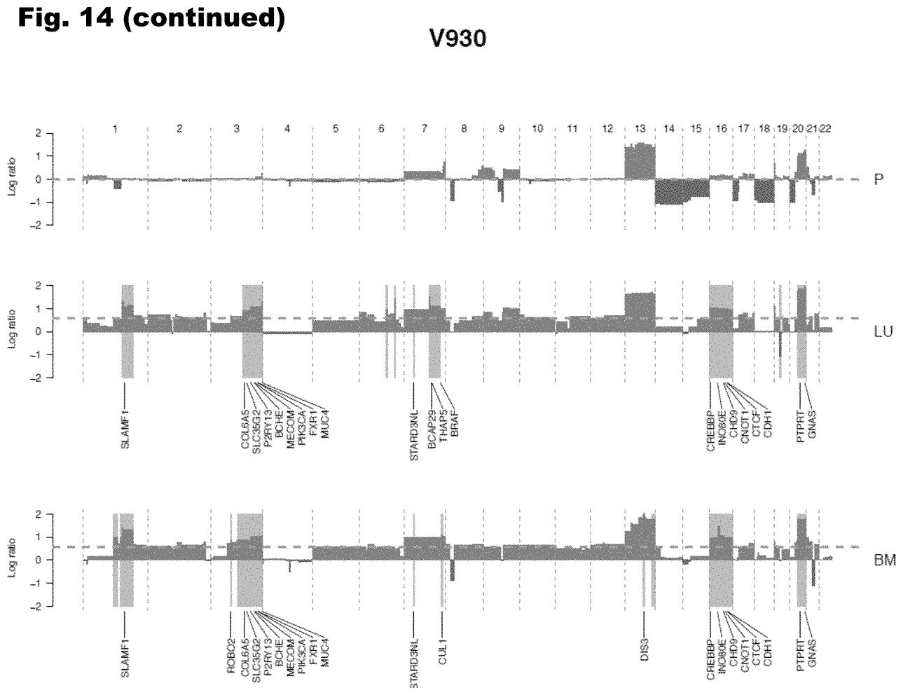

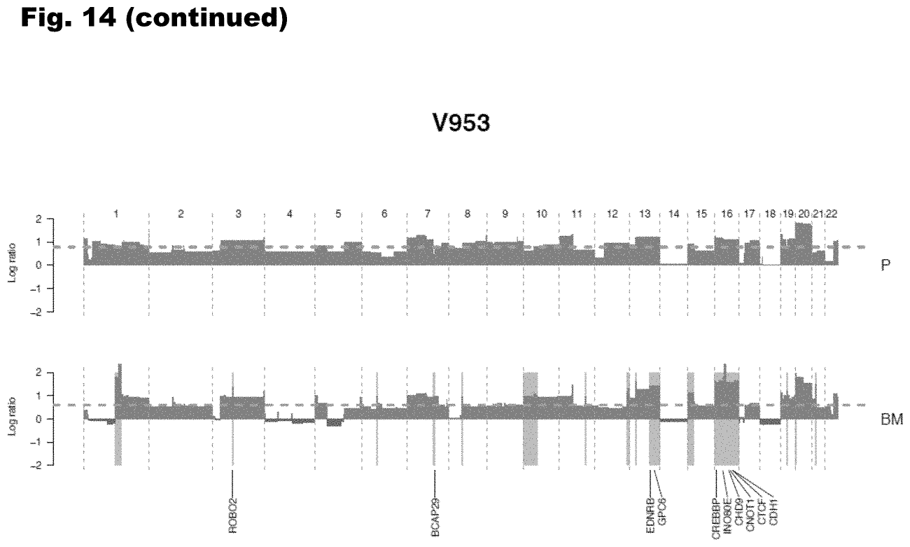

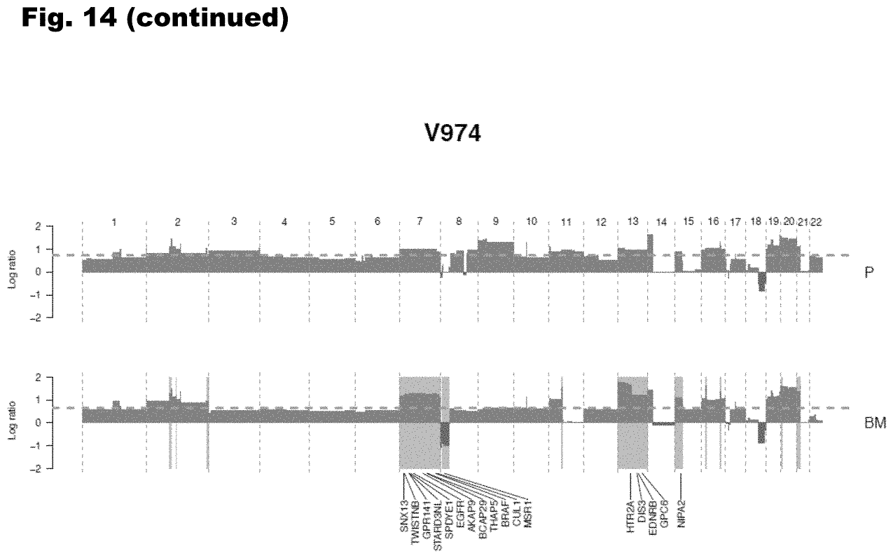

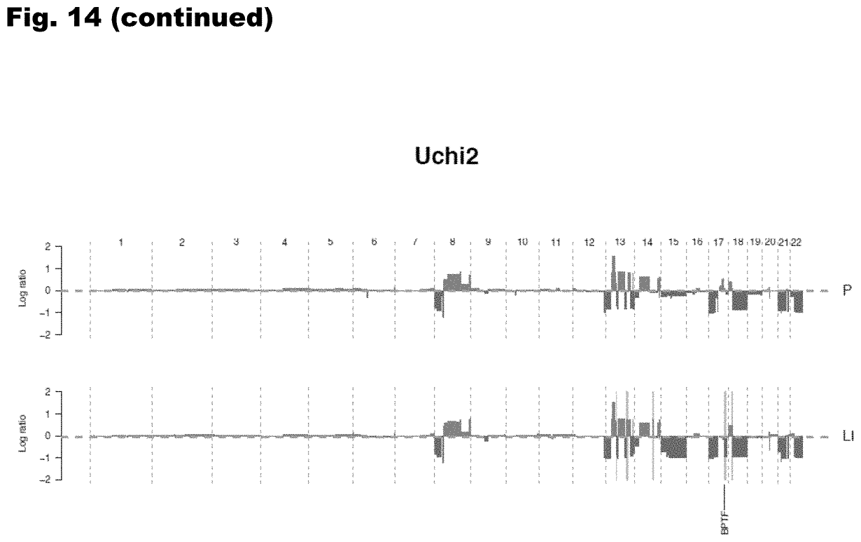

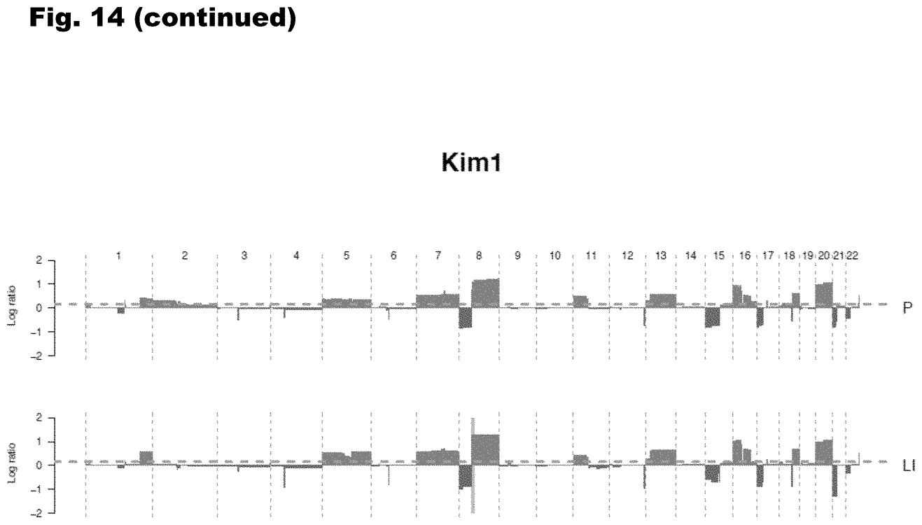

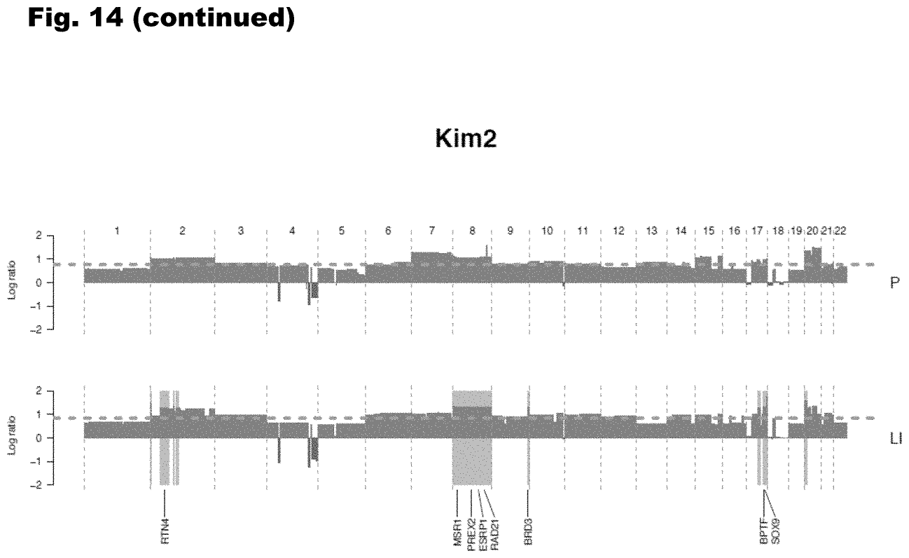

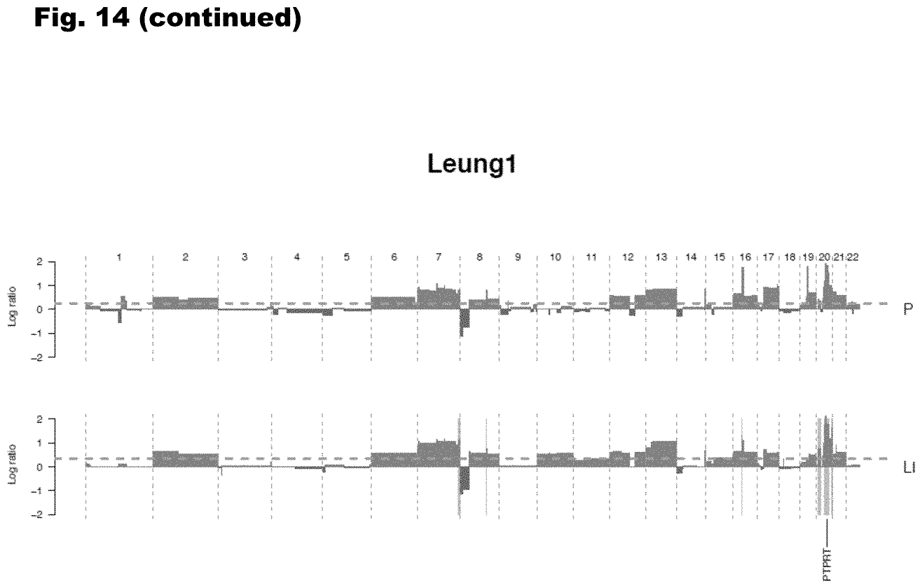

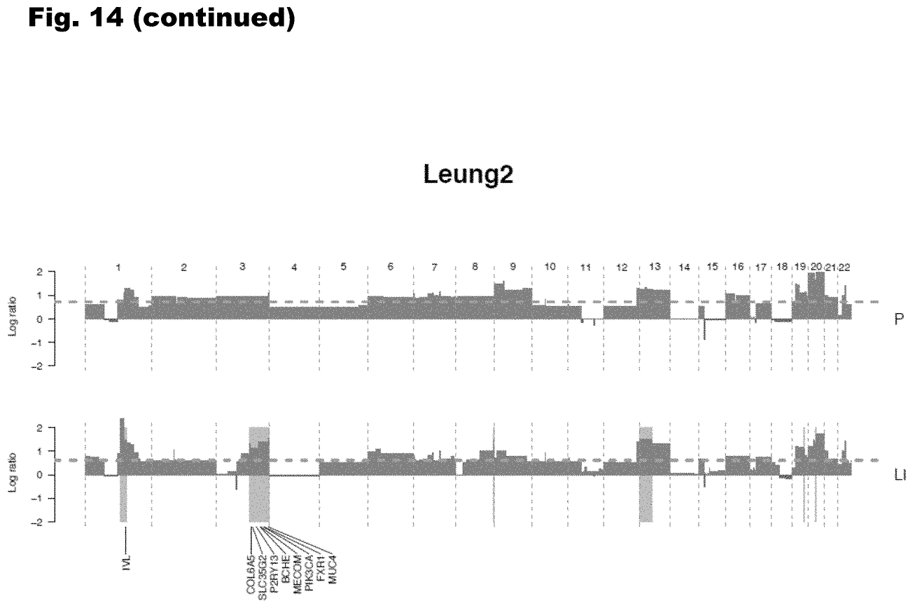

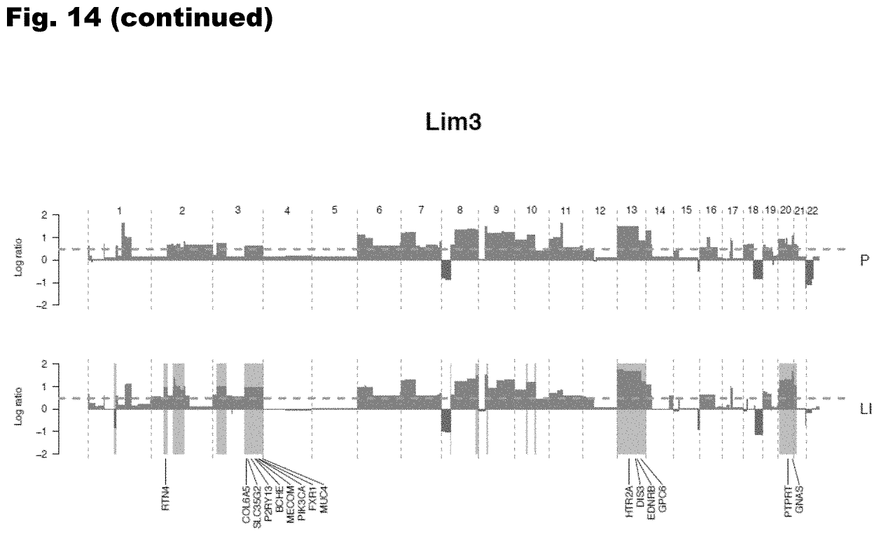

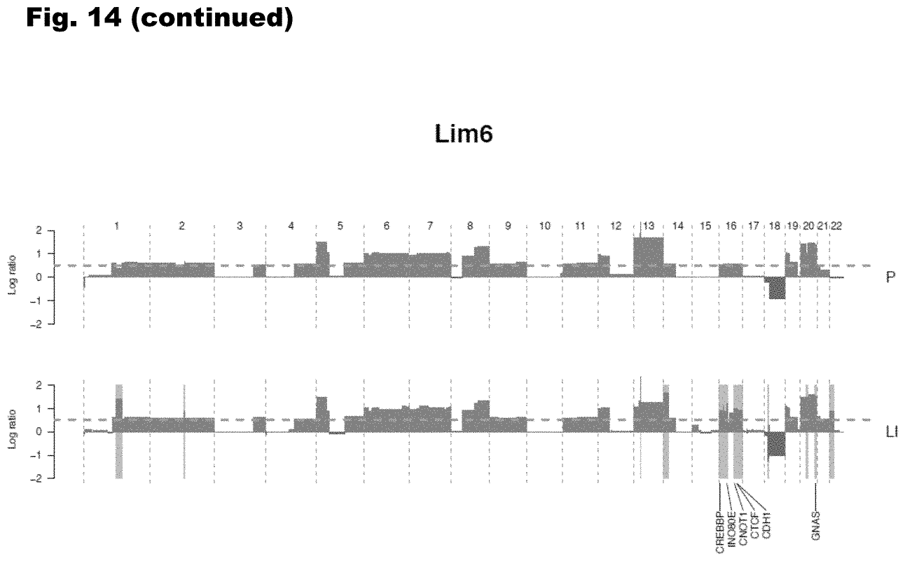

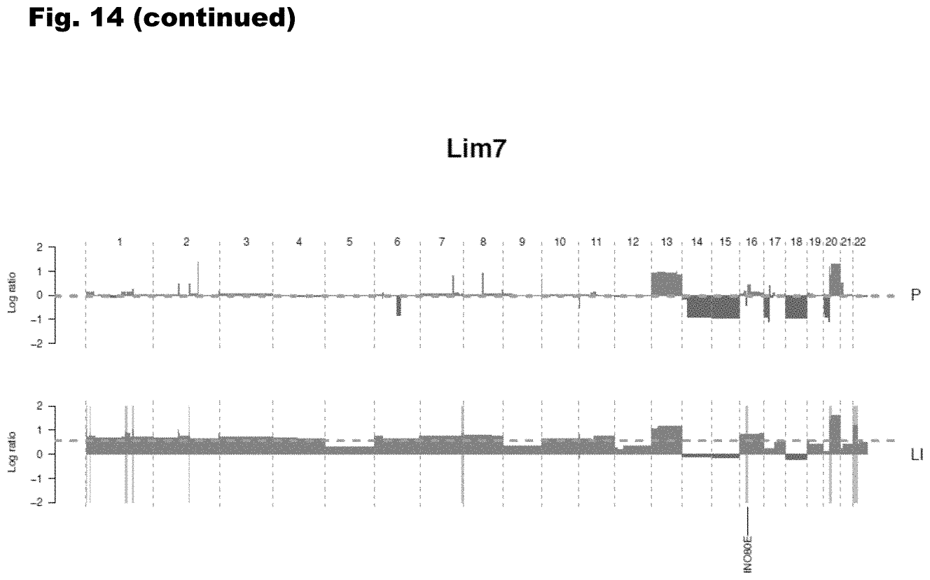

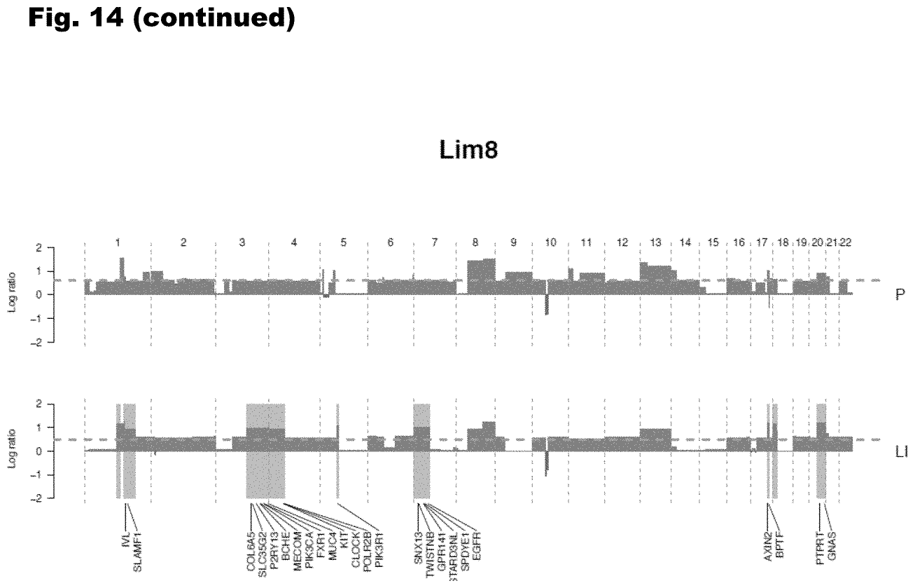

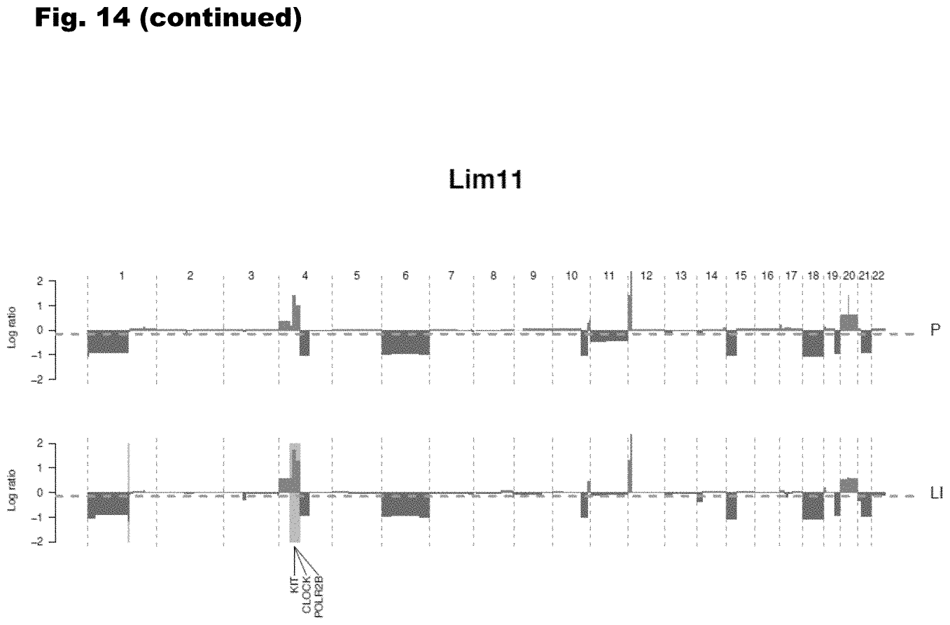

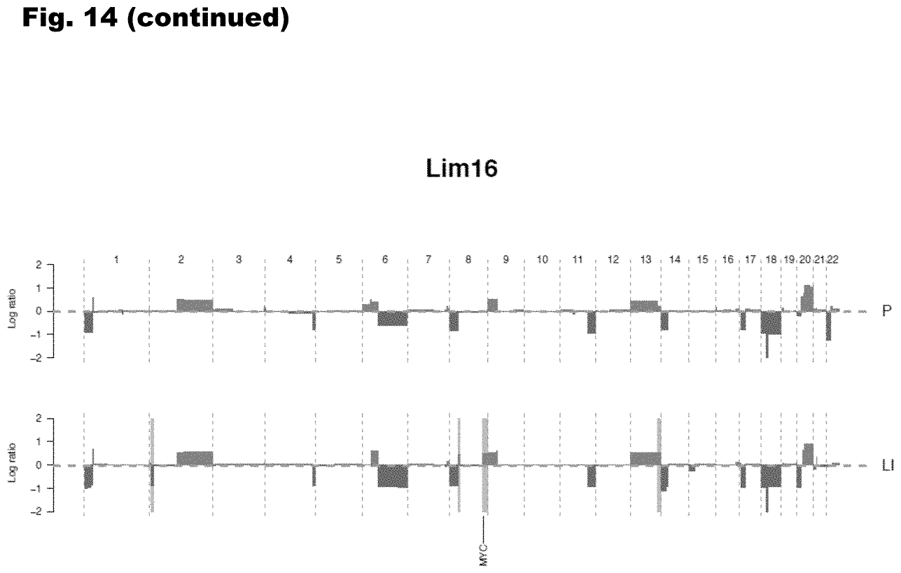

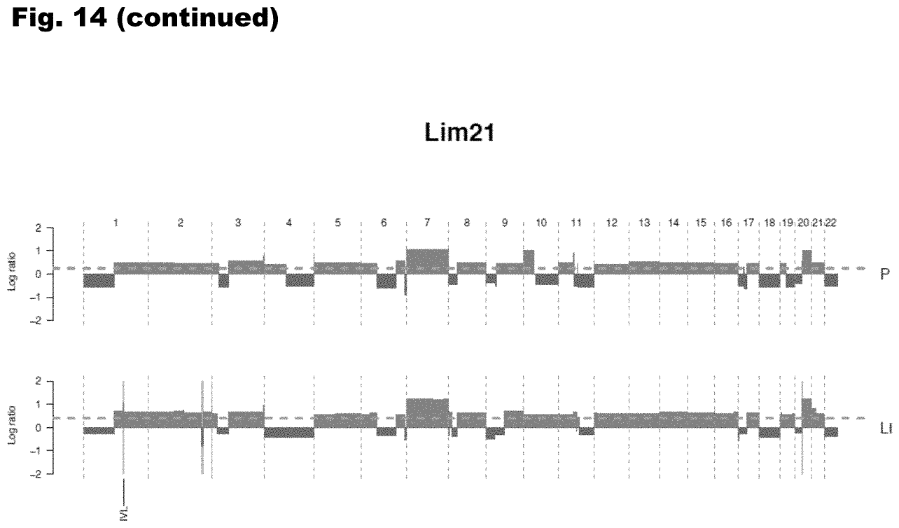

High concordance amongst putative driver genes was observed in the mCRC cohort (FIG. 10). For instance, KRAS, TP53, SMAD4, TCF7L2, FN1, ELF3 and ATM mutations were completely concordant between P/M pairs. On average, 70% of high-frequency somatic single nucleotide variants (sSNVs) with CCF>60% in any primary tumor or metastasis were shared by both lesions (FIG. 11). Amongst genes that were mutated in more than five patients, SYNE1 (4/6 patients) and APOB (3/5 patients) tended to be primary or metastasis private and thus likely arose after transformation. Although metastases usually had more private high-frequency sSNVs than the primary tumor (P=0.020, Wilcoxon Rank-Sum Test, FIG. 11), they were not enriched for CRC drivers (defined based on IntOGen and TCGA) or a published list of pan-cancer drivers (FIG. 12, Table 2, for more on IntOGen and TCGA, see A. Gonzalez-Perez, et al., Nat Methods 10, 1081-2 (2013); and T. C. G. A. Network, Nature 487, 330-7 (2012); the disclosures of which are each herein incorporated by reference). Similar results were obtained when stratifying by brain or liver metastases (FIG. 13). These data reflect limited driver gene heterogeneity between P/M pairs and suggest that few additional private genomic drivers were required for metastasis. Somatic copy number alterations (CNAs) were also generally concordant, with chromosomes 7p22.3-12.1, 13 and 20q11-13 exhibiting recurrent amplification and chromosomes 8p23.3-23.2, 8p21.3-21.2, 18 exhibiting recurrent deletion in P/M pairs (FIGS. 10 and 14). Several putative oncogenes, including PIK3CA, GNAS, SRC, FXR1, MUC4, GPC6, MECOM were recurrently (>4 patients) amplified in metastases relative to paired primary tumors. Intriguingly, HTR2A (5-hydroxytryptamine receptor 2A), which encodes a receptor for the neurotransmitter serotonin that dually functions as a regulatory factor in the gastrointestinal tract, was amplified more frequently in brain (4/10) than liver (1/13) metastases (FIG. 14). These recurrently copy number altered genes may contribute to disease aggressiveness and the propensity to metastasize and represent another means of disrupting a critical pathway. For example, PIK3CA is amplified in some colorectal cancers and harbors activating mutations in others.

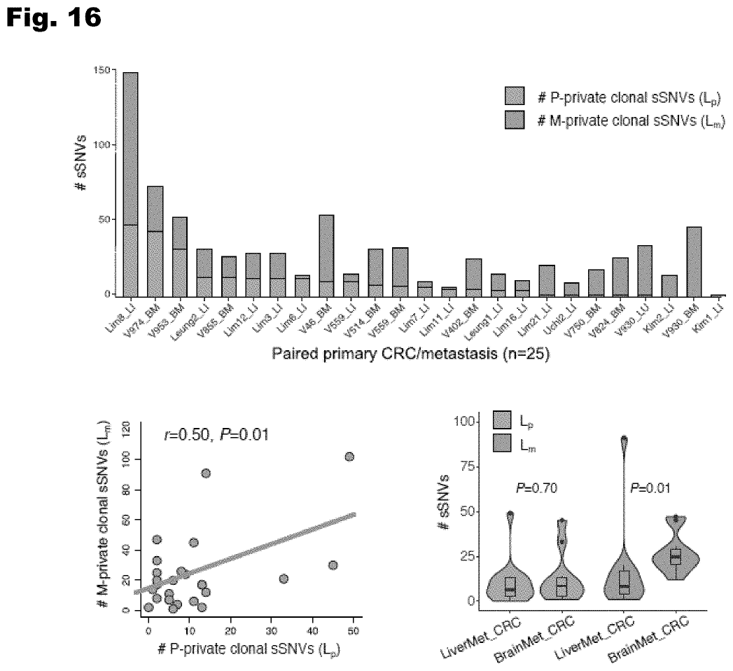

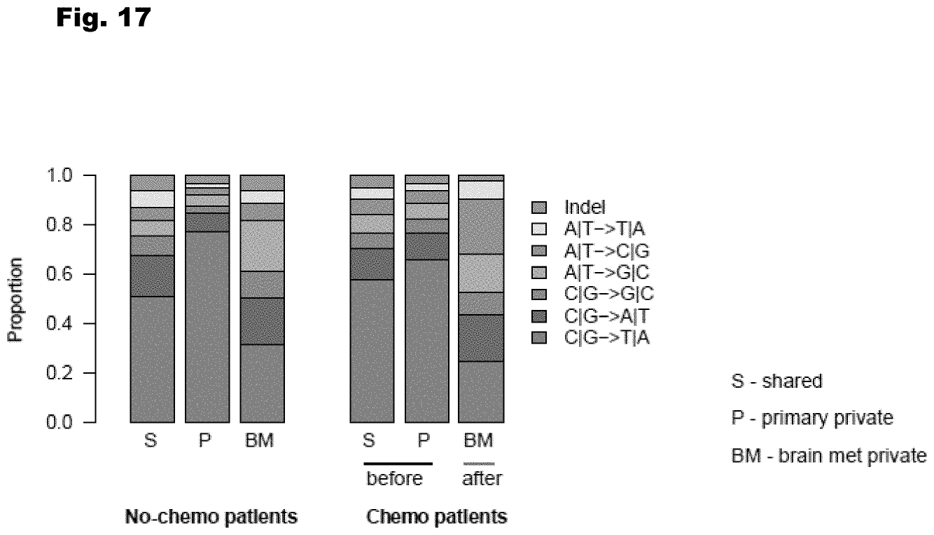

The number of metastasis-private (M-private) clonal sSNVs was defined as Lₘ (merged CCF>60% in the metastasis samples and <1% in the primary tumor samples) and the number of primary tumor-private (P-private) clonal sSNVs as Lₚ (merged CCF>60% in the primary and <1% in the metastasis), where a cutoff of 60% accurately distinguished clonal and subclonal sSNVs (FIGS. 6, 15 and 16). Therefore, a merged CCF value of 60% was used as the cutoff to distinguish clonal and subclonal mutations throughout. Brain metastases exhibited higher Lₘ than liver metastases (median=24.5 vs 9.5, P=0.01, Wilcoxon Rank-Sum Test), whereas no difference was noted for Lₚ (median=8.5 vs. 6.0, P=0.70, Wilcoxon Rank-Sum Test) (FIG. 16), potentially reflecting longer progression times (and more cell divisions). Neither Lₘ (P=0.68, Wilcoxon Rank-Sum Test) nor Lₚ (P=0.95, Wilcoxon Rank-Sum Test) differed significantly in chemo-naïve versus treated cases despite a slight shift in mutational spectra (AIT→CIG) after chemotherapy (FIG. 17).

Gene-ontology analysis showed enrichment for cellular adhesion terms amongst both brain and liver metastasis-private non-silent clonal mutations, but not primary-private clonal or subclonal mutations. Nervous system development and neuronal differentiation terms were enriched amongst brain and liver metastasis-private clonal mutations and primary tumor-private mutations, consistent with hijacking of the enteric nervous system in gastrointestinal malignancies. In contrast, primary tumor-private non-silent clonal mutations were enriched for metabolic processes, DNA repair and damage, suggestive of more general deregulation and resource constraints during tumor expansion.

### Phylogenetic Reconstruction of Metastatic CRC

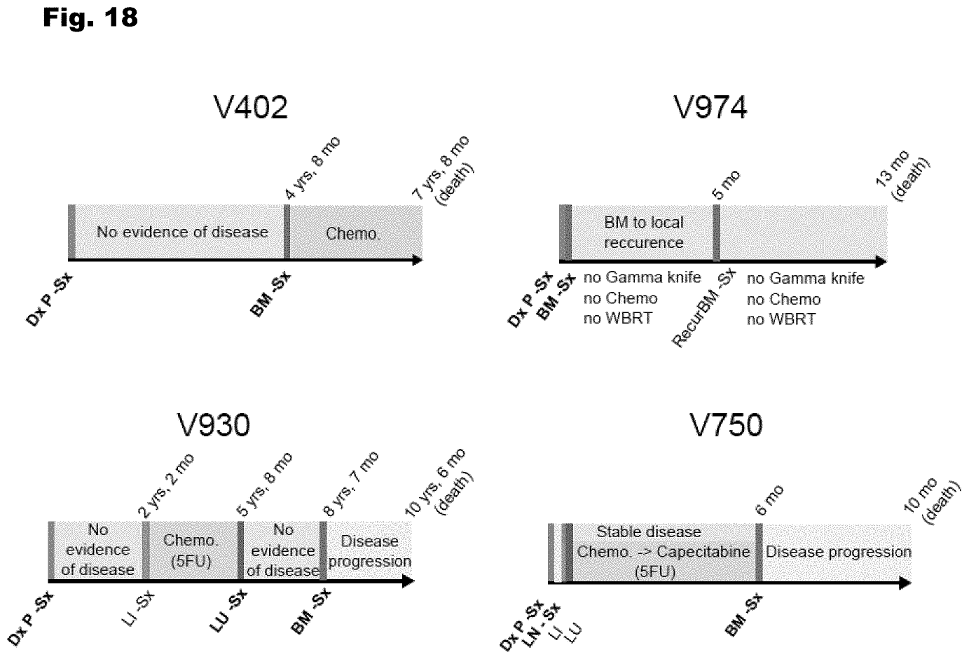

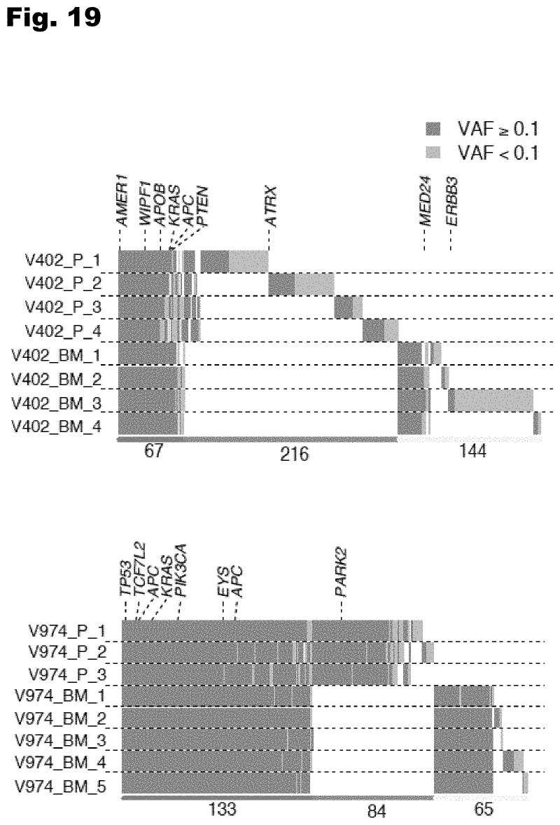

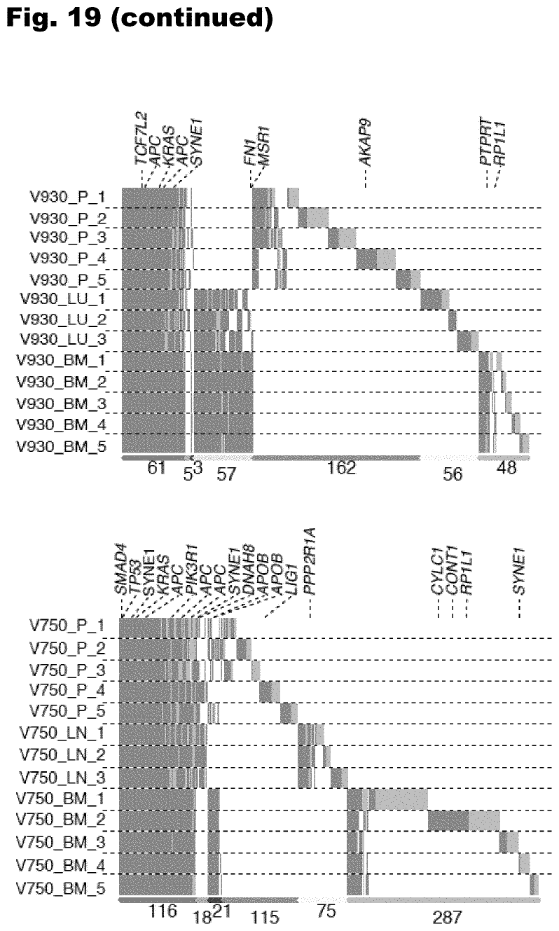

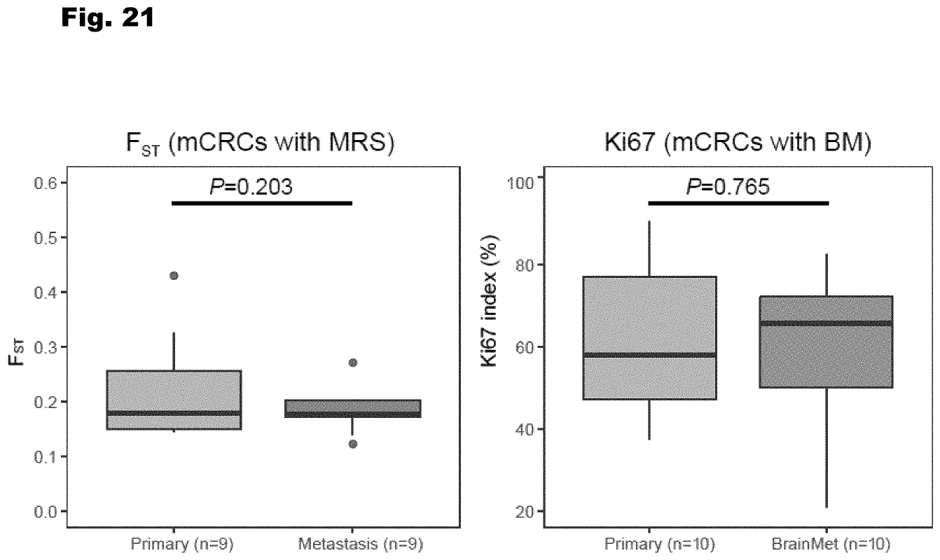

The MRS data revealed extensive intra-tumor heterogeneity (ITH) both within tumors and between P/M pairs (FIGS. 18, 19, and 20) and ample mutations for phylogeny reconstruction. F_{ST} was employed to quantify ITH within tumors (primary tumor or metastasis) in the mCRC cohort based on subclonal sSNVs. Clonal mutations present in all samples do not contribute to ITH and were excluded in FST calculations. Both the primary tumor (median F_{ST}=0.180, range 0.150-0.430) and paired metastases (median F_{ST}=0.178, range 0.123-0.271) exhibited high F_{ST} values, consistent with rapid genetic diversification (FIG. 21). Proliferative indices based on Ki67 staining were also similar between paired CRCs and metastases (P=0.765, Wilcoxon Signed-Rank Test, FIG. 21).

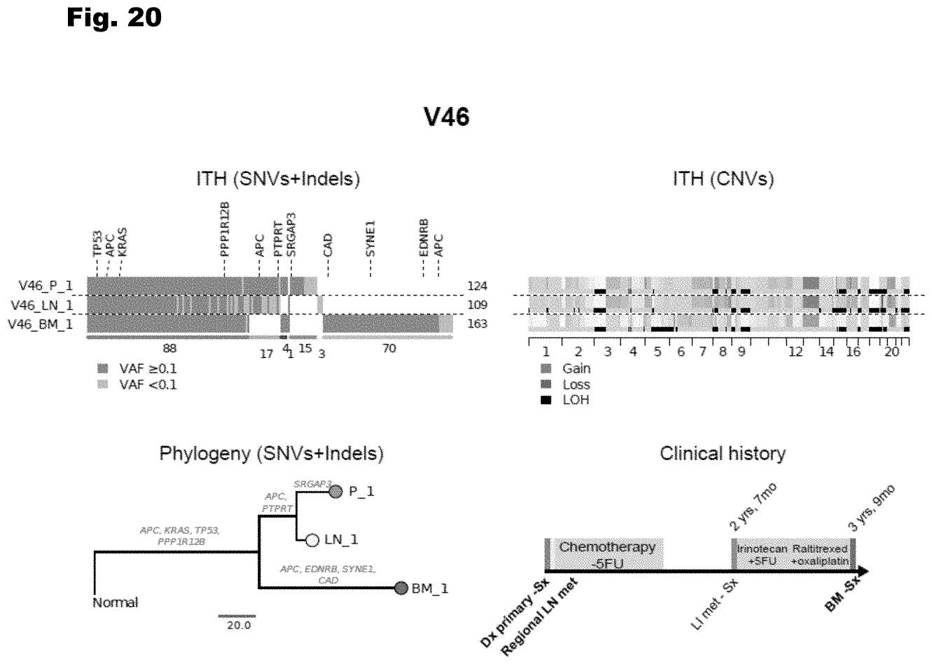

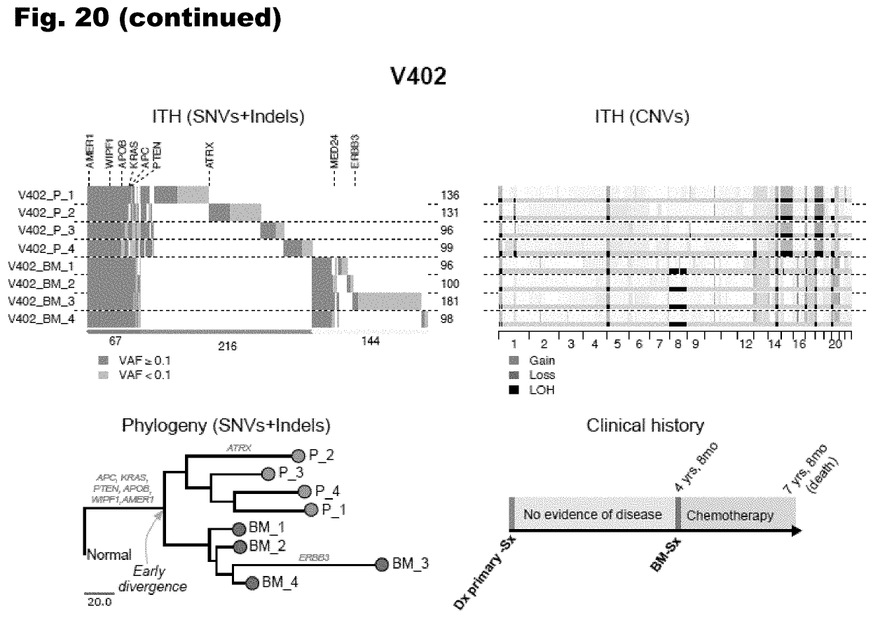

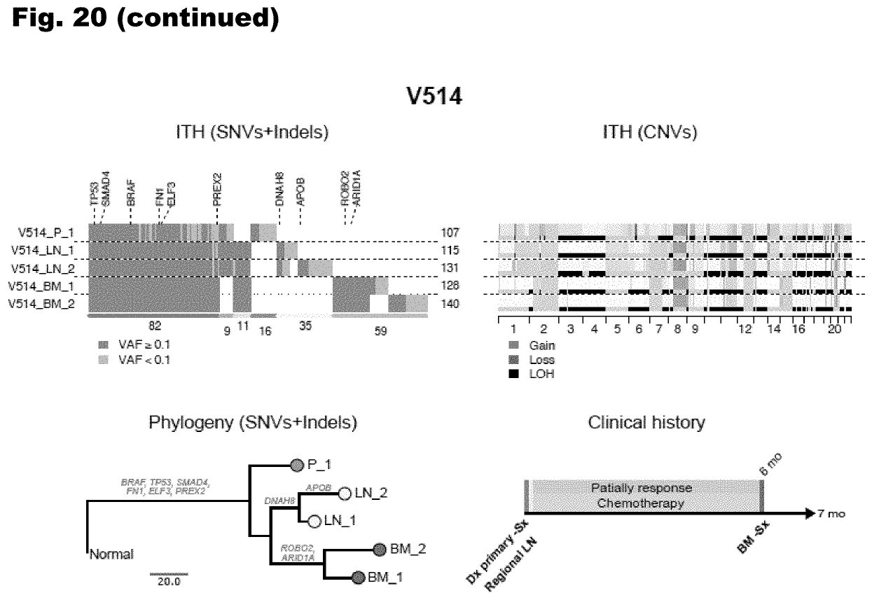

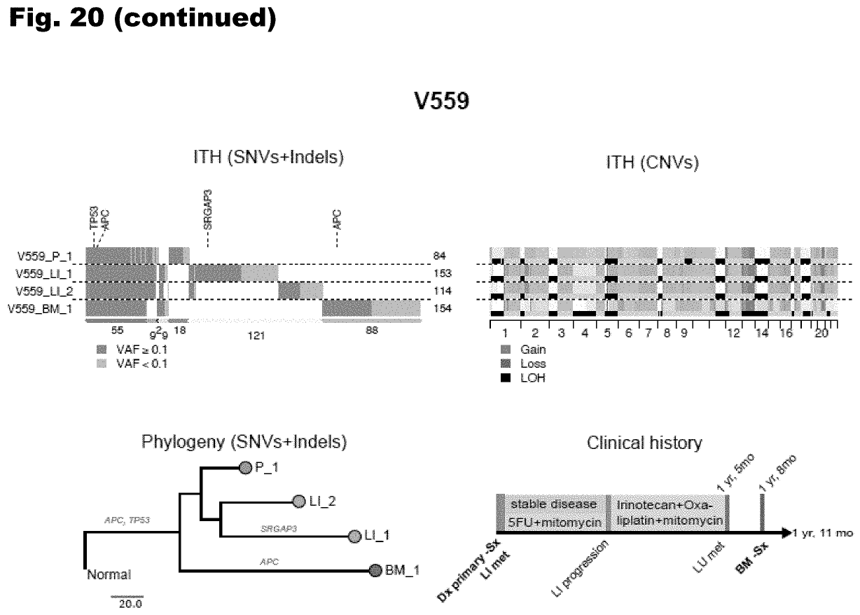

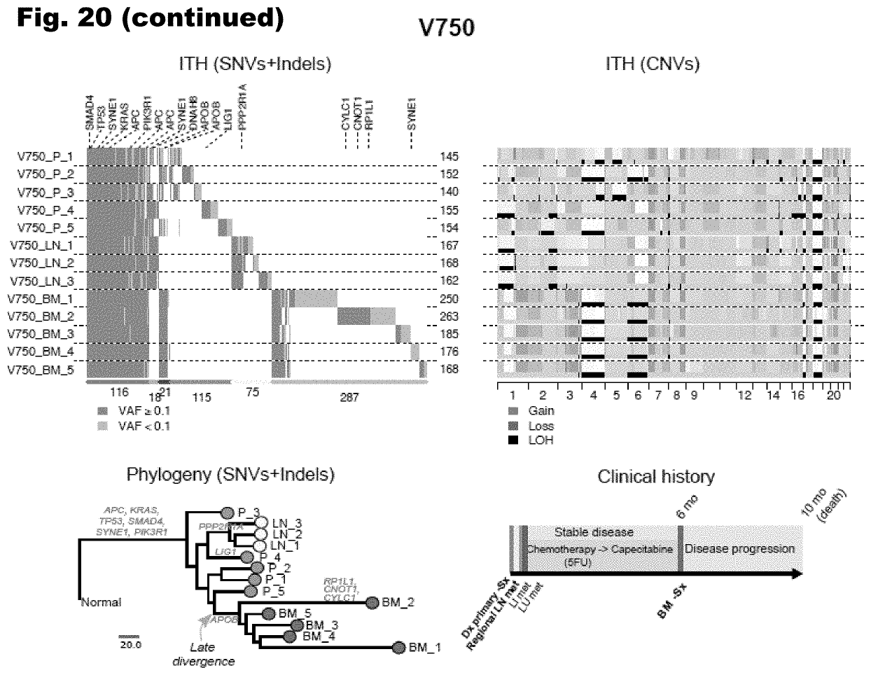

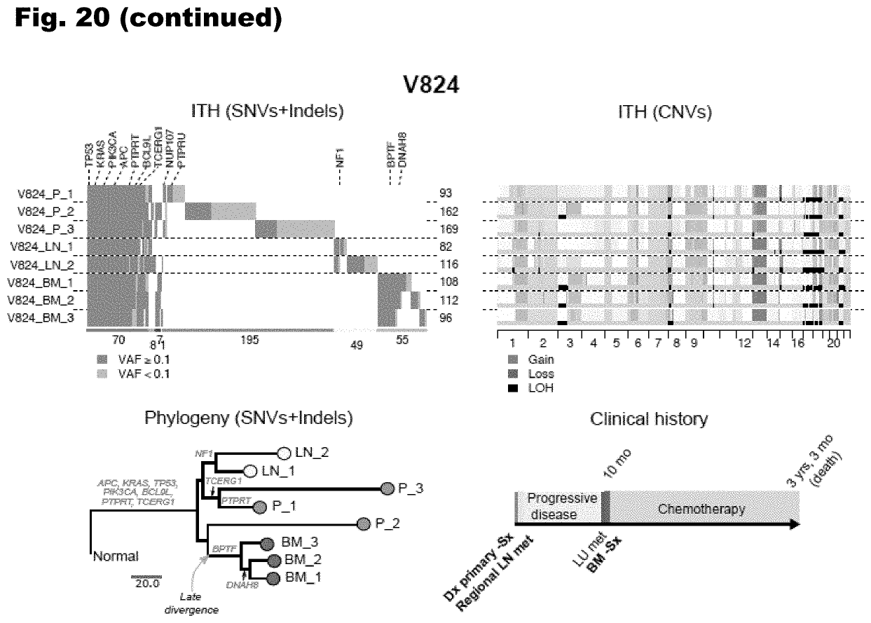

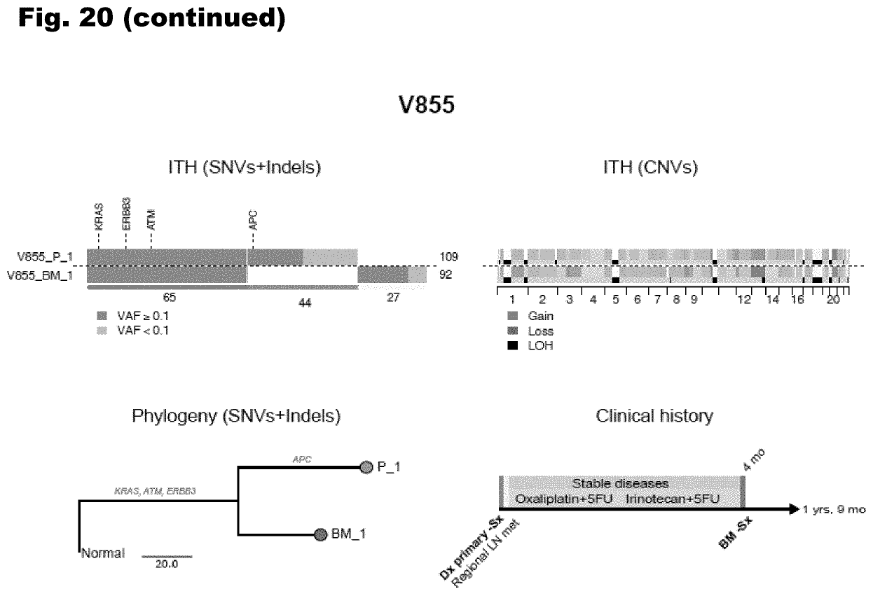

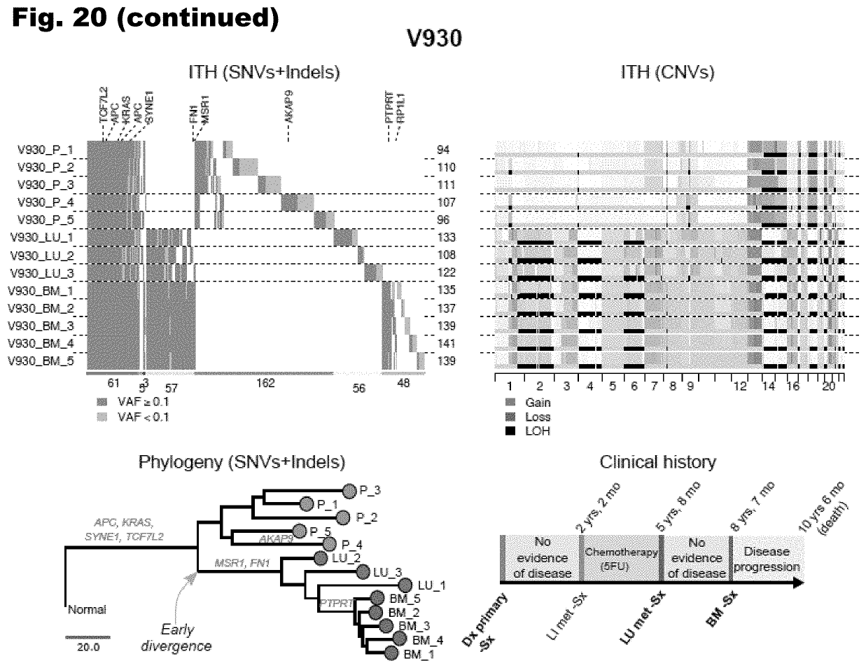

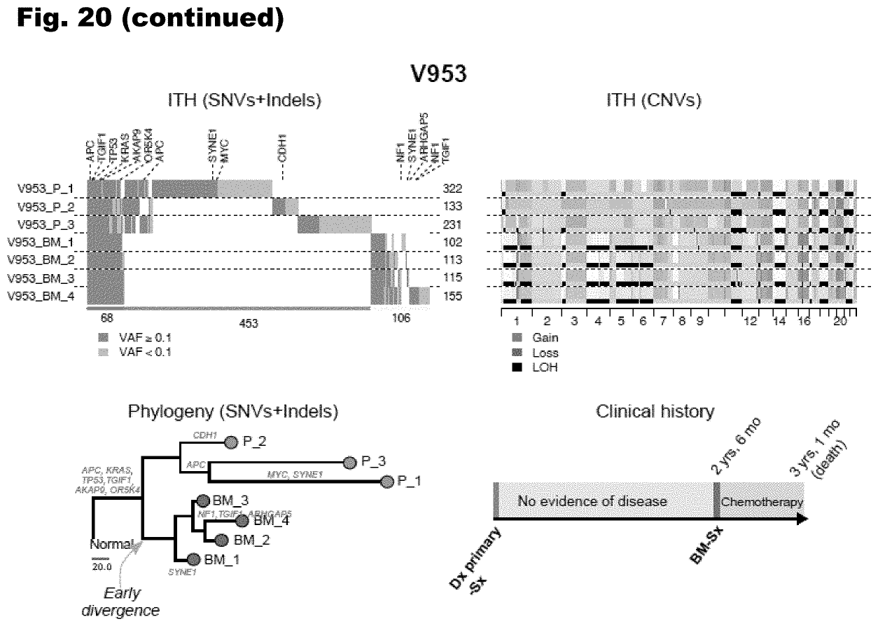

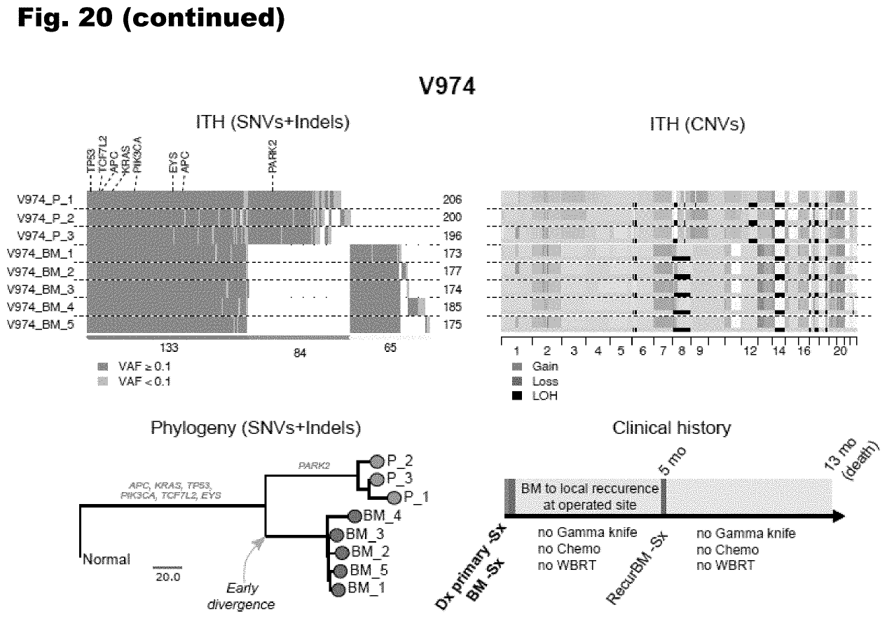

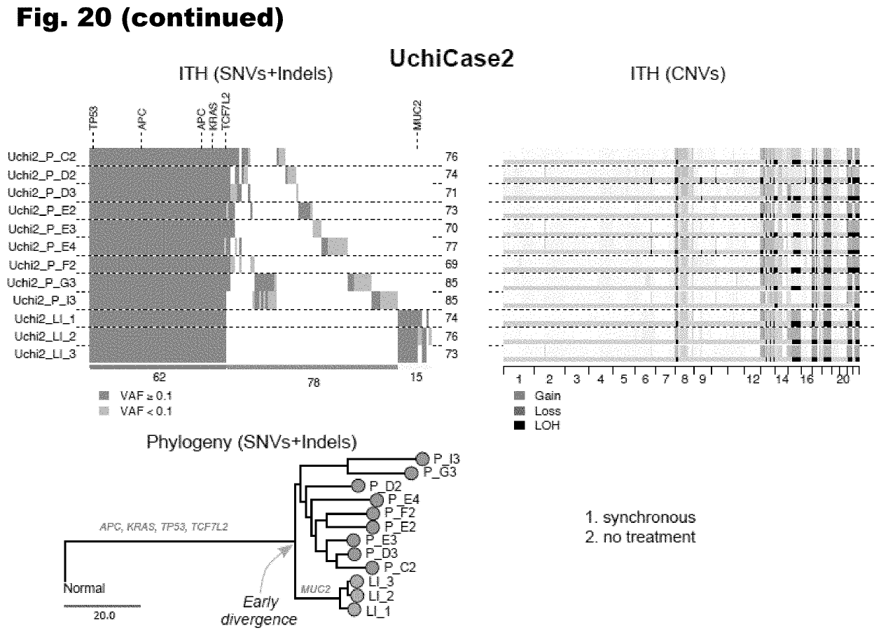

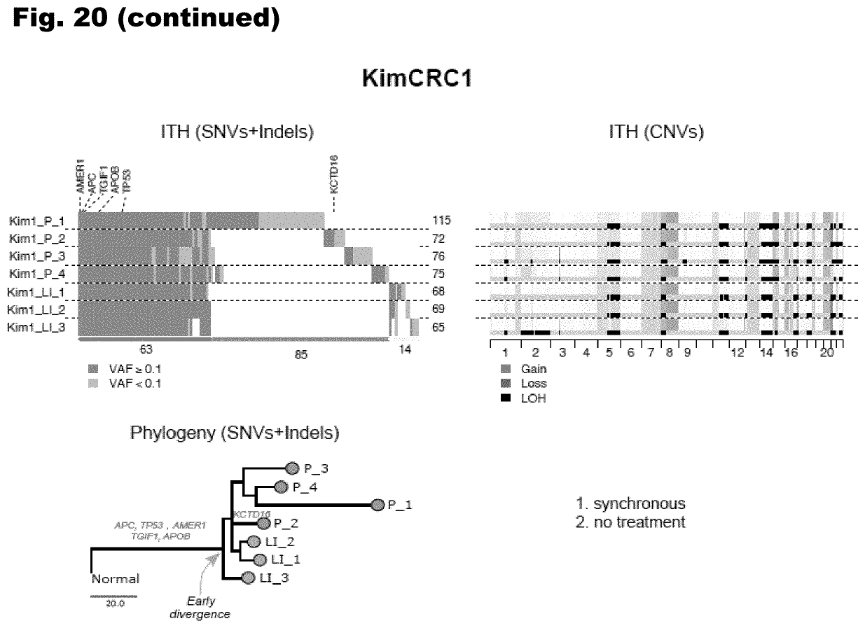

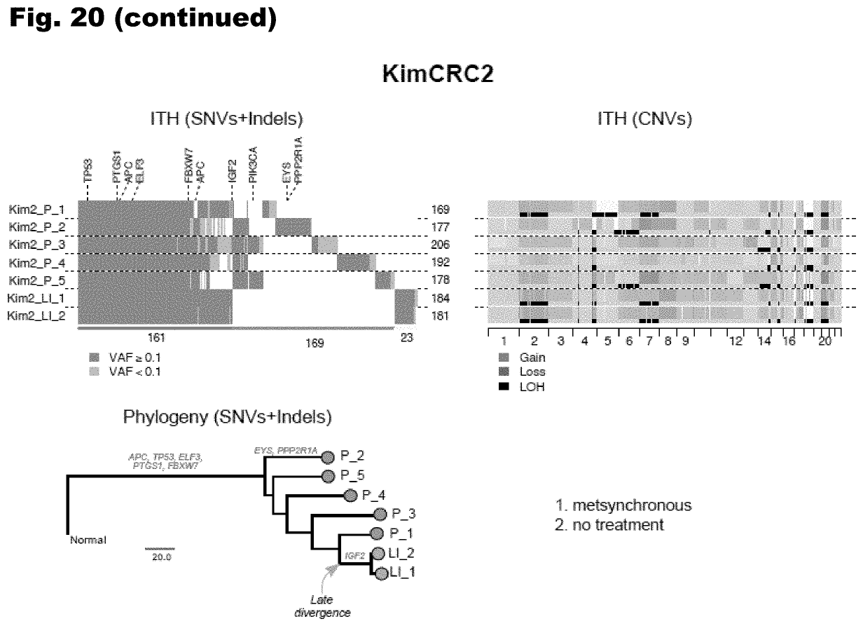

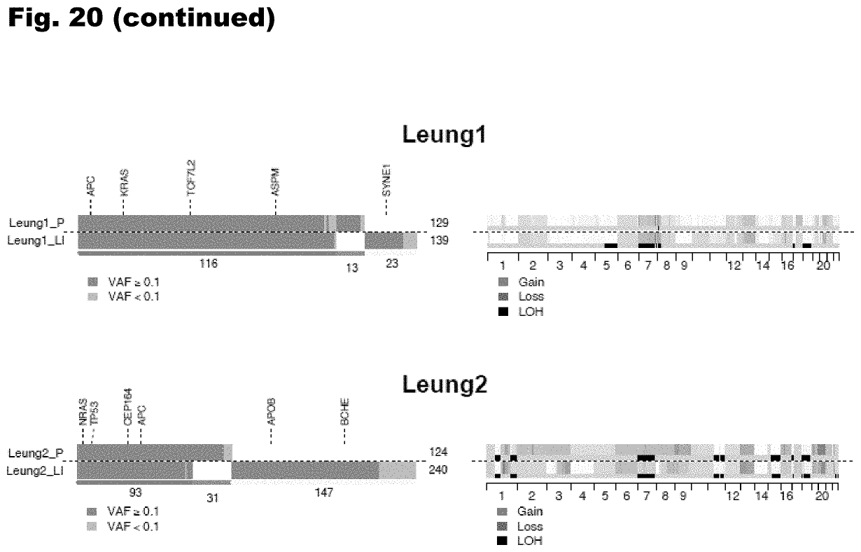

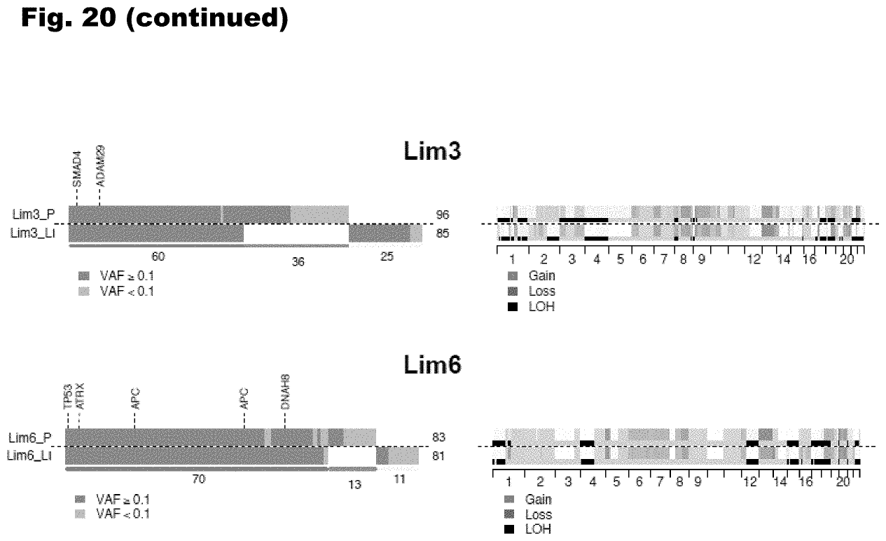

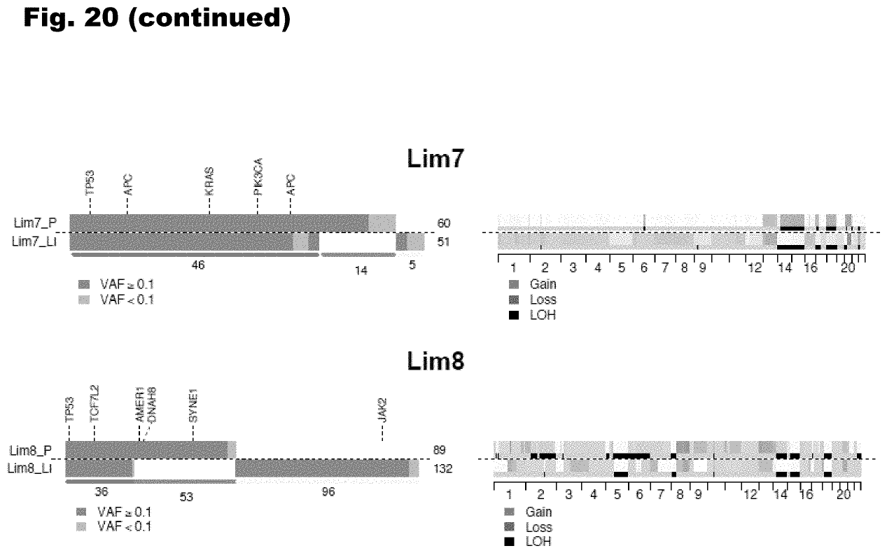

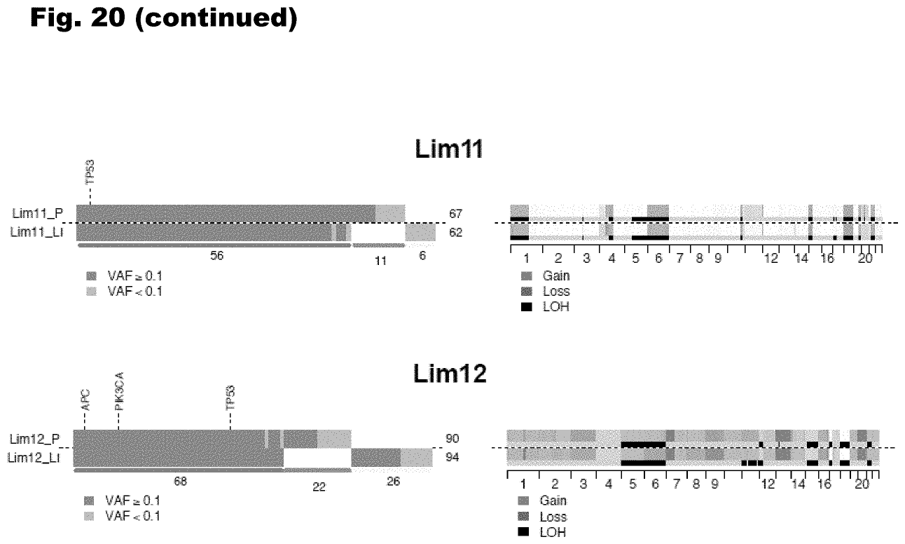

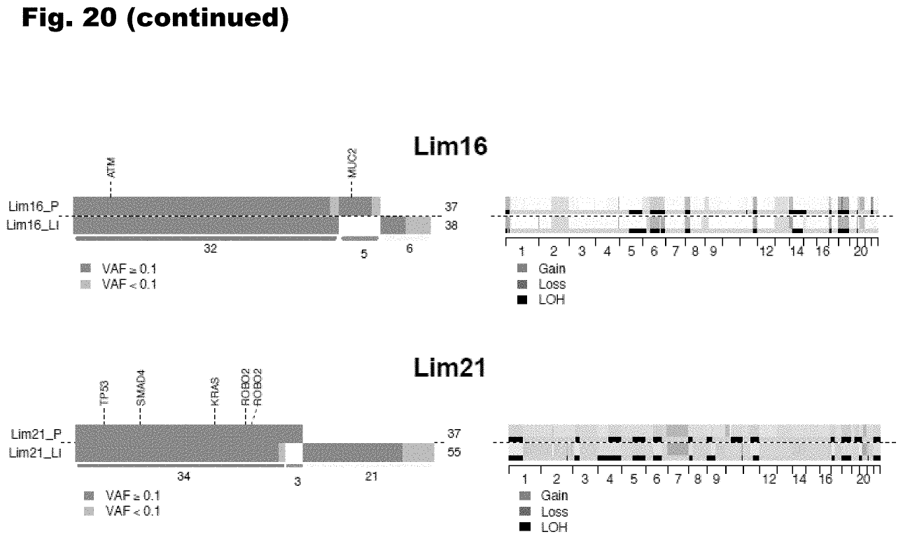

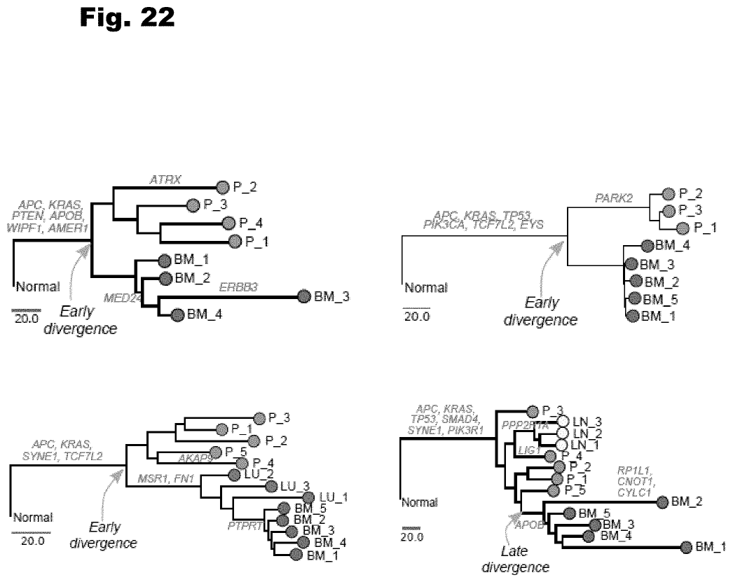

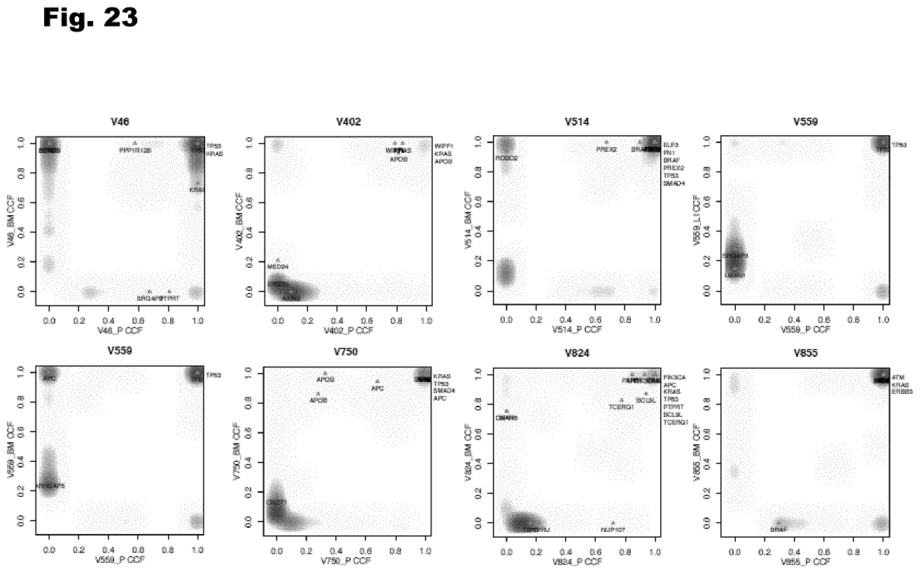

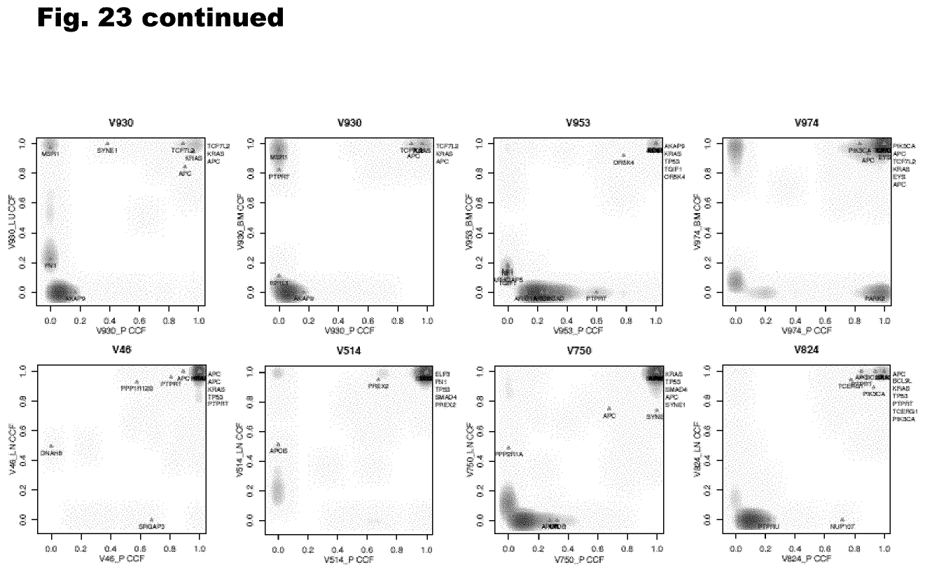

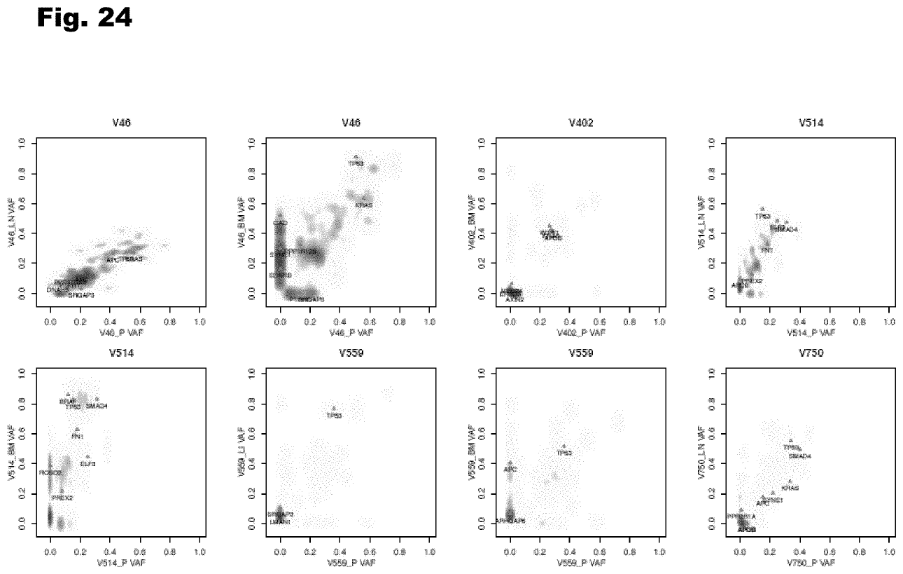

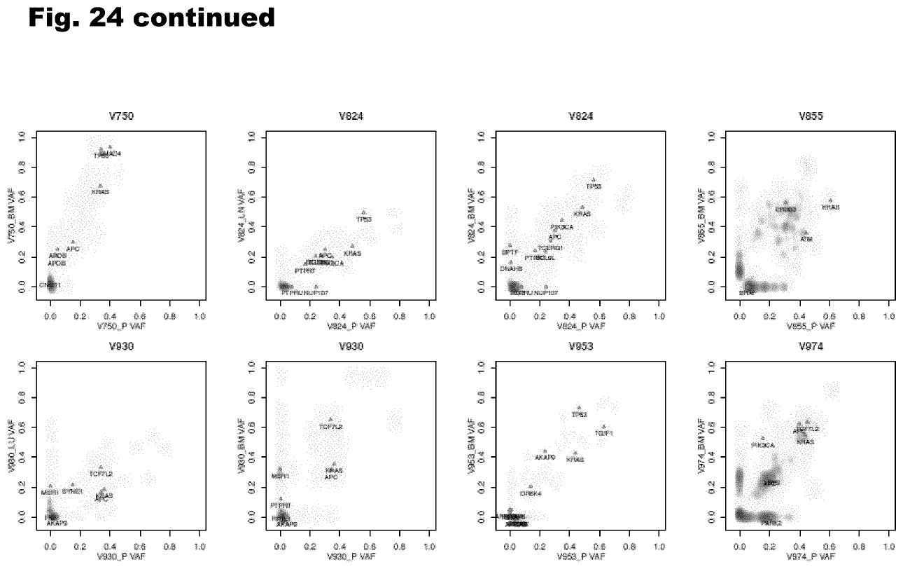

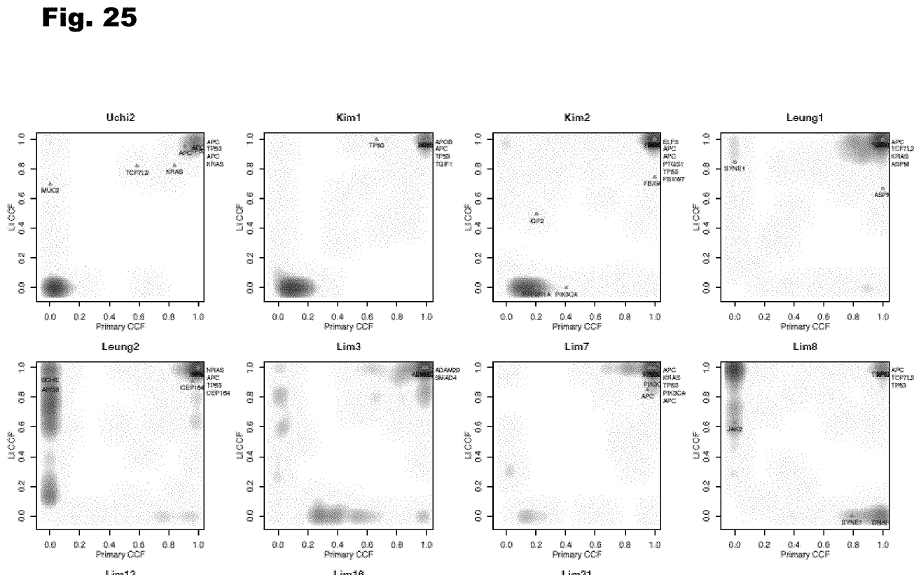

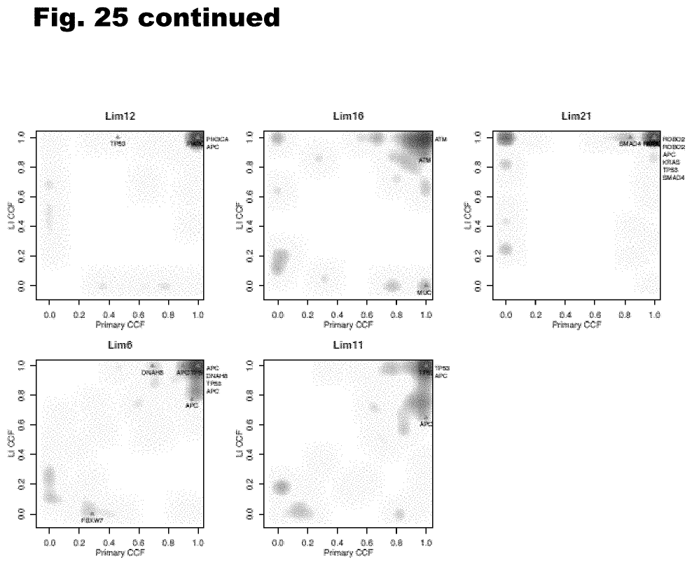

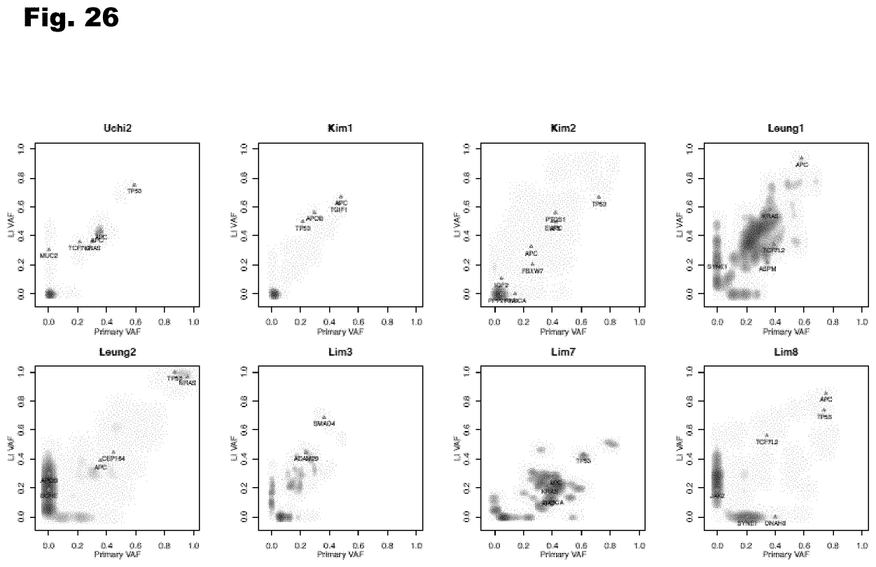

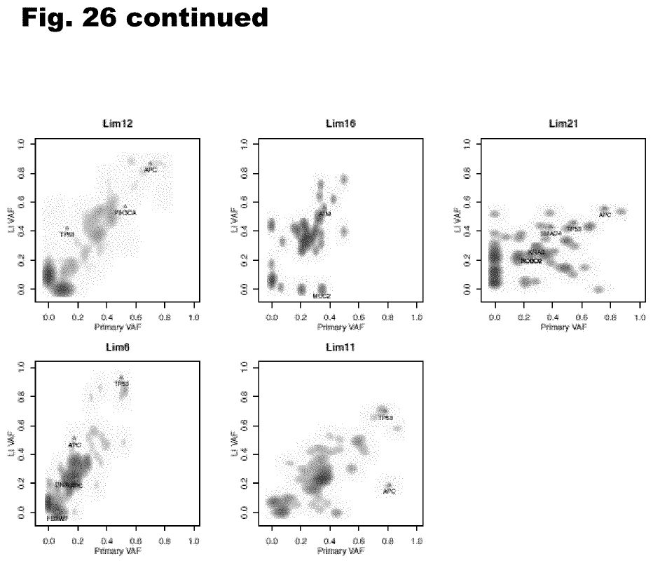

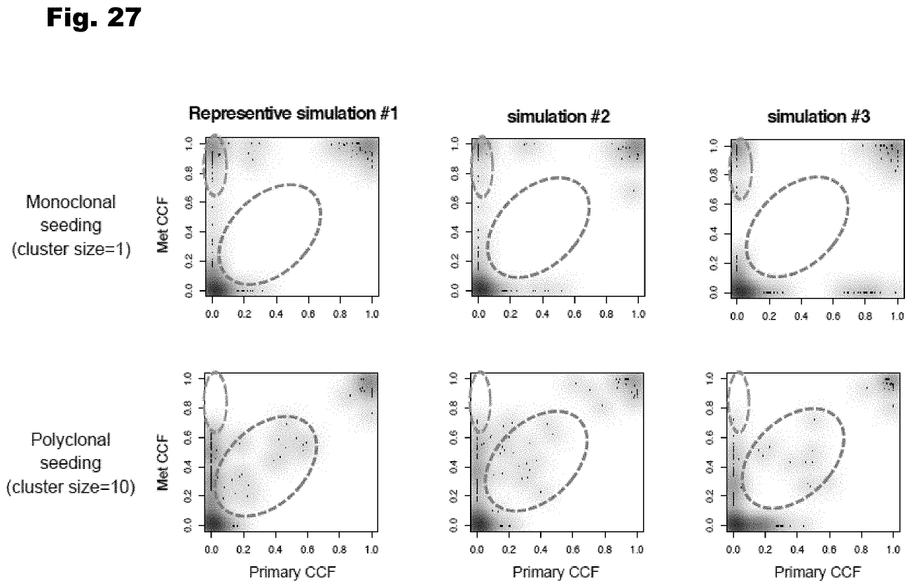

Tumor phylogenies were reconstructed using sSNVs and small insertions and deletions (indels) across multiple regions of each P/M pair using the maximum-parsimony method⁴⁵. Distant metastases corresponded to monophyletic clades in all but one (Kim1) case (8/9 with MRS) (FIGS. 20 and 22), consistent with the unique origin of the metastatic lineage. Inspection of the phylogeny for Kim1 indicated that the liver metastasis preceded the primary tumor, which is improbable and likely due to metastasis-specific loss of heterozygosity (LOH) spanning multiple mutations. In most patients, the metastatic lineage diverged prior to genetic diversification of the primary tumor (V402, V930, V953, V974, Uchi2; early divergence), whereas divergence occurred during diversification of the primary tumor in patients V750, V824 and Kim2 (late divergence). All brain metastases and most liver metastases harbored many private clonal sSNVs, but lacked shared subclonal sSNVs with the primary tumors, consistent with monoclonal seeding (FIGS. 23 to 26), as demonstrated by simulation studies (FIG. 27). Two liver metastases (Limb and Lim11), exhibited enrichment for shared subclonal mutations, but lacked metastasis-private clonal mutations, consistent with polyclonal seeding (FIGS. 25 to 27). These data suggest that distant metastases are often seeded by a single clone (a single cell or a group of genetically similar cells). Notably, the phylogenetic tree for case V930 indicates that the brain metastasis derived from the lung metastasis, in-line with the patient's clinical history (FIG. 18). Brain metastases and regional lymph node (LN) metastases formed separate clades in the two cases in which they were profiled (V750, V824), indicative of their independent clonal origin from primary tumor (FIGS. 20 and 22).

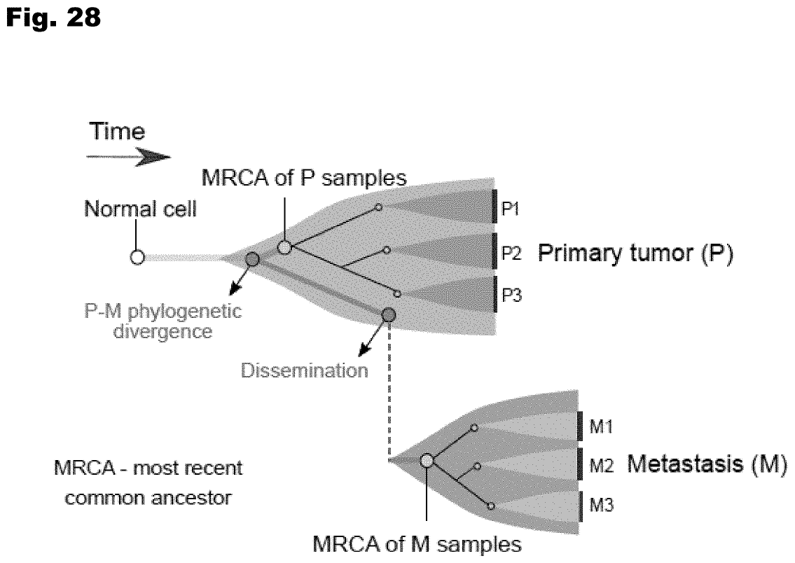

The finding that paired CRCs and metastases formed separate phylogenetic clades in most patients suggests that metastatic dissemination may occur early such that the primary tumor has sufficient time to accumulate many unique clonal mutations after dissemination. However, phylogenetic divergence may occur much earlier than dissemination (FIG. 28) and phylogenetics cannot resolve the timing of dissemination. As such, it was next sought to investigate the determinants of PMGD and to quantify the timing of metastasis.

### The Timing of Dissemination and P-M Genomic Divergence

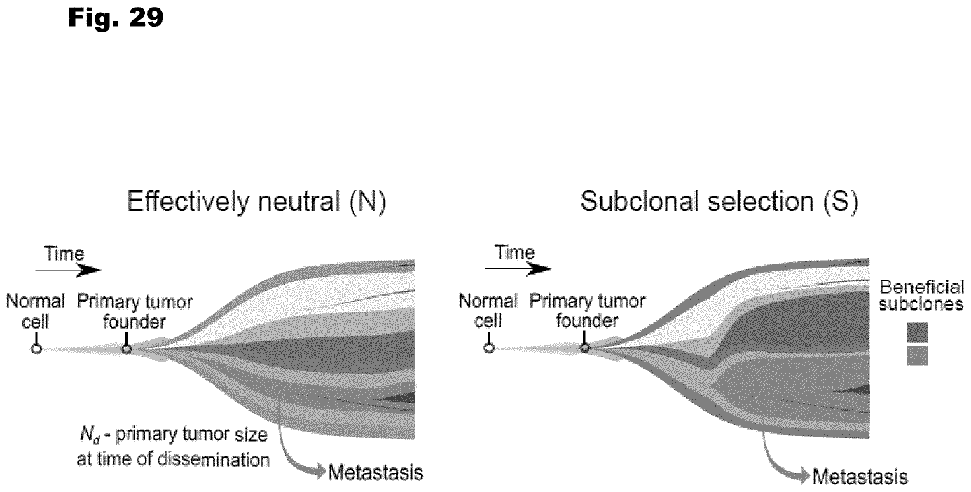

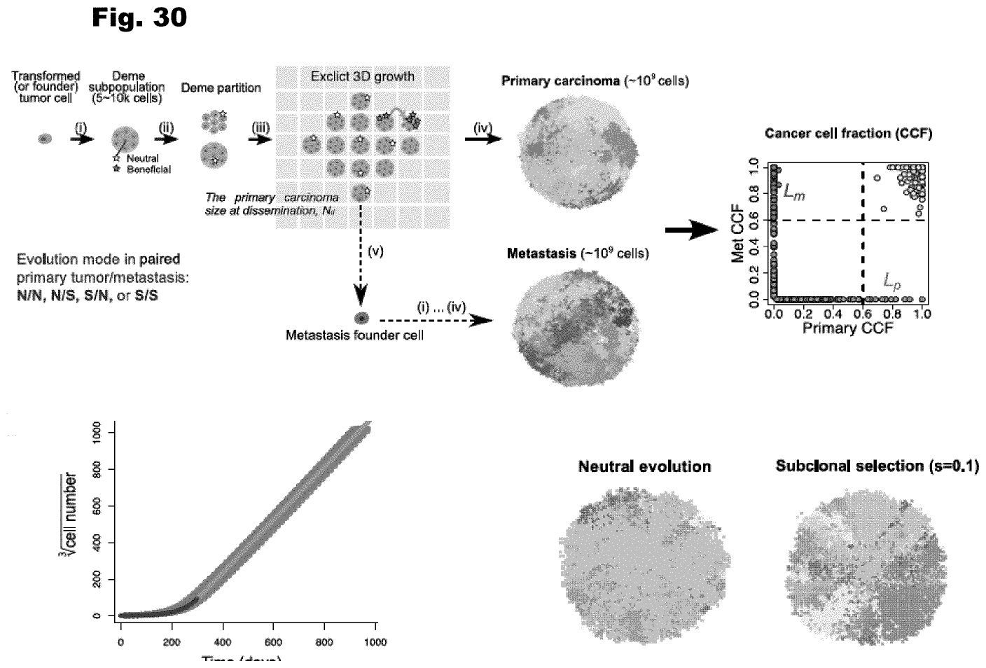

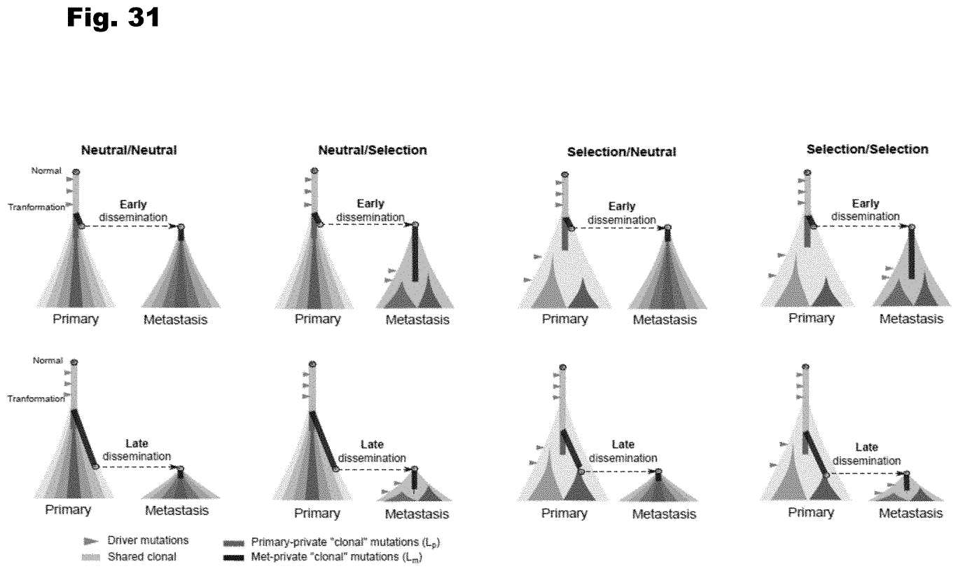

To model the evolutionary dynamics of metastasis, a 3-D agent-based computational model was developed to simulate the spatial growth, progression and lineage relationships of realistically sized patient tumors under varied parameters (FIGS. 29 and 30, Table 3). The growth of a primary CRC was modeled starting from a single founder cell and assume that the metastasis is seeded by a random single cell on periphery of primary tumor, yielding primary and metastatic tumors composed of ˜10⁹ cells (˜10 cm³). To account for distinct modes of tumor evolution, effective neutrality and stringent subclonal selection were simulated, resulting in four evolutionary scenarios for P/M pairs: Neutral/Neutral (N/N), Neutral/Selection (N/S), Selection/Neutral (S/N) and Selection/Selection (S/S) (see FIGS. 29 to 31). Using this simulation framework, where ground-truth values are known, the relationship between the number of M-private clonal sSNVs (Lₘ) and primary CRC size at the time of dissemination (N_{d}) was evaluated in hundreds of virtual paired P/M tumors, where size is a surrogate for time since cell division rates are unknown.

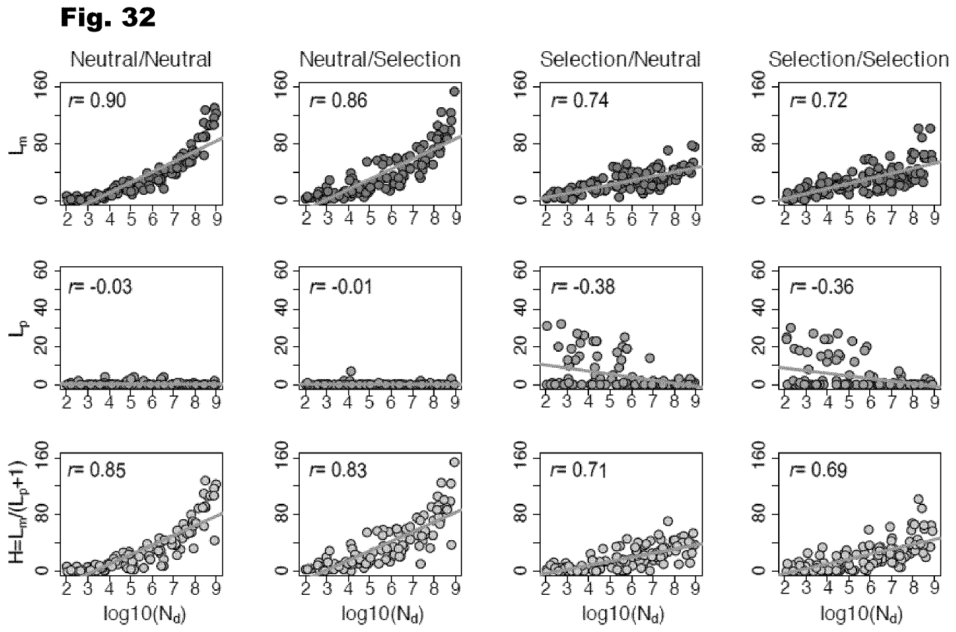

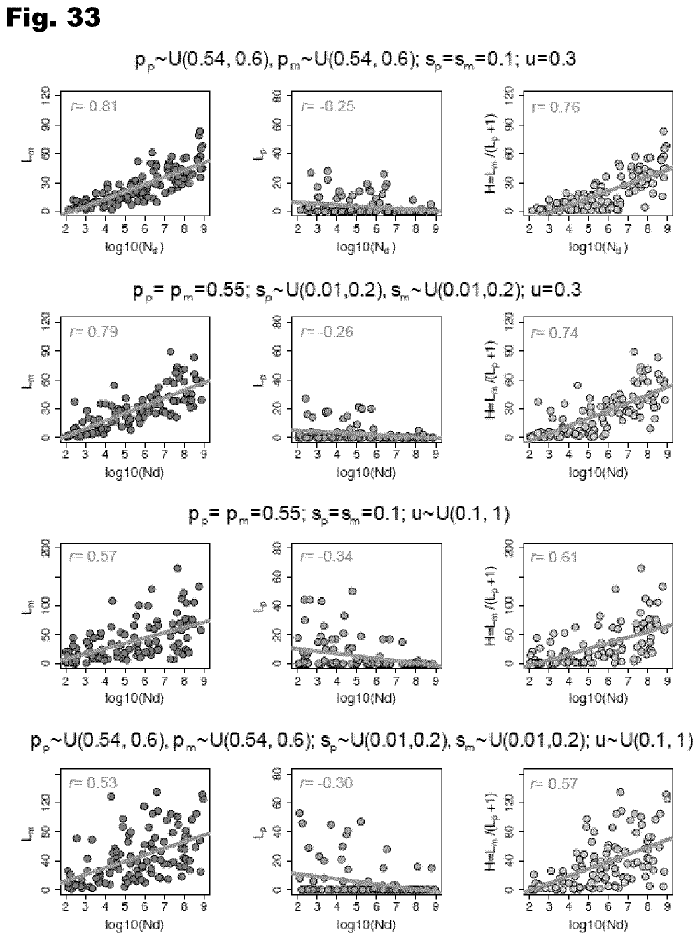

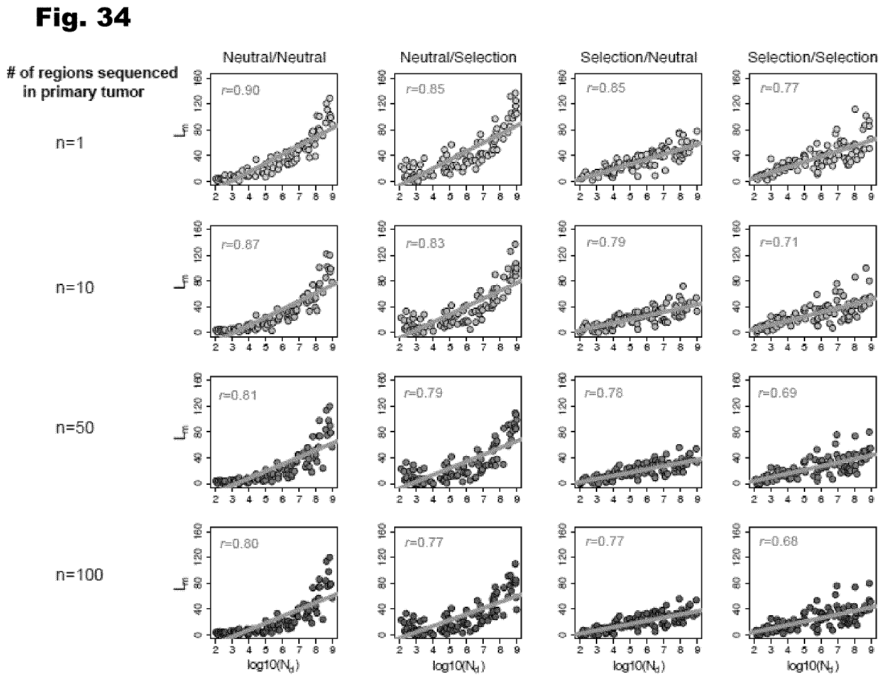

To define Lₘ, M-private clonal sSNVs were evaluated with respect to relatively high-frequency sSNVs in the whole primary tumor (CCF>1% ). Thus any clonal sSNV in the metastasis will be M-private if the CCF<1% in the primary tumor. It was found that Lₘ is positively correlated with N_{d} under all four evolutionary scenarios (FIG. 32). The positive relationship between Lₘ and N_{d} remains significant when accounting for variation in mutation rate, cell birth/death rate and selection intensity during tumor growth (FIG. 33). Lₘ was next evaluated by simulating sequencing reads from variable numbers of primary tumor regions (n=1, 10, 50 or 100) while considering the whole metastasis as a bulk sample within our computational model. The positive correlation between Lₘ and N_{d} was highly significant under all sampling scenarios, pointing to the robustness of this observation (FIG. 34). As expected, smaller Lₘ was observed when a greater number of primary tumor regions were sequenced because fewer mutations were M-private (FIG. 34). Mathematical analysis of the special case of neutral evolution and exponential growth further demonstrates the positive relationship between Lₘ and N_{d} (see Eq. S6 in “Algorithm” section below).

These data suggest that later dissemination results in more clonal mutations in the metastasis, many of which are at low frequency in the primary tumor and often undetectable in bulk sequencing. Accordingly, later dissemination will give rise to more metastasis-private clonal mutations in real sequencing data, leading to higher PMGD. It should be noted that if sampling of the primary tumor was exhaustive or if the metastasis-founder (M-founder) clone could be traced, neither of which are generally practical for studies of human tumors, one would expect very small Lₘ values and no correlation between Lₘ and N_{d} since all mutations in the M-founder cell that accumulated during primary tumor growth would be captured. In contrast, the number of P-private clonal sSNVs (Lₚ) exhibited slightly negative correlation with N_{d} when CRCs grew under stringent selection (S/N or S/S), whereas under neutral evolution (N/N or N/S) regardless of the timing of dissemination (FIGS. 32 and 33).

Early dissemination was defined as N_{d}<10⁸ cells (˜1 cm³ in volume), the size at which CRCs are generally clinically detectable, and late dissemination as N_{d}≥10⁸ cells. To establish intuition for the relationship between PMGD and N_{d}, the relationship was defined H=Lₘ/(Lₚ+1). In the simulation studies, H was positively correlated with N_{d} FIGS. 32 and 33), indicating that larger H values are associated with later dissemination. Indeed, late dissemination typically results in large H (>20). The observation that most patients in the mCRC cohort exhibited small H values (median=2.4, range: 0.5-23.5) suggests that early dissemination may be relatively common. While H is strongly associated with the timing of dissemination, it does not capture all components of PMGD, including the mutation rate as this is cancelled out in the division of Lₘ over Lₚ. Additionally, variation in Lₚ due to differences in the mode of evolution and sampling bias contribute to noise in H. To account for these sources of variability while estimating the timing of dissemination in individual patients, a powerful statistical inference framework grounded in population genetic theory was utilized.

### Quantitative Evidence for Early Metastatic Seeding in CRC

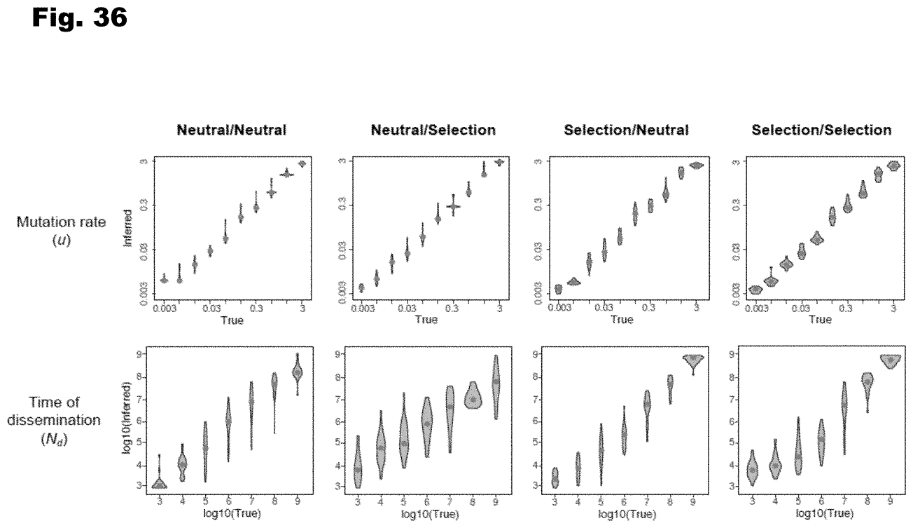

In order to infer the timing of dissemination N_{d}, mutation rate u (per cell division in exonic regions) and mode of tumor evolution in P/M pairs, SCIMET (Spatial Computational Inference of MEtastatic Timing) was developed, which couples the spatial (3D) agent-based model of tumor evolution with a statistical inference framework based on Approximate Bayesian Computation (ABC) (FIGS. 29-31 and 35, Tables 4 and 5). Since the patient genomic data were generally consistent with monoclonal seeding, it was assumed that a single cell seeds the metastasis (Limb and Lim11 were therefore excluded from this analysis). Evaluation of SCIMET on virtual tumors demonstrates the accurate recovery of the mutation rate and timing of dissemination (FIG. 36).

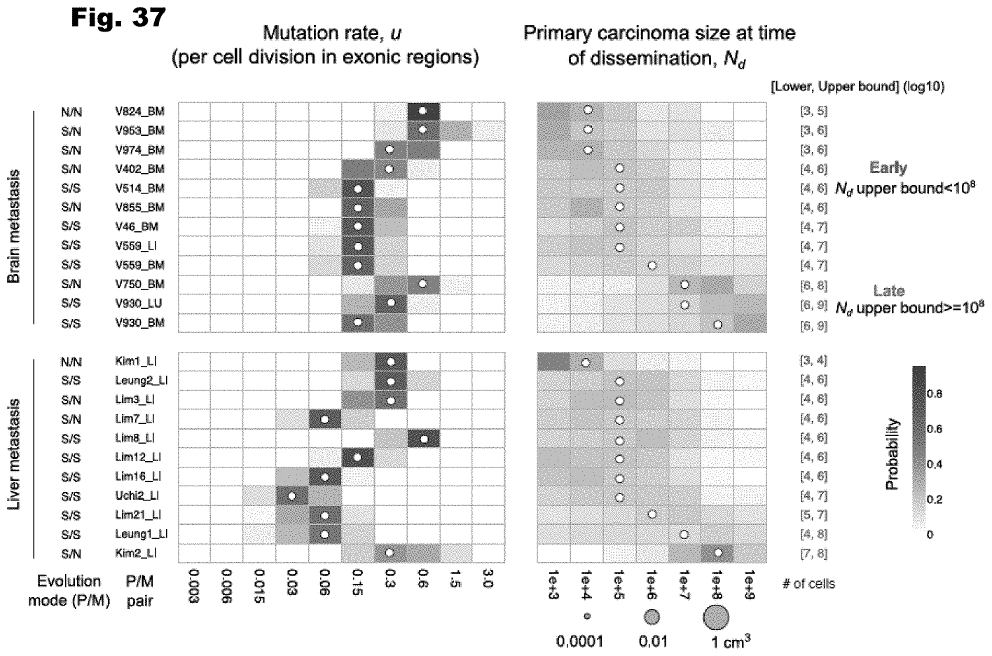

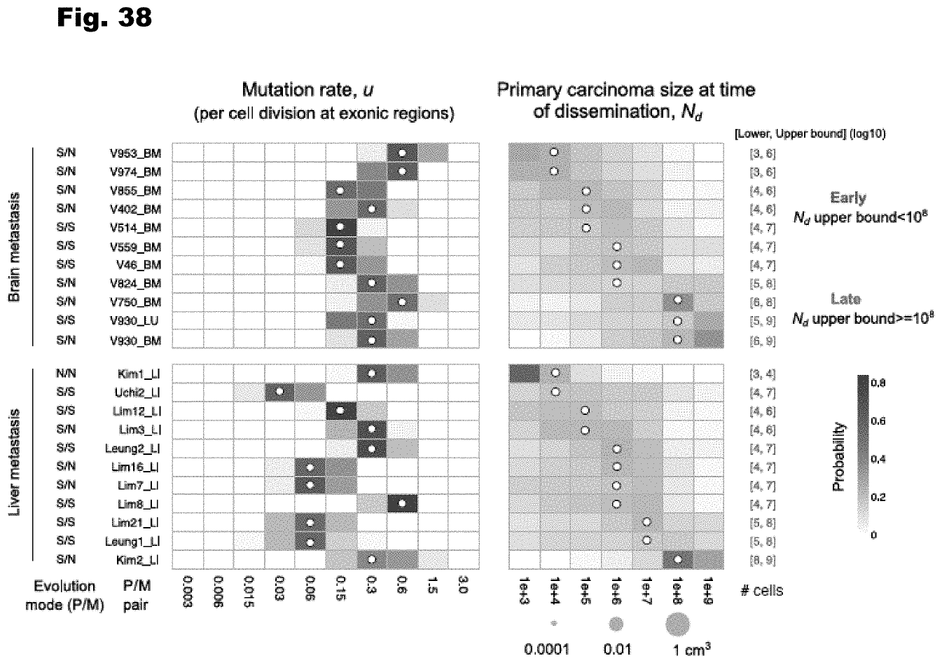

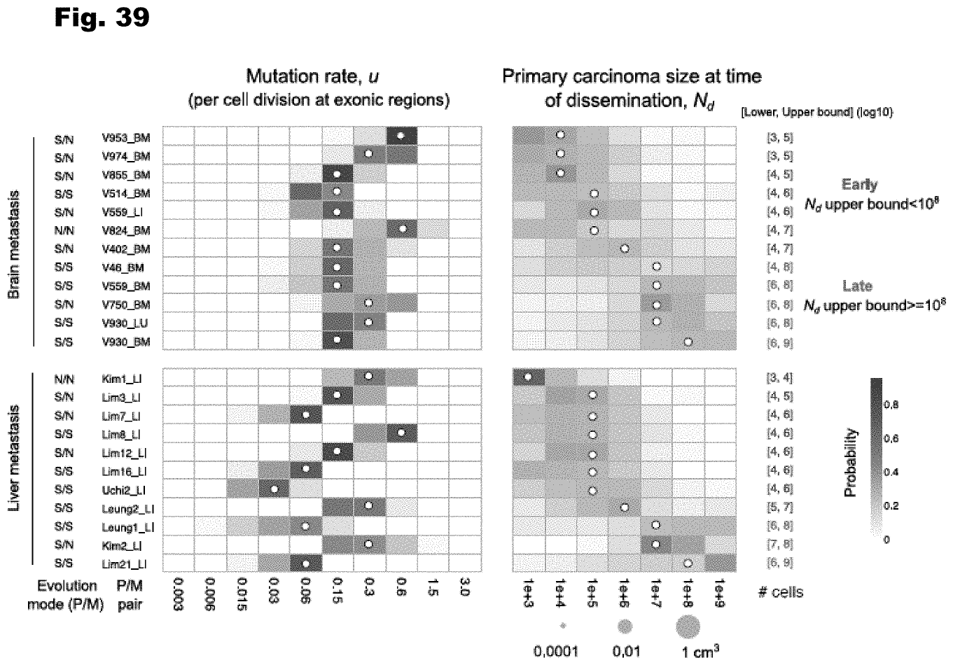

The majority (90%) of CRCs and metastases (57%) exhibited patterns consistent with subclonal selection (FIG. 37). Inference of patient-specific mutation rates via SCIMET showed an order of magnitude variation across patients (inferred u or ũ=0.06-0.6, corresponding to 10⁻⁹-10⁻⁸ mutations per base pair per cell division). Strikingly, in 83% (19/23) P/M pairs from 17/21 patients, dissemination was estimated to occur early when the primary CRC was below the limits of clinical detection (inferred N_{d} or Ñ_{d}<10⁸ cells) and typically when the primary tumor was composed of fewer than 10⁶ cells using conservative estimates (FIG. 37, Table 1). The inferred N_{d} values were also significantly smaller than the tumor size documented at the time of diagnosis in this cohort. Of note, early dissemination was common irrespective of the site of distant metastasis (8/10 brain, 10/12 liver, 1/1 lung). Congruent results were also obtained when accounting for higher ratios of cell birth/death rates in the primary CRC and metastasis (FIG. 38), the collective dissemination of small clusters of cells (n=10 cells) (FIG. 39) or single-region sampling (FIG. 40). Amongst the four cases where late dissemination was inferred, three had MRS data, enabling comparison with their phylogenies. For two patients (V750 brain metastasis and Kim2 liver metastasis) late dissemination was consistent with the tumor phylogeny (FIGS. 12 and 14). For patient V930, late dissemination was inferred for both the lung and brain metastases, consistent with the large H values (brain: H=23.5; lung: H=11). However, the tumor phylogeny indicates early divergence of the metastatic lineage (FIG. 12). This case illustrates that phylogenetic divergence can occur before dissemination (FIG. 14), emphasizing the need for a quantitative evolutionary framework to ‘time’ metastasis.

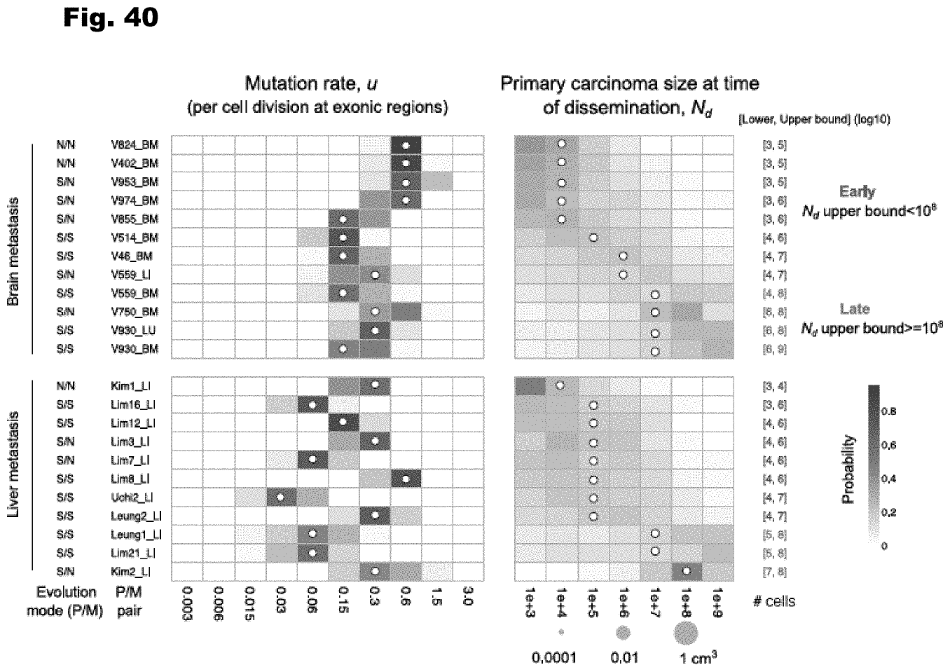

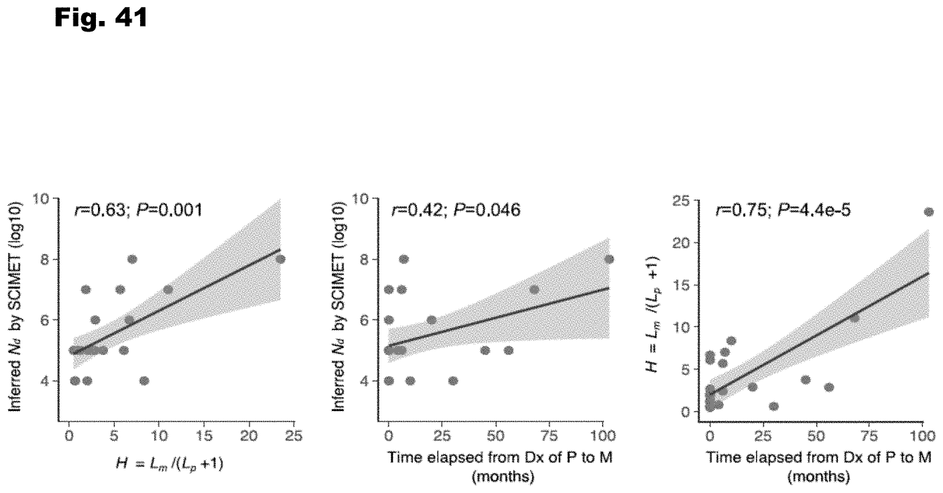

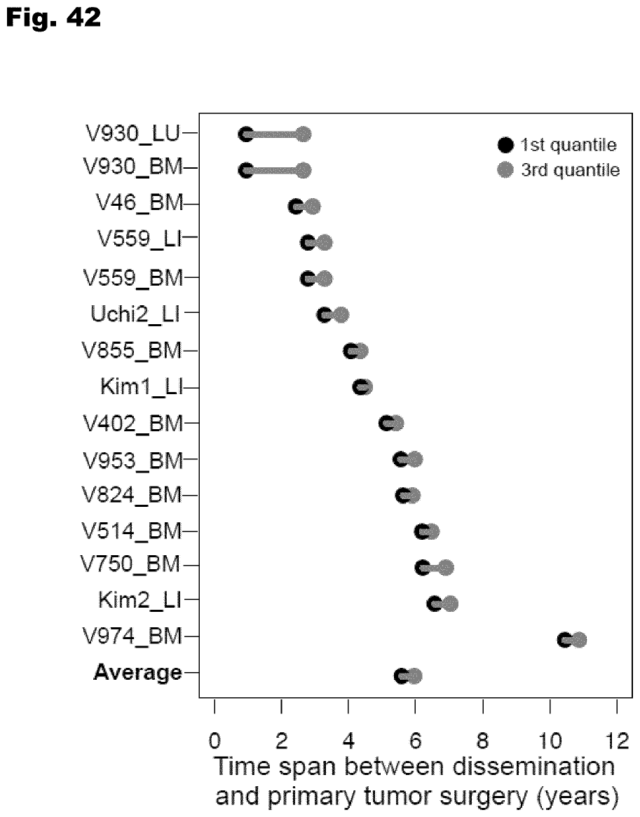

The inferred Ñ_{d} values based on SCIMET were positively correlated with H (Pearson's r=0.63, P=0.001, FIG. 41), consistent with the observation that the H metric reflects the timing of dissemination. Additionally, both Ñ_{d} and H were positively correlated with the time elapsed between diagnosis of the primary CRC and distant metastasis (FIG. 41), implying that metastases that are diagnosed later likely disseminated later. Further, it was estimated the time span between metastatic dissemination and surgical resection of the primary tumor by employing an approximate analytical function for our spatial tumor growth model and find that dissemination often occurred more than 3 years before surgery (FIG. 42).

### Metastasis-Associated Early Driver Gene Modules

As noted above, most canonical drivers were clonal and shared between paired primaries and metastases, indicative of their early acquisition before transformation. Taken this together with the finding that cancer cells seed metastases early in the majority of mCRCs in this cohort, specific combinations of early driver genes (modules) may confer metastatic competence. In support of this view, oncogene engineering of four canonical early driver genes (APC, KRAS, TP53, SMAD4) in wild-type primary colon organoids yielded metastases upon xenotransplantation (see A. Fumagalli, et al., Proc Natl Acad Sci USA 114, E2357-E2364 (2017), the disclosure of which is herein incorporated by reference). Similarly, in a mouse model of CRC, oncogenic Kras in combination with Apc and Trp53 deficiency was sufficient to drive metastasis (see A. T. Boutin, et al., Genes Dev 31, 370-382 (2017), the disclosure of which is herein incorporated by reference).

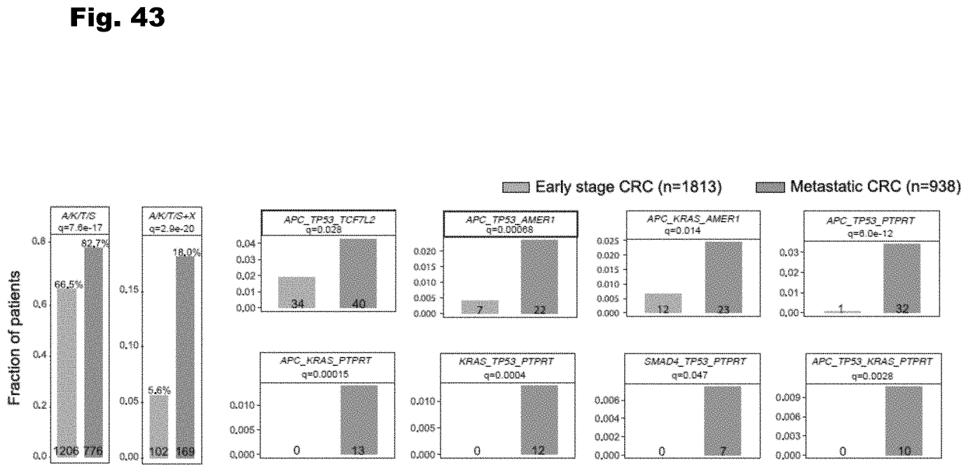

The association between the early driver modules defined in the mCRC cohort and metastatic proclivity was evaluated by analyzing a collection of 2,751 CRC patients, including 938 with metastatic disease (stage IV) and 1,813 early-stage (stage I-III) CRC patients that were prospectively sequenced as part of the MSK-Impact and GENIE studies. Strikingly, it was found that numerous early driver gene modules were significantly enriched in metastatic relative to early stage CRCs in this independent dataset after correction for multiple hypothesis testing (FIGS. 43, 44 and 45). These modules consist of a backbone of canonical ‘core’ CRC drivers (combinations of APC, KRAS, TP53 or SMAD4, abbreviated A/K/T/S) with one additional candidate metastasis driver (TCF7L2, AMER1 or PTPRT). Collectively, the ‘core’ modules plus an additional candidate metastasis driver shows a statistically significant enrichment in metastatic versus early stage CRCs (18% vs. 5.6%, respectively, q=2.9×10⁻²⁰). Examination of the prevalence and enrichment of individual modules indicates that PTPRT mutations in combination with canonical drivers were almost exclusively observed in metastatic patients (FIGS. 43, 44 and 45). Thus, PTPRT appears to be a highly specific driver of metastasis. PTPRT mutations were previously reported in 26% of colorectal cancers and loss of PTPRT in CRC and in head and neck squamous cell cancers results in increased STAT3 activation and cellular survival (see Z. Wang, et al., Science 304, 1164-6 (2004); X. Zhang, et al., Proc Natl Acad Sci USA 104, 4060-4 (2007); the disclosure of which are each herein incorporated by reference. It is now proposed that PTPRT mutations are predictive biomarkers for STAT3 pathway inhibitors, illuminating new therapeutic opportunities that target this pathway. Other modules involving AMER1 and TCF7L2 were also significantly enriched in metastatic cases, but were less specific perhaps because an additional driver defines the module. Thus we identify a compendium of metastasis driver modules that can inform the stratification and therapeutic targeting of patients with aggressive disease.

### Summary of Findings

As described herein, a novel theoretical and analytical framework was developed. The framework yields quantitative in vivo measurement of the dynamics of metastasis in a patient-specific manner, while accounting for confounding factors, including the founder event, the mode of tumor evolution, mutation rate variation and tissue sampling bias. By analyzing genomic data from paired primary CRCs and distant metastases to the liver and brain from five patient cohorts within this evolutionary framework, it was demonstrated that metastatic seeding often occurs early (17/21 patients), when the carcinoma is clinically undetectable (˜10⁴-10⁸ cells or 0.0001-1 cm³) and years before diagnosis and surgery (see FIGS. 37 to 42). The observation that early metastatic seeding was prevalent irrespective of the site of distant metastasis, suggests the generalizability of these results. Moreover, dissemination was early even when considering liver-exclusive and brain-exclusive metastases, which represent extremes in terms of their prevalence and prognosis. Collectively, these finding indicate that CRCs can be “born to be bad,” wherein invasive and metastatic potential is specified early. This finding illuminates the need to target the canonical drivers of tumorigenesis in therapy. However, not all tumors will metastasize and thus identifying biomarkers associated with aggressive disease will stratify therapeutic interventions.

Towards this end, metastasis-associated driver modules were validated in an independent cohort, thereby defining the molecular features of metastasizing clones. The overlap with drivers of initiation and combinatorial structure of these modules may explain why few drivers of metastasis have been identified to date. While the canonical driver landscape is relatively sparse, there are nonetheless many possible combinations of mutations that collectively disrupt key signaling pathways (WNT, TP53, TGFB, EGFR and cellular adhesion) enabling niche independence and outgrowth at foreign sites.

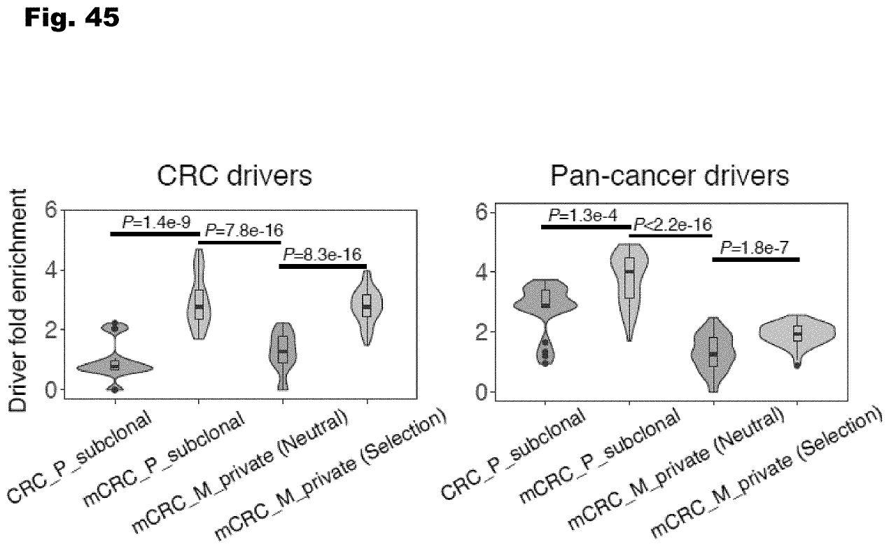

Of note, the vast majority (90%) of primary tumors in the mCRC cohort exhibited subclonal selection consistent with the metastatic clone having a selective growth advantage (FIG. 43). In contrast, a smaller proportion of early stage (I-III) CRCs (33%) exhibited patterns consistent with subclonal selection, suggesting that the mode of tumor evolution may correlate with disease stage or aggressiveness, although larger studies are needed to investigate this trend. Whereas drivers were not enriched in metastases when all cases were considered (FIG. 12), stratifying by the mode of tumor evolution revealed the enrichment of private high-frequency (CCF>20%) driver mutations in metastases evolving under stringent selection compared to those evolving neutrally (FIG. 45), implying that further subclonal driver mutations may occur during the growth of some metastases. Nonetheless, a sizeable proportion (43%) of distant metastases evolved neutrally, potentially reflecting the high fitness of the metastatic clone, consistent with a fitness plateau.

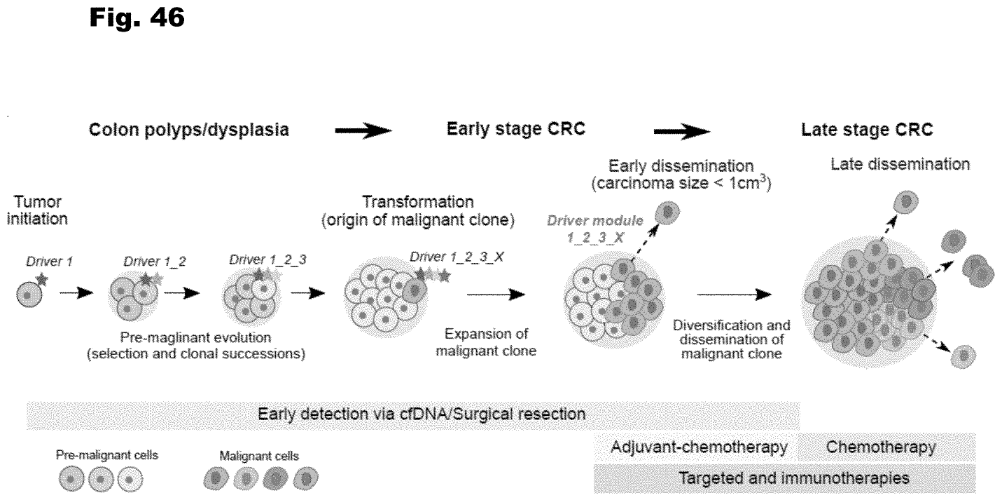

The finding that early dissemination resulting in successful metastatic seeding can occur before the primary tumor is clinically detectable in the majority (80%) of mCRC patients in this cohort underscores the importance of detecting malignancy at the earliest possible stage (FIG. 46). Such small tumors fall below the detection limits for current imaging modalities, but advances in profiling circulating cell-free tumor DNA may ultimately enable earlier non-invasive detection. Importantly, a considerable number of mCRC patients did not exhibit early systemic spread, suggesting that colonoscopy can be beneficial in this subgroup. Our data also suggest that early-stage patients harboring combinations of driver genes that confer a high risk of metastasis would particularly benefit from adjuvant chemotherapy to target micro-metastatic disease.

### Methods

### Clinical Specimens, Pathology Review and Sequencing Studies

Briefly, archived formalin-fixed paraffin-embedded (FFPE) tissue specimens from 10 patients with metastatic CRC, including primary tumor, matched metastases and adjacent normal colon tissue, were obtained from the Medical University of Vienna brain metastasis bio-bank, which was established in accordance with ethical guidelines (approval 078/2004). Tissue specimens were collected during the course of routine clinical care and clinical data were retrieved by retrospective chart review. All samples were de-identified and patients in the brain metastasis cohort were deceased prior to initiating this study. Brain metastases were available for all patients (BM, n=10) and for several patients metastases to the liver (LI, n=1), lung (LU, n=1), and regional lymph nodes (LN, n=4) were also available (Table 1). For 6 of the 10 patients, multiple specimens (n=3-5) from both the primary and metastasis were sampled and sequenced (Table 1). Histological sections were independently reviewed by expert pathologists (A.B, P.B, C.J.S). The Ki67 proliferative index was determined via immunohistochemical staining, as previously described (see A. S. Berghoff, et al., Neuropathol Appl Neurobiol 41, e41-55 (2015), the disclosure of which is herein incorporated by reference). Consistent with the growth of CRC brain metastases in an expansive rather than infiltrating fashion, no normal brain parenchyma was observed within the main brain metastasis lesion.

For all patients regions of high-cellularity (>60%) were selected for DNA isolation using the QIAamp DNA FFPE Tissue Kit (Qiagen). Libraries were prepared using the Agilent SureSelect Human All Exon kit or Ilumina Nextera Rapid Capture Exome (NCRE) kit for sequencing on the Illumina Hiseq 2000/2500 or Nextseq 500. Paired sequencing reads were aligned to human reference genome build hg19 with BWA (v.0.7.10) (H. Li and R. Durbin, Bioinformatics 25, 1754-60 (2009), the disclosure of which is herein incorporated by reference). Duplicate reads were flagged with Picard Tools (v.1.111). Aligned reads were further processed with GATK 3.4.0 for local re-alignment around insertions and deletions and base quality recalibration.

De-identified exome sequencing data from metastatic colorectal cancer patients in four published datasets (Uchi et al., Kim et al., Leung et al., and Lim et al., each of which cited supra) were also examined using the same unified bioinformatics framework detailed below. After excluding tumors with low purity (<0.4), 46 tumor specimens from 13 mCRC patients with paired liver metastases were retained and referred to this as the liver metastasis cohort.

### Somatic SNV Detection and Filtering

sSNVs were called by MuTect (v.1.1.7) with paired tumor and normal sequencing data. sSNVs failing MuTect's internal filters, having fewer than 10 total reads or 3 variant reads in the tumor sample, fewer than 10 reads in the normal sample, or mapping to paralogous genomic regions were removed (for more on MuTect, see K. Cibulskis, et al., Nat Biotechnol 31, 213-9 (2013), the disclosure of which is herein incorporated by reference). Additional Varscan (v.2.3.9) filters were applied to remove sSNVs with low average variant base qualities, low average mapping qualities among variant supporting reads, strand bias among variant supporting reads and high average mismatch base quality sums among variant supporting reads, either within each tumor sample or across all tumor samples from the same patient (for more on MuTect, see D. C. Koboldt, et al., Genome Res 22, 568-76 (2012), the disclosure of which is herein incorporated by reference). Additional filtering removed sSNVs detected in a panel of normals (PON) by running MuTect in single-sample mode with less stringent filtering criteria (artifact detection mode). sSNVs called in at least two normal samples were included in the PON sSNV list. For FFPE samples, sSNVs called in samples from one patient were checked against samples from all other patients to flag those that might be artifactual. The maximal observed variant allele frequencies (VAF) across all samples from each patient were calculated based on raw output files from MuTect. sSNVs with maximal observed VAFs between 0.01 and 0.05 in at least two other patients were removed. Small insertions and deletions (indels) were called with Strelka (v.1.0.14) and annotated by Annovar (v.20150617) (for more on Annovar, see K. Wang, M. Li, and H. Hakonarson, Nucleic Acids Res 38, e164 (2010), the disclosure of which is herein incorporated by reference). sSNVs and small insertions and deletions (indels) in protein coding regions were retained for downstream analyses. Additional filters were applied to exclude possible artifactual sSNVs due to the processing of FFPE specimens. Specifically, artifacts among C>T/G>A sSNVs with bias in read pair orientation were filtered in each individual FFPE sample, similar to the approach of Costello et al. (Nucleic Acids Res 41, e67 (2013), the disclosure of which is herein incorporated by reference).

For patients with MRS data, it was sought to exploit this information by retrieving read counts for sSNVs across samples from the same patient. To obtain depth and VAF information across all samples from the same patient, for each sSNV and in each tumor sample that an sSNV was not originally called in, the total reads and variant supporting reads were counted using the mpileup command in SAMtools (v.1.2) (for more on SAMtools, see H. Li, et al., Bioinformatics 25, 2078-9 (2009), the disclosure of which is herein incorporated by reference). Only reads with mapping quality≥40 and base quality at the sSNV locus≥20 were counted and used to calculate VAF values for that sSNV.

### Copy-Number Analysis, Tumor Purity and CCF Estimation

Copy number analysis was performed using TitanCNA (v.1.5.7) (for more on TitanCNA, see G. Ha, et al., Bioinformatics 25, 2078-9 (2009), the disclosure of which is herein incorporated by reference). Briefly, TitanCNA uses depth ratio and B-allele frequency information to estimate allele-specific absolute copy numbers with a hidden Markov model, and estimates tumor purity and clonal frequencies. Only autosomes were used in copy number analysis. First, for each patient, germline heterozygous SNP at dbSNP 138 loci were identified using SAMtools and SnpEff (v.3.6) in the normal sample. HMMcopy (v.0.99.0) was used to generate read counts for 1000-bp bins across the genome for all tumor samples (for more on HMMcopy, see G. Ha, et al., Genome Res 22, 1995-2007 (2012), the disclosure of which is herein incorporated by reference). Whole-exome sequences (WES) from multiple normal samples per patient were pooled separately for the purpose of calculating read counts in the bins and the pooled normal read depth data were used as controls for the calculation of depth ratios only. TitanCNA was used to calculate allelic ratios at the germline heterozygous SNP loci in the tumor sample and depth ratios between the tumor sample and the pooled normal data in bins containing those SNP loci. Only SNP loci within WES covered regions were then used to estimate allele-specific absolute copy number profiles. TitanCNA was run with different numbers of clones (n=1-3). One run was chosen for each tumor sample based on visual inspection of fitted results, with preference given to the results with a single clone unless results with multiple clones had visibly better fit to the data. Results from tumor samples from the same patient were inspected together to ensure consistency. Overall ploidy and purity for each tumor sample was calculated from the TitanCNA results. For the public datasets including liver-exclusive mCRCs, cases with estimated purity >0.4 in both the primary tumor and paired metastases (FIG. 7) were included since low purity hinders accurate SNV/CNA calling.

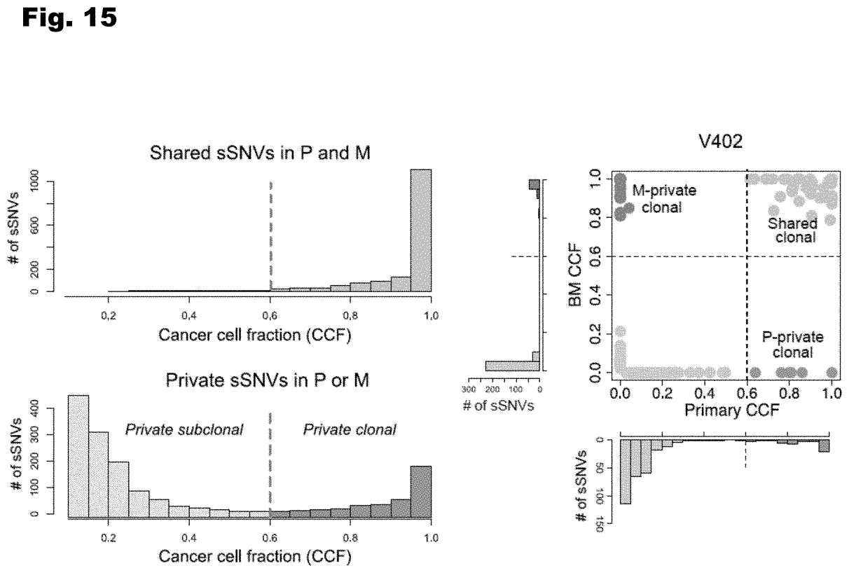

Mutational cancer cell fractions (CCFs) were estimated with CHAT (v 1.0) (for more on CHAT, see B. Li and J. Z. Li, Genome Biol 15, 473 (2014), the disclosure of which is herein incorporated by reference). CHAT includes a function to estimate the CCF of each sSNV by adjusting its variant allele frequency (VAF) based on local allele-specific copy numbers at the sSNV locus. sSNV frequencies and copy number profiles estimated from previous steps were used to calculate CCFs for all sSNVs in autosomes (using a modified function). The CCFs were also adjusted for tumor purity. The merged CCF of each sSNV is computed by integrating CCFs from multiple regions when MRS data is available:

$$C C F = \{ \begin{matrix}\frac{{Σ}_{i = 1}^{k} C C {F}_{i} \times {d}_{i}}{{Σ}_{i = 1}^{k} {d}_{i}} , & C C F < 1 \\ 1 , & C C F \geq 1\end{matrix} \qquad EQ . 1$$

where dᵢ and CCFᵢ are the sequencing depth and cancer cell fraction estimation in region i, respectively. Of note, the vast majority (99%) of P-M shared sSNVs have CCF (or merged CCF)>60%, a cutoff that also optimally distinguishes the site-private clonal and subclonal sSNV clusters (FIG. 15). Thus 60% was used as the CCF cutoff to define clonal versus subclonal sSNVs in the primary-metastasis genomic divergence (PMGD) analysis.

### Data Processing for Downstream Analysis

For each tumor site (primary or metastasis) in a patient, the average CCF estimate of a sSNV is set to 0 if neither of these two criteria are met: a) VAF≥0.03 and variant read count≥3; b) VAF≥0.1 in any of the regions. The following additional filters were applied to summarize the MRS P/M data in a given patient:
1) Filter out sSNVs without VAF≥0.05 and variant read count≥3 or VAF≥0.1 in any samples from this pair of sites
2) Filter out sSNVs with total read depth<20 from either of the two tumor sites
3) Filter out all sSNVs in chromosome regions with LOH in all specimens from one tumor site but not in all samples from the other tumor site.
4) For sSNVs not present in any specimens with LOH, filter out sSNVs satisfying the following criteria in specimens from at least one of the two tumor sites: a) absent in some samples with LOH; b) not absent in any samples without LOH.

### Driver Enrichment Analysis

Driver fold enrichment was determined based on colorectal adenocarcinoma (COAD) driver genes (defined by combining IntOGen v.2016.5 and TCGA including 221 genes, Table 2) or all pan-cancer drivers, including 369 high-confidence genes harboring non-silent coding sSNVs out of the total number of genes with non-silent coding sSNVs. The resulting metric was normalized by the fraction of driver genes out of all genes in the human genome. Clonal mutations (CCF>60% in P or M; merged CCF was used for MRS data) were divided into three sets representing shared, primary-private and metastasis-private mutations, where only distant metastases were considered. Driver gene fold enrichment was calculated for each set of mutations by randomly sampling 15 of 25 P/M pairs from the whole cohort, aggregating them to calculate one driver enrichment score, and repeating this 100 times (n=100 down-samplings) to derive a test statistic. For each down-sampling, the driver enrichment score was calculated as:

$$Enrichment fold score = \frac{n ( driver \text{non-silent} clonal ) / n ( all \text{non-silent} clonal )}{n ( driver genes ) / n ( total genes )} \qquad EQ . 2$$

where n(all non-silent clonal) and n(driver non-silent clonal) correspond to the total number of non-silent clonal mutations and the number of non-silent clonal mutations in driver genes, respectively. Here n(driver genes) and n(total genes) correspond to the total number of drivers reported for CRC (n=221) or pan-cancer (n=369) and the number of coding genes in the genome (n=22,000), respectively.

### Prediction of Driver Gene Pathogenicity and Functional Impact

Beyond the focus on non-silent alterations (including non-silent SSNVs/indels including missense, stop gain and splicing SSNVs and indels), one can evaluate the predicted pathogenicity or functional impact (“driverness”) of mutations via numerous computational algorithms such as VEP (https://uswest.ensembl.org/Tools/VEP), FATHMM (http://fathmm.biocompute.org.uk/cancer.html) and CADD (https://cadd.gs.washington.edu/). For VEP, a SSNV/indel is considered as “functional” when the functional impact assessment is “HIGH” or “MODERATE”. For FATHMM, a SSNV is considered as “functional” if the “fathmm_score” is smaller than −0.75 (a default prediction threshold). For CADD (v1.4), a SSNV is considered as “functional” when the “CADD_PHRED” score is larger than 10 (a default prediction threshold). These methods can be used to further prioritize or rank the functional impact of specific mutations in the metastasis associated driver gene modules.

### Orthogonal Validation of Early Metastasis Driver Gene Modules

The MSK-Impact cohort includes early-stage primary CRCs, primary CRCs that are known to have metastasized and the metastatic lesion (predominantly liver) from 1,099 mCRC patients and a total of 1,134 samples with available sequencing and clinical covariates including stage, microsatellite status, and time to metastasis. Since the mCRC “discovery” cohort did not include microsatellite unstable (MSI+) cases, these were removed as were cases with POLE mutations. Microsatellite stable (MSS) samples were divided into early-stage non-metastatic samples (n=57), metastatic primary tumors (n=440) and metastatic samples (n=498).

The GENIE cohort is composed of 39,600 samples profiled with different targeted sequencing panels from which CRC samples were selected (oncotree codes: COADREAD, COAD, CAIS, MACR, READ and SRCCR). In order to avoid duplicated samples, all MSK-Impact samples from the GENIE cohort were removed, as were duplicated samples from the same patient, resulting in 2,666 samples, 1,756 of which were from primary tumors. As the GENIE cohort does not currently include stage or outcome information, all primaries are assumed to be non-metastatic, although some may be stage IV or diagnosed as metastatic in the future.

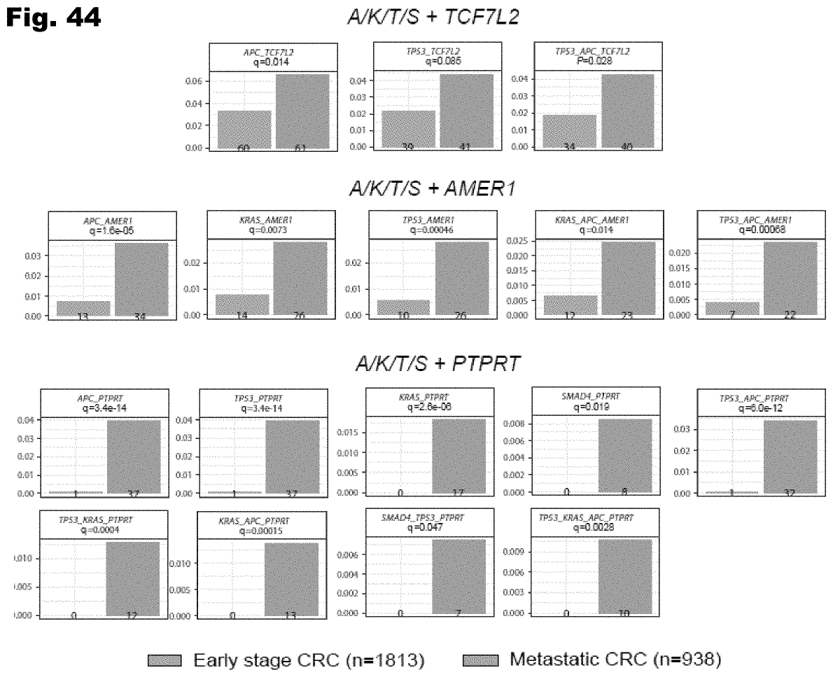

All possible combinations of recurrent putative M-driver genes (APC, TP53, KRAS, SMAD4, PIK3R1, BRAF, AMER1, TCF7L2, PIK3CA, PTPRT and ATM) identified in the mCRC cohort were evaluated in metastatic relative to early stage cases using a two-sided Fisher's exact test (Benjamini-Hochberg adjustment for multiple testing). The enrichment analysis was calculated for the combined MSK-Impact and GENIE primary CRC cohort, as well as for the MSK-Impact cohort alone. Importantly, as the number of genes in a module increases, the specificity of the association with metastasis increases, but the frequency of the module and in turn power to detect an association decreases (FIG. 44). While combining datasets may potentially introduce some biases, because it was assumed that all GENIE primary samples are non-metastatic and MSS, this will render the analyses conservative. Indeed, it is worth noting that while these results are already highly significant, they are likely conservative for several reasons: i) due to the short follow-up time, some early-stage cases may develop metastases, ii) imbalanced sample size with nearly twice as many early stage versus metastatic cases, iii) several putative M-drivers identified in the mCRC cohort are not represented on the targeted sequencing panel and hence cannot be evaluated. Importantly, these analyses can be performed on other sequencing datasets as they become available, thus expanding the samples size and power to detect additional gene modules associated with metastasis. Some such sources of data include the commercially available Foundation One (Foundation Medicine) targeted sequencing assay for solid tumors and the MSK-Impact data available in GENIE, each of which includes a number of these genes.

### Phylogenetic Tree Reconstruction and F_{ST} Computation

PHYLIP (http://www.trex.uqam.ca/index.php?action=phylip&app=dnapars) was utilized and the Maximum Parsimony method was applied to reconstruct the phylogeny of multiple specimens from individual patients based on the presence or absence of SNVs and indels (for more on PHYLIP, see J. Felsenstein, Cladistics 5, 164-166 (1989), the disclosure of which is herein incorporated by reference). When multiple maximum parsimony trees were reported, the top ranked solution was chosen. FigTree (http://tree.bio.ed.ac.uk/software/Figtree/) was employed to visualize the reconstructed trees. The FST statistic was computed for each primary tumor or metastasis using the Weir and Cockerham method based on the adjusted frequency of subclonal sSNVs (merged CCF<60%) identified in MRS data. Clonal mutations (merged CCF>60%) don't contribute to ITH and were excluded in FST calculations (for more on Cockerham method, see B. S. Weir and C. C. Cockerham, Evolution 38, 1358-1370 (1984), the disclosure of which is herein incorporated by reference).

### Spatial Agent-Based Modeling of Tumor Progression

The previously described three-dimensional agent-based tumor evolution framework was extended to model tumor growth, mutation accumulation and metastatic dissemination after malignant transformation under different evolutionary scenarios in P/M pairs, namely Neutral/Neutral (N/N), Neutral/Selection (N/S), Selection/Neutral (S/N) or Selection/Selection (S/S) (framework previously described in A. Sottoriva, et al., Nat Genet 47, 209-16 (2015); and R. Sun. et al., Nat Genet 49, 1015-1024 (2017); the disclosures of which are each herein incorporated by reference). Pre-malignant clonal expansions prior to transformation do not alter the genetic heterogeneity within a tumor thus were not modeled and it was assumed that dissemination occurs after malignant transformation of the founding carcinoma cell since invasion (a cardinal feature of carcinomas) is a requirement for metastasis. This framework was previously employed to model primary tumor evolution (see R. Sun, et al., (2017), cited supra). In this model, spatial tumor growth is simulated via the expansion of deme subpopulations (composed of ˜5k cells with diploid genome), mimicking the glandular structures often found in colorectal tumors and metastases and consistent with the number of cells found in individual colorectal cancer glands (˜2,000-10,000 cells). Model assumptions are detailed in Table 3. The deme subpopulations expand within a defined 3D cubic lattice (Moore neighborhood, 26 neighbors), via peripheral growth while cells within each deme are well-mixed without spatial constraints and grow via a random birth-and-death process (division probability p and death probability q=1−p at each generation). The notion of peripheral growth is supported by recent studies indicating that cancer cells at the periphery of the tumor proliferate much faster than those at the center (see M. C. Lloyd, et al., Cancer Res 76, 3136-44 (2016), the disclosure of which is herein incorporated by reference). Moreover, peripheral growth results in a power law model of net tumor growth, and is supported by data in colorectal cancer (see E. A. Sarapata and L. G. de Pillis Bull Math Biol 76, 2010-24 (2014), the disclosure of which is herein incorporated by reference). The first deme is generated via the same birth-and-death process, beginning with a single transformed founding tumor cell. Here we employ the following parameters: p=0.55 and q=0.45 for the deme expansion in both the primary tumor and metastasis. Thus the cell birth/death probability ratio for the founding lineage is p/q=0.55/0.451.2. This is supported by the observation that there is no significant difference in proliferation rates based on Ki67 staining of paired CRCs and brain metastases (FIG. 21). Based on these values of p and q, approximately 3 years are required from transformation to the diagnosis of primary carcinoma (˜10⁹ cells) (FIG. 30). Once a deme exceeds the maximum size (10,000 cells), it splits into two offspring demes via random sampling of cells from a binomial distribution [N_{c}, p=0.5], where N_{c} is the current deme size.

During the growth of the primary CRC, a single cell from a random deme at the tumor periphery is randomly chosen to seed the metastasis supported by mounting pathological evidence of invasive cells in tumor front and that blood vessels are also mostly distributed in the invasive front in CRC. The total cell number at the time of metastatic dissemination is denoted by N_{d}. The metastasis grows via the same model as the primary tumor, starting from the disseminated tumor cell(s).

During each cell division, the number of neutral passenger mutations acquired in the coding portion of the genome follows a Poisson distribution with mean u. Thus, the probability that k mutations occurred in each cell division is as follows:

$$P ( x = k ) = \frac{{u}^{k} {e}^{- u}}{k !} \qquad EQ . 3$$

where an infinite sites model and constant mutation rate are assumed during tumor progression. For simplicity, CNAs, LOH, an aneuploidy were not simulated, and all mutations were considered heterozygous. Under the neutral model, all somatic mutations are assumed to be neutral passenger events and do not confer a fitness advantage, whereas in the subclonal selection model, beneficial mutations (or advantageous mutations) arise stochastically via a Poisson process with mean uₛ during each cell division. It was assumed uₛ=10⁻⁵ per cell division in the genome. A relatively strong positive selection coefficient (s=0.1) was further investigated, where s specifies the increase in cell division probability per cell division when a beneficial mutation occurs in the neutral cell lineage. The cell birth and death probabilities for a selectively beneficial clone are pₛ=p×(1+s) and qₛ=1−pₛ=1−p×(1+s), respectively, thus the selective advantage is defined as s=pₛ/p−1. s=0.1 was selected since it was previously shown that the resultant patterns of between-region genetic divergence can be clearly distinguished from those arising under effectively neutral growth (see R. Sun, et al., (2017), cited supra).

During simulation of primary and metastatic growth, each mutation is assigned a unique index that is recorded with respect to its genealogy and host cells, enabling analysis of the mutational frequency in a sample of tumor cells or the whole tumor during different stages of growth. Growth was simulated until the primary and metastasis reach a size of ˜10⁹ cells (or ˜10 cm³) comparable to the size of the clinical samples studied here which ranged from 4-15 cm in maximum diameter. To simulate each of the four scenarios of P/M growth, namely N/N, N/S, S/N or SS, a mutation rate u=0.3 per cell division was employed in the exonic region (corresponding to 5×10⁻⁹ per site per cell division in the 60 Mb diploid coding regions) and selection coefficients s=0 and s=0.1 were employed when modeling neutral evolution and subclonal selection, respectively, during growth of the primary tumor or metastasis. Under each of the four scenarios of P/M growth, 100 time points (representing the primary tumor size at the time of dissemination, N_{d}) were sampled at random from a uniform distribution, log10(N_{d})˜U(2,9), each giving rise to independent P/M pairs. The CCF from the whole tumor in both the P and M lesions were obtained for each sSNV (site). CCFs>60% in one site and CCFs<1% in the other site were used to count the number of P-private and M-private clonal sSNVs (Lₚ and Lₘ, respectively), consistent with the strategy employed for patient samples.

### Spatial Computational Inference of MEtastatic Timing (SCIMET)

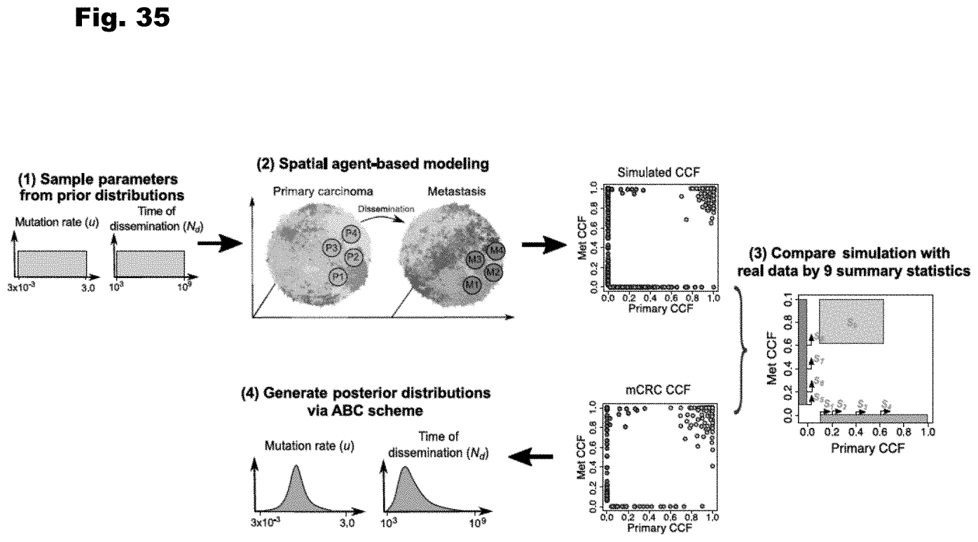

It was sought to infer two parameters that govern the dynamics of metastasis, namely u, the mutation rate per cell division in the exonic region and N_{d}, primary tumor size at the time of dissemination based on our spatial tumor simulation framework. The two parameters of interest (u and N_{d}) were randomly sampled from a prior discrete uniform distribution, namely 10 values from 0.003 to 3 for u; and 7 values from 10³ to 10⁹ cells (on log10 scale) for N_{d} (FIG. 35, Tables 4 and 5). Discrete prior distributions for u and N_{d} were used to estimate the order of magnitude rather the precise values of these two parameters. 70,000 paired primary tumors and metastases (composed of 10⁹ cells each) were simulated under each of the four evolutionary scenarios (N/N, N/S, S/N or S/S). After generating the virtual P/M tumors, multiple regions (n=4) each composed of ˜10⁶ cells are sampled from an octant of tumor sphere, as was done for the clinical samples (FIG. 35). The VAF of all sSNVs in the sampled bulk subpopulation is considered the true VAF (denoted by f_{T}), whereas the observed allele frequency is obtained via a statistical model that mimics the random sampling of alleles during sequencing. Specifically, a Binomial distribution (n, f_{T}) was employed to generate the observed VAF at each site given its true frequency f_{T} and number of covered reads n. The number of covered reads at each site is assumed to follow a negative-binomial distribution (negative binomial(size, depth)) where depth is the mean sequencing depth and size corresponds to the variation parameter. It was assumed depth=80 and size=2 for the sequencing data in each tumor region. A mutation is called when the number of variant reads is thereby applying the same criteria as for the patient tumors. The observed VAF for each mutation is converted to CCF and the merged CCF from four regions were computed (Eq.(1)) to mimic the patient genomic data. The nine summary statistics used to fit the CCF data are described in FIG. 35 and Table 4 The median values of the posterior probability distributions obtained from SCIMET are referred to as the inferred parameter values (u and Ñ_{d}). To be conservative, we define early dissemination as N_{d} (upper bound)<10⁸ cells (˜1 cm³ in volume) using the 3^{rd} quartile of the posterior distribution as the upper bound, whereas late dissemination is defined as N_{d} (upper bound)≥10⁸ cells (FIG. 37). The robustness of SCIMET to a higher birth/death rate ratio (FIG. 38), collective dissemination by a cell cluster (n=10 cells, FIG. 39) or single-region sequencing data (FIG. 40) were also evaluated. Of note, both a higher birth/death rate ratio and single-region sequencing data would result in overestimation of the timing of metastatic dissemination. A higher birth/death rate ratio yields a higher tumor growth rate thus the primary tumor size at the time of dissemination would be larger than for a lower birth/death rate ratio. Single-region sampling results in a larger number of metastasis-private clonal mutations (larger Lₘ and larger H) compared with multi-region sequencing, thus the timing of dissemination would be overestimated in accordance with the positive correlation between Lₘ or H and N_{d}. Overall, these comparisons demonstrate the robustness of SCIMET to different model assumptions.

A version of ABC based on the Acceptance-Rejection Algorithm was employ to estimate posterior probability distributions for the parameters of interest θ(u, N_{d}) (for more on Acceptance-Rejection Algorithm, see S. Tavare, et al., Genetics 145, 505-18 (1997), the disclosure of which is herein incorporated by reference). The ABC version of rejection sampling is as follows:

For i=1 to K under model M(N/N, N/S, S/N or S/S):
1. Sample parameters θ′ from the prior distribution π(θ)
2. Simulate data D′ using model M with the sampled parameters θ′, and summarize D′ as summary statistics S′
3. Accept θ′ if d(S′, S)<ϵ, for a given tolerance rate ϵ, where d(S′, S) is a measure of Euclidean distance between S′ and S
4. Go to 1

This scheme was able to approximate the posterior distribution by: P(θ|d(S′, S)<ϵ). a common variation of ABC was used where rather than using a fixed threshold, ϵ, all K distances were sorted and calculated in by d(S′, S) (Step 3), and accepted the θ′ that generated the smallest 100×η percent distances. η=0.01 was used so that the posterior is composed of 70,000×0.01=700 data points (for more on the common variation of ABC, see M. A. Beaumont, W. Zhang, and D. J. Balding, Genetics 162, 2025-35 (2002); and J. Zhao, et al., J Theor Biol 359, 136-45 (2014), the disclosures of which are each herein incorporated by reference). The ABC procedure is performed using the R package abc (see K. Csillery, O. Francois, and M. G. Blum, Methods in ecology and evolution 3, 475-479 (2012), the disclosure of which is herein incorporated by reference). To determine the most probable model of tumor evolution (N/N, N/S, S/N or S/S) in P/M pairs, the postpr method implemented in the R package abc was ran, which integrates all simulation data from the four models to run the ABC procedures (steps 1-4) and outputs the probability of each model based on the posterior distribution. The model (N/N, N/S, S/N or S/S) with the highest probability was selected.

A Monte Carlo cross-validation scheme was performed to assess the performance of SCIMET. This procedure involves randomly sampling a combination of parameters u′ and N_{d}′ (true parameters) and sampling 10 simulations of the summary statistics S′ under this parameter set to independently run the ABC scheme. The posterior parameters u and N_{d} with the maximum probability were used as parameter estimates for one simulation. The mean value of posterior u′s and N_{d}′s in 10 simulations was taken as the parameter estimate (inferred parameters). The process of Monte Carlo sampling and SCIMET inference was repeated 200 times under each of the four evolutionary scenarios (N/N, N/S, S/N, and S/S). Comparison of the inferred versus true parameter values indicates the robustness of this approach (FIG. 36).

### Example 2

### Co-Occurrence of Gene Drivers in Colorectal Cancer

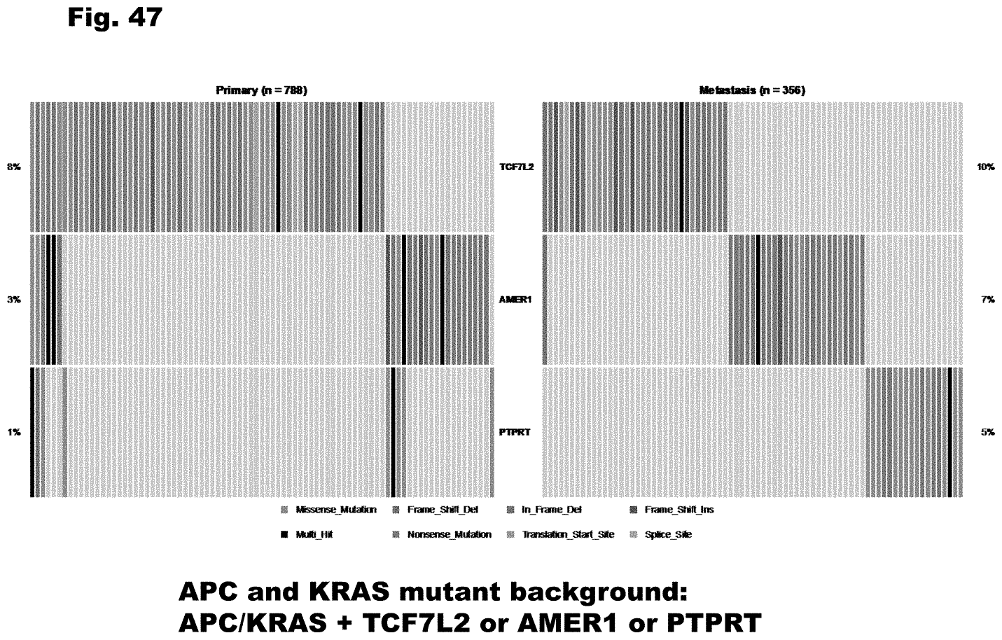

Based on the data results across a validation cohort, it is now appreciated that the genetic aberrations in PTPRT, TCF7L2, and AMER1 co-occur with APC, KRAS, TP53, and/or SMAD4 to drive a colorectal cancer into an aggressive phenotype and high potential for metastasis. Provided in FIGS. 47 to 51 are co-occurrence plots demonstrating the results of at least one of: PTPRT, TCF7L2, and AMER1 to have a genetic aberration and co-occurring with a number of combinations of APC, KRAS, TP53, and/or SMAD4 having a genetic aberration. Each figure depicts a number of patients (each column is a patient) having a particular genetic aberration (denoted by color) in one of the genes PTPRT, TCF7L2, and AMER1 (each row is one gene). On the left are patients that only experienced a primary tumor (and no metastasis as of time of the data collected). On the right are patients that experienced a metastatic event. Each figure is filtered to a subset of patients having genetic aberrations in a combination of A/K/T/S.

In FIG. 47, the co-occurrence plot depicts patients having genetic aberrations in both APC and KRAS co-occurring with genetic aberrations in at least one of: PTPRT, TCF7L2, and AMER1. As can be seen, a high percentage of patients having genetic aberrations in both APC and KRAS co-occurring with genetic aberrations in at least one of: PTPRT, TCF7L2, and AMER1 also experienced a metastatic event (22%), whereas only 12% of patients only experienced a primary tumor.

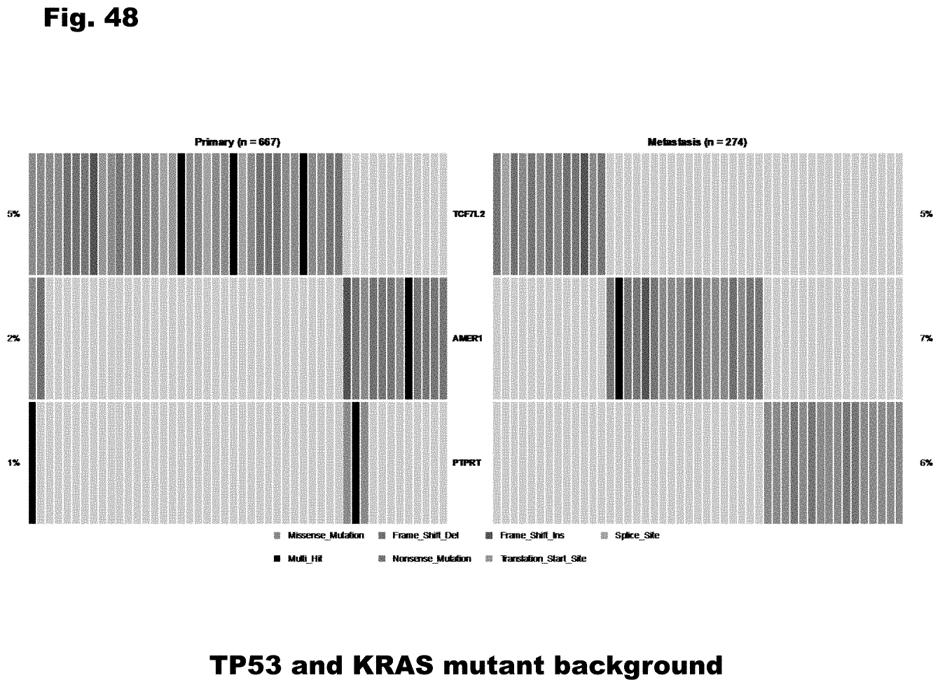

In FIG. 48, the co-occurrence plot depicts patients having genetic aberrations in both TP53 and KRAS co-occurring with genetic aberrations in at least one of: PTPRT, TCF7L2, and AMER1. As can be seen, a high percentage of patients having genetic aberrations in both TP53 and KRAS co-occurring with genetic aberrations in at least one of: PTPRT, TCF7L2, and AMER1 also experienced a metastatic event (18%), whereas only 8% of patients only experienced a primary tumor.

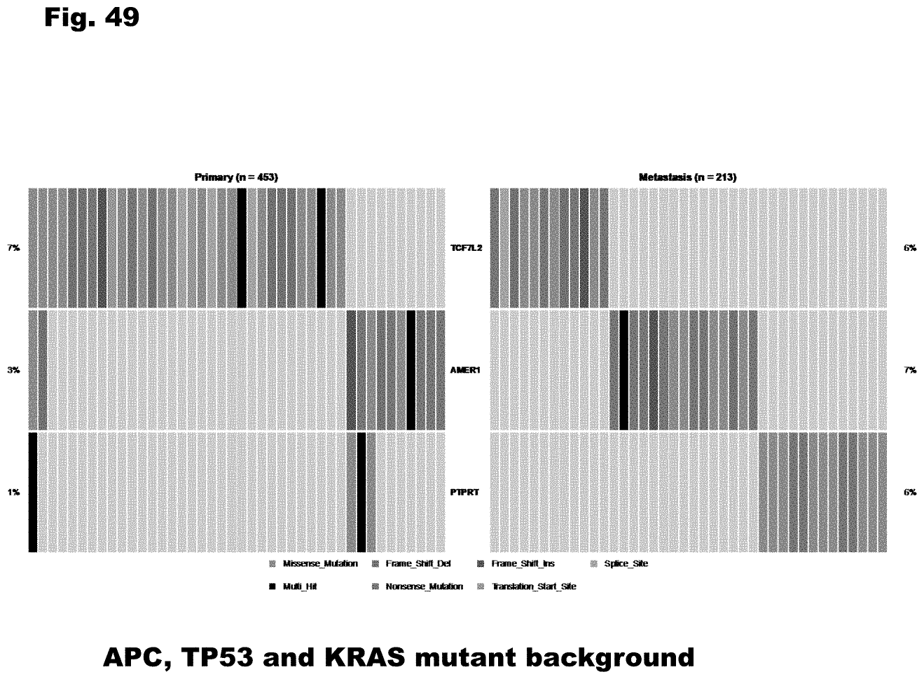

In FIG. 49, the co-occurrence plot depicts patients having genetic aberrations in both APC, TP53 and KRAS co-occurring with genetic aberrations in at least one of: PTPRT, TCF7L2, and AMER1. As can be seen, a high percentage of patients having genetic aberrations in both APC, TP53 and KRAS co-occurring with genetic aberrations in at least one of: PTPRT, TCF7L2, and AMER1 also experienced a metastatic event (19%), whereas only 11° A of patients only experienced a primary tumor.

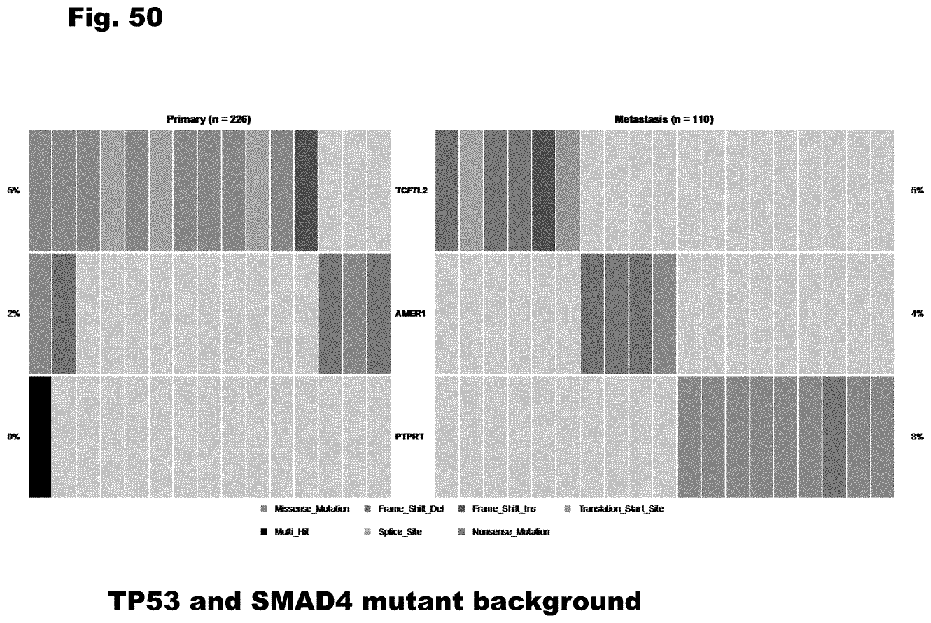

In FIG. 50, the co-occurrence plot depicts patients having genetic aberrations in both TP53 and SMAD4 co-occurring with genetic aberrations in at least one of: PTPRT, TCF7L2, and AMER1. As can be seen, a high percentage of patients having genetic aberrations in both TP53 and SMAD4 co-occurring with genetic aberrations in at least one of: PTPRT, TCF7L2, and AMER1 also experienced a metastatic event (17%), whereas only 7% of patients only experienced a primary tumor.

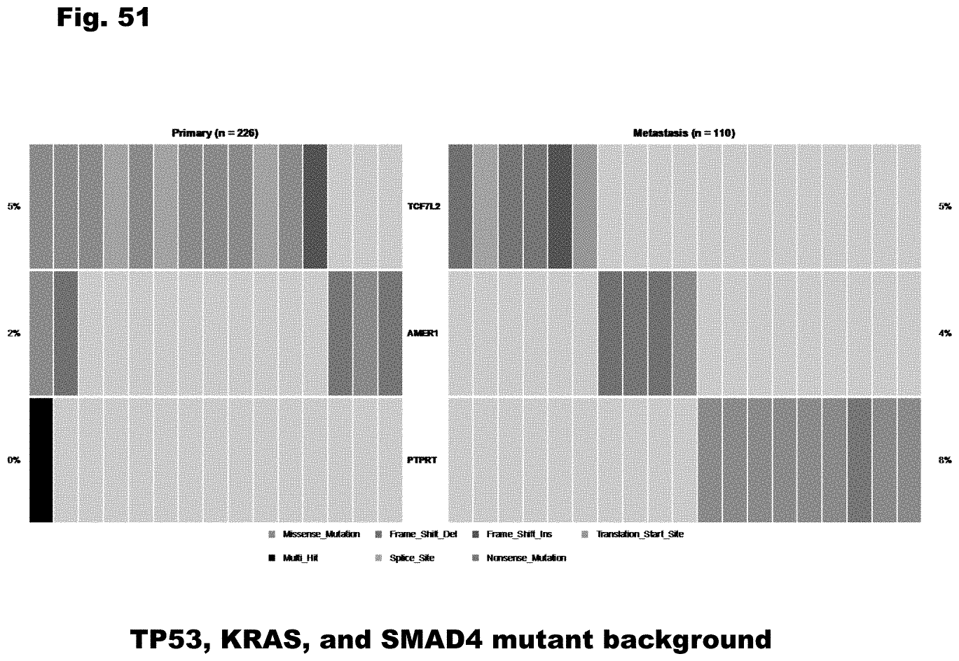

In FIG. 51, the co-occurrence plot depicts patients having genetic aberrations in both TP53, KRAS and SMAD4 co-occurring with genetic aberrations in at least one of: PTPRT, TCF7L2, and AMER1. As can be seen, a high percentage of patients having genetic aberrations in both TP53, KRAS and SMAD4 co-occurring with genetic aberrations in at least one of: PTPRT, TCF7L2, and AMER1 also experienced a metastatic event (17%), whereas only 7% of patients only experienced a primary tumor.

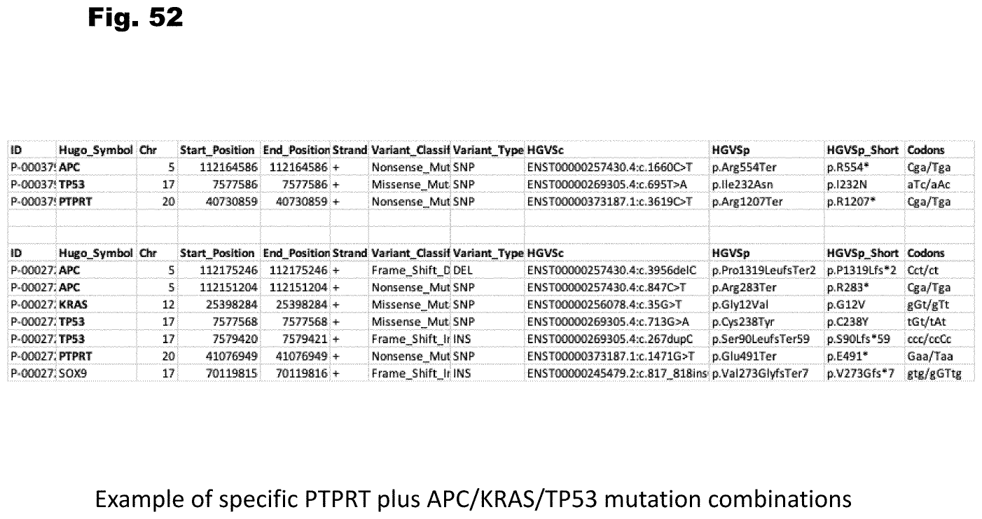

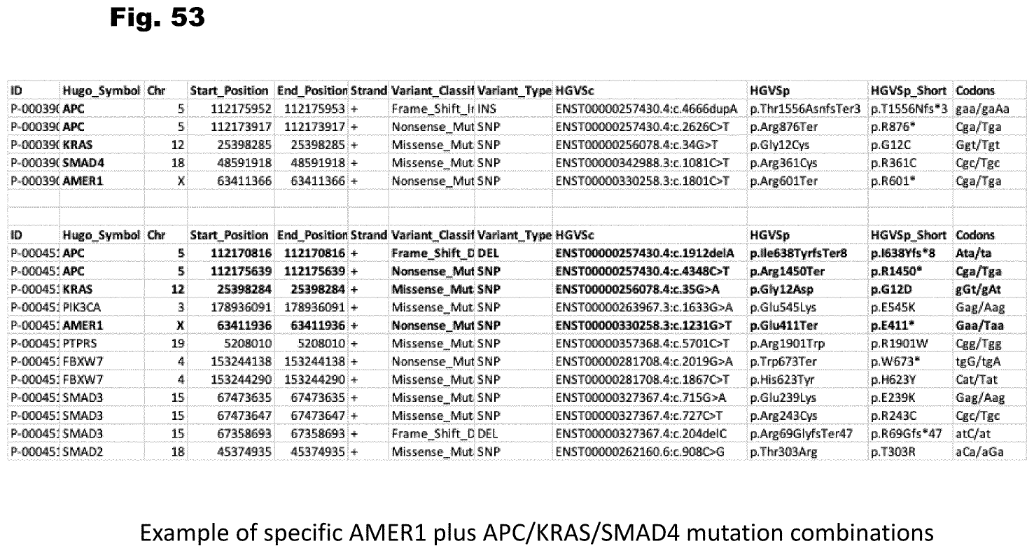

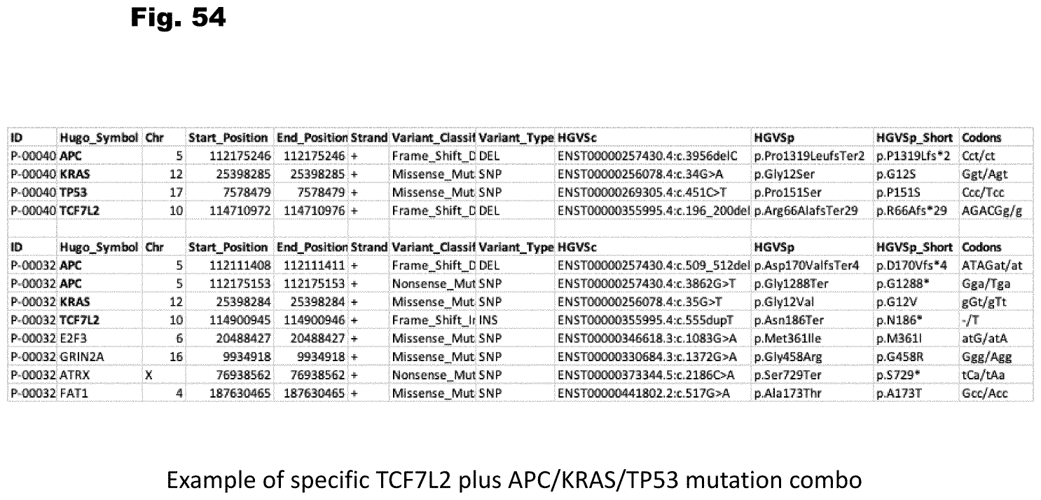

Provided in FIGS. 52 to 55 are tables displaying exemplary colorectal patients that each experienced a metastatic event. Within the tables are each patient's genetic aberrations that were discovered, each having genetic aberration of one of PTPRT, TCF7L2, and AMER1 co-occurring with APC, KRAS, TP53, and/or SMAD4. Within each figure are two patients. In FIG. 52, each patient has genetic aberrations in the combination of genes PTPRT with APC, KRAS and TP53. In FIG. 53, each patient has genetic aberrations in the combination of genes AMER1 with APC, KRAS and SMAD4. In FIG. 54, each patient has genetic aberrations in the combination of genes TCF7L2 with APC, KRAS and TP53. In FIG. 55, each patient has genetic aberrations in the combination of genes TCF7L2 with APC and KRAS.

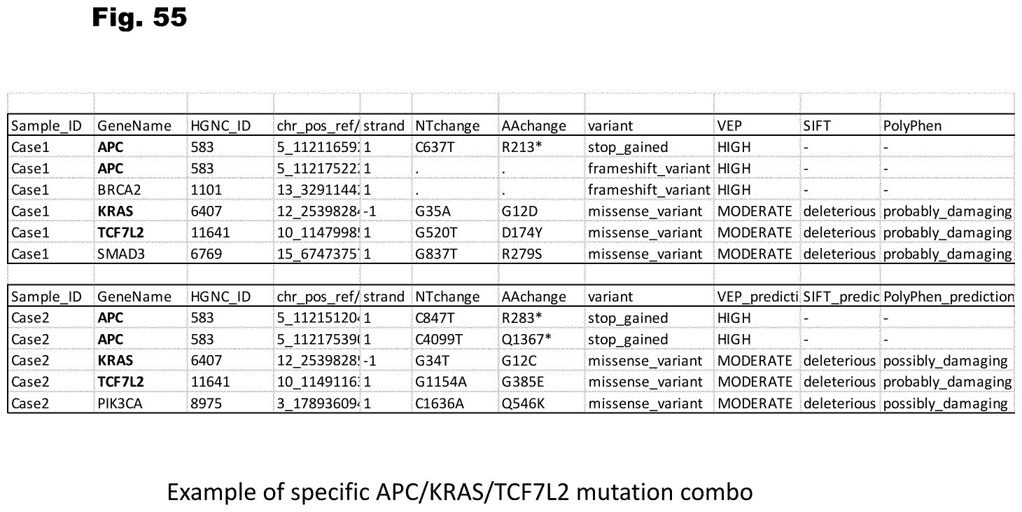

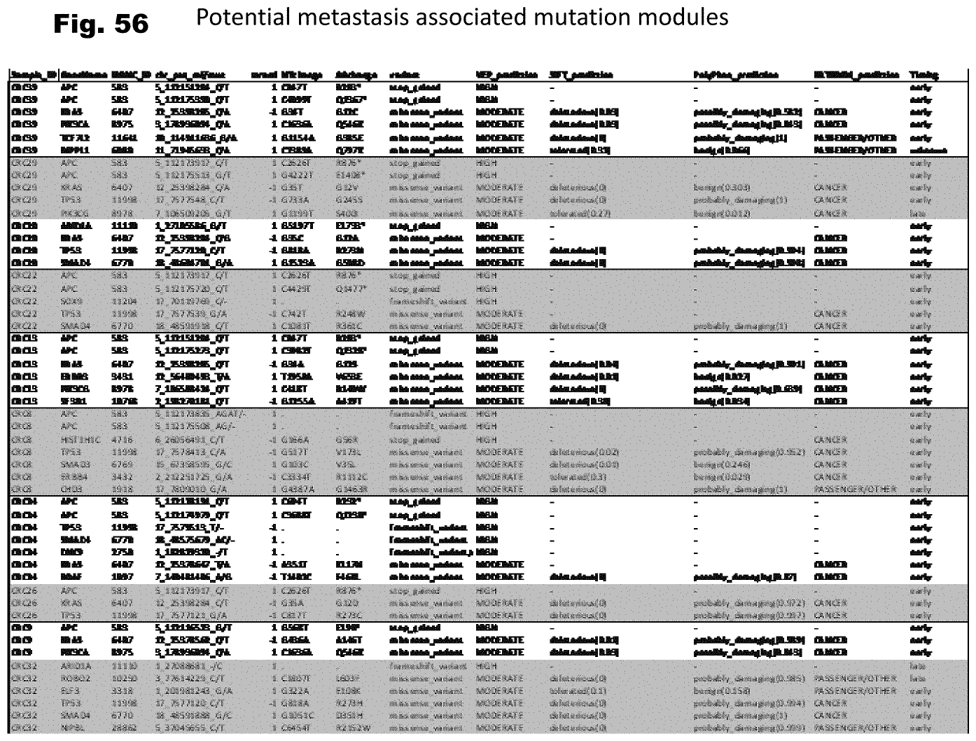

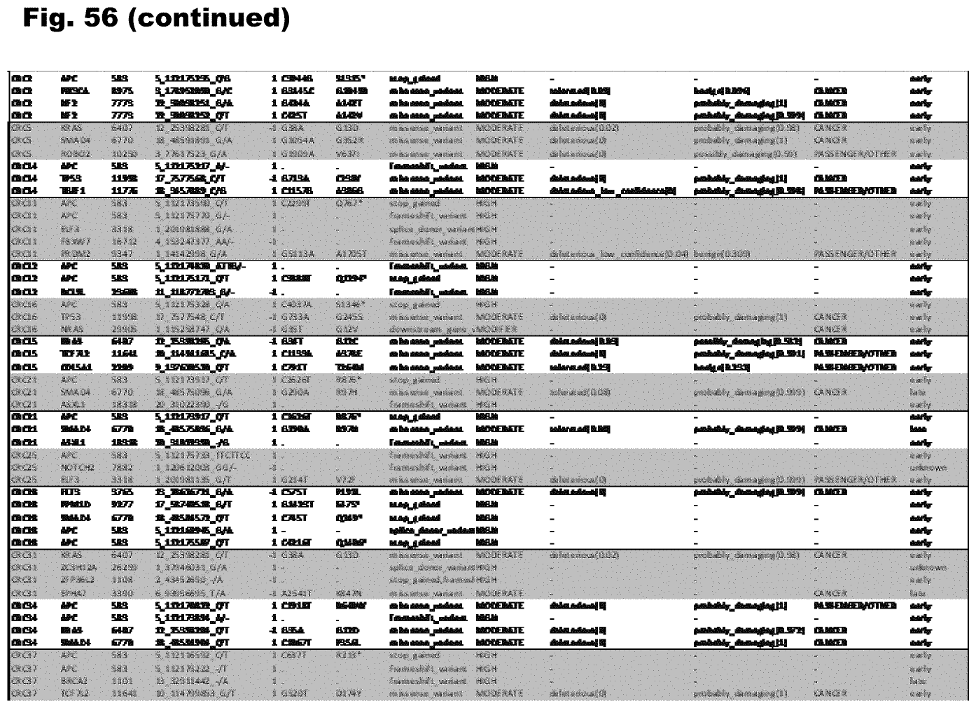

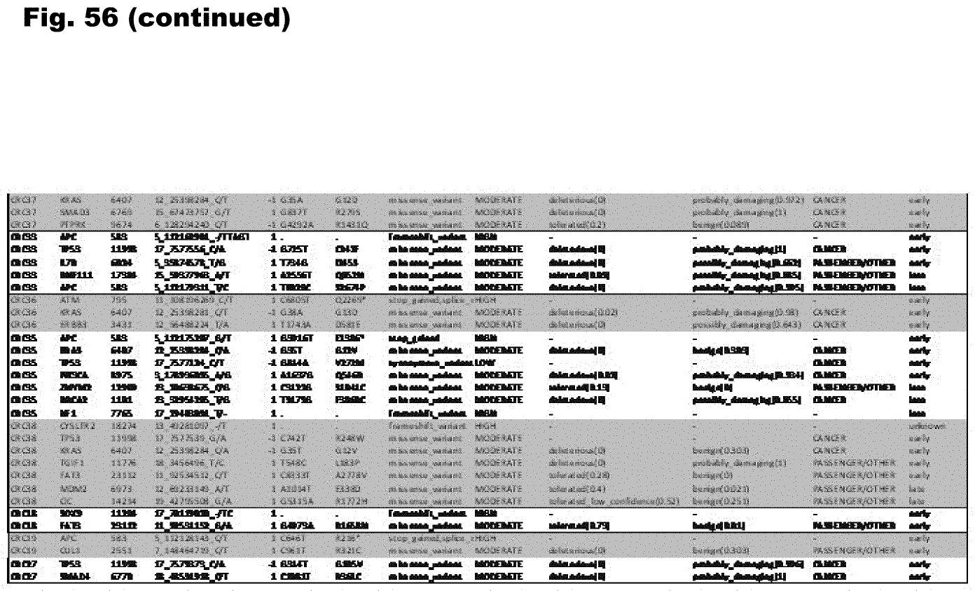

Provided in FIG. 56 is a table with potential gene combinatorial that may confer aggressiveness and metastatic potential when each gene harbors a genetic aberration. The combinatorial set of genes are shown in the second column and divided in rows by shading. For example, in the first row, sample CR C39 had genetic aberrations in the combinatorial set of genes of APC, KRAS, PIK3CA, TCF7L2, and INPPL1.

### Example 3

### Genetic Aberrations that Confer Oncogenic Potential

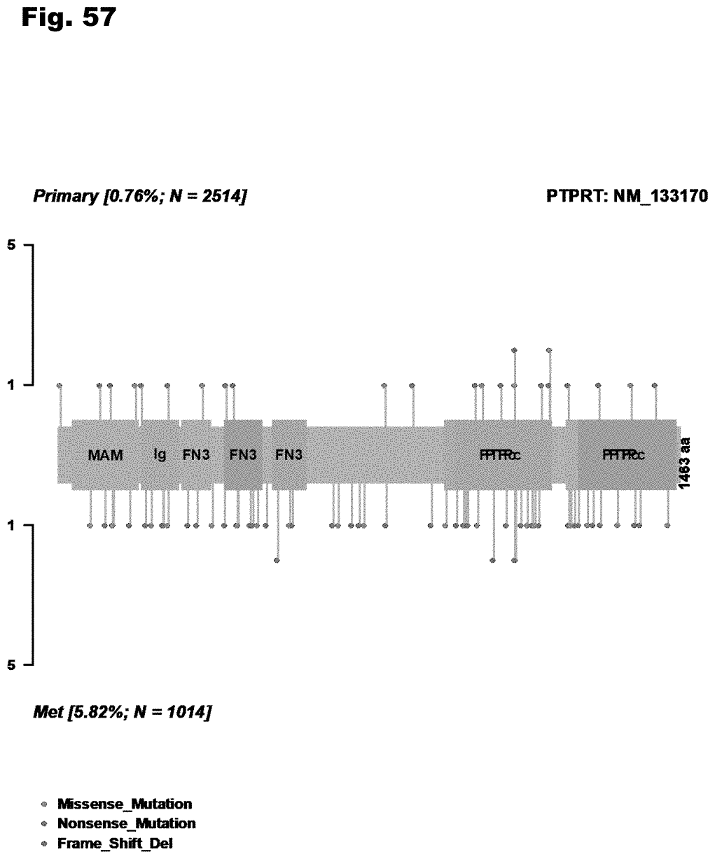

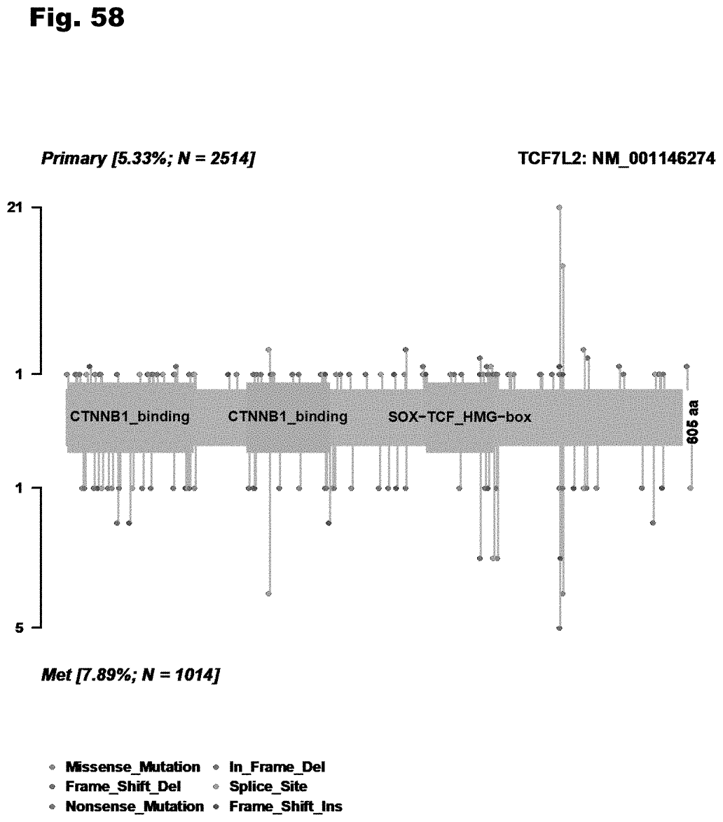

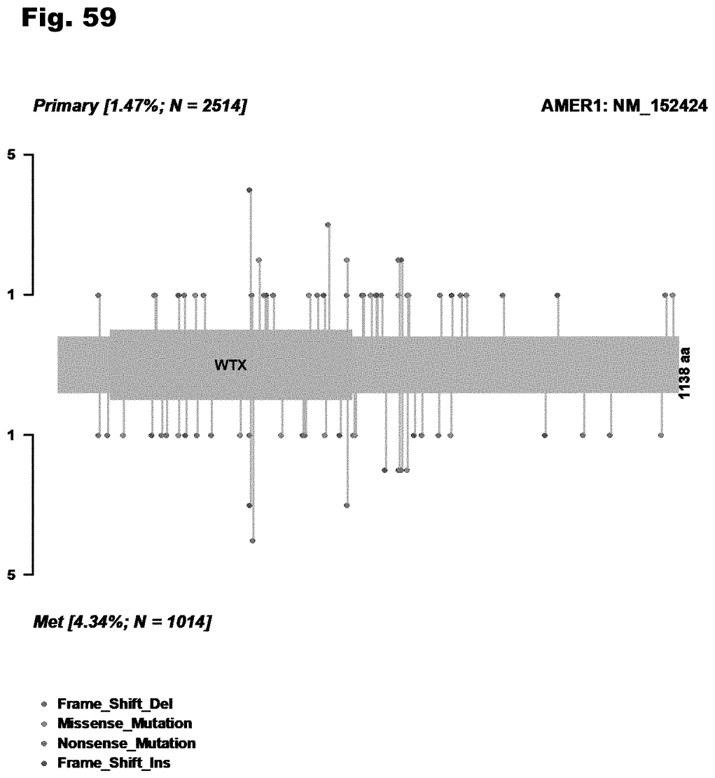

Provided in FIGS. 57 to 59 are lollipop plots that show a number of known genetic aberrations that occur in PTPRT, TCF7L2, and AMER1 in various cancers. These genetic aberrations can provide diagnostic information in regards to PTPRT, TCF7L2, and AMER1. It is noted however, that many genetic aberrations not depicted may also provide an oncogenic effect and result in high aggression and metastatic potential.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

TABLE 1
Clinical features of the metastatic colorectal cancer cohort

| | | Age at | | Treatment | | | | Number | | | |
|---|---|---|---|---|---|---|---|---|---|---|---|
| | | diagnosis | | before | Primary | Met | Total | of | | MRS | |
| | | of | | diagnosis of | tumor | tumor | number | primary | | of both | |
| Patient | | primary | | asynchronous | size | size | of | tumor | Number of | Primary | |
| ID | Sex | tumor | Diagnosis history | met | (cm) | (cm) | samples | samples | met samples | and Met | Data source |
| V46 | M | 60 | P&LN-LU(2 y 7 m)- | yes | 4 | 3 (BM) | 3 | 1 | 1 (BM), 1(LN) | no | This study |
| | | | BM(3 y 9 m) | | | | | | | | |
| V402 | F | 47 | P-BM(4 y 8 m) | no | 7.5 | 5 (BM) | 8 | 4 | 4 (BM) | yes | This study |
| V514 | M | 73 | P&LN-BM(0 y 6 m) | yes | 9 | 2.5 (BM) | 5 | 1 | 2 (BM), 2 (LN) | no | This study |
| V559 | M | 49 | P&LI-LU(1 y 5 m)- | yes | 4.5 | 3.5 (BM) | 4 | 1 | 1 (BM), 2 (LI) | no | This study |
| | | | BM(1 y 8 m) | | | | | | | | |
| V750 | M | 65 | P&LN&LI&LU- | yes | 10 | 3 (BM) | 13 | 5 | 5 (BM), 3 (LN) | yes | This study |
| | | | BM(0 y 6 m) | | | | | | | | |
| V824 | M | 61 | P&LN- | no | 8 | 5 (BM) | 9 | 3 | 3 (BM), 3 (LN) | yes | This study |
| | | | BM&LU(0 y 10 m) | | | | | | | | |
| V855 | M | 57 | P&LN-BM(0 y 4 m) | yes | 6 | 3 (BM) | 2 | 1 | 1 (BM) | no | This study |
| V930 | F | 71 | P-LI(2 y 2 m)- | yes | 4 | NA | 13 | 5 | 5 (BM), 3 (LU) | yes | This study |
| | | | LU(5 y 8 m)- | | | | | | | | |
| | | | BM(8 y 7 m) | | | | | | | | |
| V953 | F | 68 | P-BM(2 y 6 m) | no | 8.1 | 5 (BM) | 7 | 3 | 4 (BM) | yes | This study |
| V974 | F | 60 | P&BM- | no | 10 | 5 (BM) | 8 | 3 | 5 (BM) | yes | This study |
| | | | RecBM(0 y 5 m) | | | | | | | | |
| Uchi2 | M | 81 | P&LI | no | 5.2 | NA | 12 | 9 | 3 (LI) | yes | Uchi et al. 2016 |
| Kim1 | M | 69 | P&LI | no | 6 | NA | 7 | 4 | 3 (LI) | yes | Kim et al. 2015 |
| Kim2 | M | 79 | P-LI(0 y 7 m) | no | 10.5 | NA | 7 | 5 | 2 (LI) | yes | Kim et al. 2015 |
| Leung1 | M | 77 | P&LI | no | NA | NA | 2 | 1 | 1 (LI) | no | Leung |
| | | | | | | | | | | | et al. 2017 |
| Leung2 | M | 64 | P&LI | no | NA | NA | 2 | 1 | 1 (LI) | no | Leung |
| | | | | | | | | | | | et al. 2017 |
| Lim3 | M | 46 | P&LI | no | NA | NA | 2 | 1 | 1 (LI) | no | Lim et al. 2015 |
| Lim6 | M | 59 | P&LI | no | NA | NA | 2 | 1 | 1 (LI) | no | Lim et al. 2015 |
| Lim7 | F | 54 | P&LI | no | NA | NA | 2 | 1 | 1 (LI) | no | Lim et al. 2015 |
| Lim8 | M | 57 | P&LI | no | NA | NA | 2 | 1 | 1 (LI) | no | Lim et al. 2015 |
| Lim11 | M | 57 | P&LI | no | NA | NA | 2 | 1 | 1 (LI) | no | Lim et al. 2015 |
| Lim12 | M | 71 | P&LI | no | NA | NA | 2 | 1 | 1 (LI) | no | Lim et al. 2015 |
| Lim16 | M | 77 | P&LI | no | NA | NA | 2 | 1 | 1 (LI) | no | Lim et al. 2015 |
| Lim21 | M | 52 | P&LI | no | NA | NA | 2 | 1 | 1 (LI) | no | Lim et al. 2015 |
| Total | | | | | | | 118 | 55 | 63 | | |

Abbreviations: P—primary tumor; BM—brain metastasis; LN—lymph node metastasis; LI—liver metastasis; LU—lung metastasis; met—metastasis; MRS—multi-region sequencing; NA—not available; &—synchronous; m—month; y—year
Note:
samples from primary tumor and synchronously diagnosed metastases were untreated

TABLE 2
Driver Genes
TCGA lntogen CRC Driver Genes

| | | | | |
|---|---|---|---|---|
| CDH1 | SPDYE1 | ART5 | BMPR1A | PPP2R1A |
| APC | STARD3NL | MCHR2 | MYC | CNOT1 |
| TP53 | OR11G2 | SERPINB10 | IGF2 | CHD9 |
| KRAS | TWISTNB | OR4D10 | CCND2 | BRWD1 |
| FBXW7 | HIAT1 | PDCD5 | PREX2 | CEP290 |
| PIK3CA | KRTAP10-7 | HLA-DRA | FGF6 | TRIO |
| SMAD4 | CD3G | NIPA2 | FGF23 | MSR1 |
| AMER1 | NOL7 | THAP5 | ERBB2 | GOLGA5 |
| BRAF | ELAVL3 | TRMT10C | NUTM2B | TP53BP1 |
| NRAS | SLC3A2 | GPR141 | PARK2 | SMC1A |
| TCF7L2 | OR5K4 | IDH1 | FAM123B | NTN4 |
| CTNNB1 | INO80E | BCHE | ARID1A | MECOM |
| ADAM29 | CDC42EP1 | ROB02 | ATM | NUP98 |
| RNF43 | RPL22 | KRTAP10-6 | CHD4 | FOXP1 |
| EGFR | RNF145 | RIC8A | GNAS | RTN4 |
| RP1L1 | TNFAIP6 | COL6A5 | ELF3 | LPHN2 |
| ACVR2A | DDHD1 | AKAP7 | AXIN2 | ACO1 |
| ARHGAP5 | WDR55 | BCAP29 | RBM10 | MYH10 |
| PIK3R1 | SLC35G2 | ZNF233 | PTEN | ARNTL |
| FANCD2 | ESRP1 | OR7C1 | DNMT3A | CDC73 |
| CEP164 | ZNF365 | HNRNPL | STAG2 | TAF1 |
| FMN2 | ORAI1 | OR2M3 | NF1 | CDK12 |
| TSHR | BRD3 | TCERG1 | MLL2 | BPTF |
| JAK2 | TXNDC2 | CCDC28A | WT1 | MGA |
| PCBP1 | LMAN1 | RBM43 | CASP8 | BCOR |
| PPP1R12B | KRT4 | ATXN3L | CTCF | NR2F2 |
| CLOCK | SOX9 | PROSER3 | IDH2 | SRGAP3 |
| KIT | IVL | OR2M2 | ATRX | ITSN1 |
| DKK2 | EYS | MYOM1 | GATA3 | POLR2B |
| MUC2 | MUC4 | GPR174 | CDKN1B | CUL1 |
| ZNF516 | HLA-B | HTR2A | MAP2K1 | ZC3H11A |
| RHPN2 | B2M | OR5V1 | FN1 | PTGS1 |
| CASP5 | CYLC1 | BCL9L | PTPRU | NUP107 |
| TGIF1 | KRTAP4-3 | C6ORF89 | CAD | FXR1 |
| RLIM | POLI | MUC15 | TGFBR2 | SF3B1 |
| LIG1 | OR6C75 | ALDH3B1 | AKAP9 | WIPF1 |
| OR6C76 | PRKRA | ARSJ | CREBBP | TBX3 |
| CBL | KRTAP5-5 | KCTD16 | DIS3 | SYNCRIP |
| TEAD2 | ACVR1B | RHOA | MED12 | TCF12 |
| RUNX1 | SNX13 | ALDH2 | MED24 | NR4A2 |
| NEFH | LURAP1L | P2RY13 | ASPM | ACSL6 |
| TMBIM4 | SMAD2 | GPRIN1 | MAP3K4 | RAD21 |
| ESRRA | KIF25 | SGK223 | CLSPN | PTPN11 |
| SLAMF1 | SELPLG | TBC1D23 | SOS2 | BMPR2 |
| ABCF1 | | | | |

Pan-cancer Driver Genes

| | | | | |
|---|---|---|---|---|
| ABL1 | CNOT3 | HIST1H1C | NIPBL | SLC4A5 |
| ACO1 | COL2A1 | HIST1H1E | NOTCH1 | SMAD2 |
| ACVR1 | COL5A1 | HIST1H2BD | NOTCH2 | SMAD4 |
| ACVR1B | COL5A3 | HIST1H3B | NPM1 | SMARCA4 |
| ACVR2A | CREBBP | HIST1H4E | NRAS | SMARCB1 |
| ACVR2B | CRLF2 | HLA-A | NSD1 | SMC1A |
| ADNP | CSDE1 | HLA-B | NT5C2 | SMC3 |
| AJUBA | CSF1R | HLA-C | NTN4 | SMO |
| AKT1 | CSF3R | HNF1A | NTRK3 | SMTNL2 |
| ALB | CTCF | HOXB3 | NUP210L | SNX25 |
| ALK | CTNNA1 | HRAS | OMA1 | SOCS1 |
| ALPK2 | CTNNB1 | IDH1 | OR4A16 | SOX17 |
| AMER1 | CUL3 | IDH2 | OR4N2 | SOX9 |
| APC | CUL4B | IKBKB | OR52N1 | SPEN |
| APOL2 | CUX1 | IKZF1 | OTUD7A | SPOP |
| ARHGAP35 | CYLD | ILEST | PAPD5 | SPTAN1 |
| ARHGAP5 | DAXX | IL7R | PAX5 | SRC |
| ARID1A | DDX3X | ING1 | PBRM1 | SRSF2 |
| ARID1B | DDX5 | INTS12 | PCBP1 | STAG2 |
| ARID2 | DIAPH1 | IPO7 | PDAP1 | STAT3 |
| ARID5B | DICER1 | IRF4 | PDGFRA | STAT5B |
| ASXL1 | DIS3 | ITGB7 | PDSS2 | STK11 |
| ATM | DNM2 | ITPKB | PDYN | STK19 |
| ATP1A1 | DNMT3A | JAK1 | PHF6 | STX2 |
| ATP1B1 | EEF1A1 | JAK2 | PHOX2B | SUFU |
| ATP2B3 | EGFR | JAK3 | PIK3CA | TBC1D12 |
| ATRX | ElF1AX | KANSL1 | PIK3R1 | TBL1XR1 |
| AXIN1 | ElF2S2 | KCNJ5 | PLCG1 | TBX3 |
| AXIN2 | ELF3 | KDM5C | POLE | TCEB1 |
| AZGP1 | EML4 | KDM6A | POT1 | TCF12 |
| B2M | EP300 | KDR | POU2AF1 | TCF7L2 |
| BAP1 | EPAS1 | KEAP1 | POU2F2 | TCP11L2 |
| BCLAF1 | EPHA2 | KEL | PPM1D | TDRD10 |
| BCOR | EPS8 | KIT | PPP2R1A | TERT |
| BHMT2 | ERBB2 | KLF4 | PPP6C | TET2 |
| BIRC3 | ERBB3 | KLF5 | PRDM1 | TG |
| BMPR2 | ERCC2 | KLHL8 | PRKAR1A | TGFBR2 |
| BRAF | ERG | KMT2A | PSG4 | TGIF1 |
| BRCA1 | ESR1 | KMT2B | PSIP1 | TIMM17A |
| BRCA2 | ETNK1 | KMT2C | PTCH1 | TNF |
| BRD7 | EZH2 | KMT2D | PTEN | TNFAIP3 |
| C3orf70 | FAM104A | KRAS | PTPN11 | TNFRSF14 |
| CACNA1D | FAM166A | KRT5 | PTPRB | TOP2A |
| CALR | FAM46C | LATS2 | QKI | TP53 |
| CARD11 | FAT1 | LCTL | RAC1 | TRAF3 |
| CASP8 | FBX011 | LZTR1 | RACGAP1 | TRAF7 |
| CBFB | FBXW7 | MAP2K1 | RAD21 | TRIM23 |
| CBL | FGFR1 | MAP2K2 | RASA1 | TSC1 |
| CBLB | FGFR2 | MAP2K4 | RB1 | TSC2 |
| CCDC120 | FGFR3 | MAP2K7 | RBM10 | TSHR |
| CCDC6 | FLG | MAP3K1 | RET | TTLL9 |
| CCND1 | FLT3 | MAX | RHEB | TYRO3 |
| CD1D | FOSL2 | MED12 | RHOA | U2AF1 |
| CD58 | FOXA1 | MED23 | RHOB | UBR5 |
| CD70 | FOXA2 | MEN1 | RIT1 | UPF3A |
| CD79A | FOXL2 | MET | RNF43 | VHL |
| CD79B | FOXQ1 | MGA | RPL10 | WASF3 |
| CDC27 | FRMD7 | MLH1 | RPL22 | WT1 |
| CDC73 | FUBP1 | MPL | RPL5 | XIRP2 |
| CDH1 | GAGE12J | MPO | RPS15 | XPO1 |
| CDH10 | GATA1 | MSH2 | RPS2 | ZBTB20 |
| CDK12 | GATA2 | MSH6 | RPS6KA3 | ZBTB7B |
| CDK4 | GATA3 | MTOR | RREB1 | ZFHX3 |
| CDKN1A | GNA11 | MUC17 | RUNX1 | ZFP36L1 |
| CDKN1B | GNA13 | MUC6 | RXRA | ZFP36L2 |
| CDKN2A | GNAQ | MXRA5 | SELP | ZFX |
| CDKN2C | GNAS | MYD88 | SETBP1 | ZMYM3 |
| CEBPA | GNB1 | MYOCD | SETD2 | ZNF471 |
| CHD4 | GNPTAB | MYOD1 | SF3B1 | ZNF620 |
| CHD8 | GPS2 | NBPF1 | SGK1 | ZNF750 |
| CIB3 | GTF2I | NCOR1 | SH2B3 | ZNF800 |
| CIC | GUSB | NF1 | SLC1A3 | ZNRF3 |
| CMTR2 | H3F3A | NF2 | SLC26A3 | ZRSR2 |
| CNBD1 | H3F3B | NFE2L2 | SLC44A3 | |

TABLE 3
Spatial computational tumor model parameters

| | | Default in | |
|---|---|---|---|
| Parameters | Description | basic model | Justifications/Remarks |
| N_{T} | Final tumor size | N_{T}~10⁹ cells for | There are ~10⁹ or more cells in a typical solid tumor. |
| | | both primary | |
| | | tumor and | |
| | | metastasis | |
| K | Deme size | K = | cells The demes recapitulate the glandular structure often found in colorectal |
| | | 5,000-10,000 | cancer in which the gland size is approximated at 2,000-10,000 cells ³. The |
| | | | deme size recapitulates the degree of spatial constraint and clone mixing |
| | | | during tumor growth. For instance, small deme size represents stringent |
| | | | spatial constraint and reduced subclone mixing, thereby hindering the efficacy |
| | | | of selection. In contrast, large deme size results in relaxed spatial constraint and |
| | | | enhanced subclone mixing. |
| p and q | The birth and death | p = 0.55 and | It has been reported that there is no significant growth rate difference in paired |
| | probability for each cell at | q = 0.45 | primary tumors and metastases ⁵. We therefore assume the same birth and death |
| | each generation during | | rates in primary tumor and metastasis. Given the choice of p and q values here, |
| | deme expansion, | | it takes about 3 years (assuming 4 days for each cell cycle) for the tumor to |
| | respectively | | grow from founder cell to diagnosis (~10⁹ cells) (FIG. 15b). |
| u | Passenger mutation rate | u = 0.3 | Mutation rate in normal somatic cells is at the order of 10⁻⁹ per base pair per |
| | per cell division in the | | cell division ⁹. Because of the genomic instability in many cancers, the per- |
| | ~60 Mb exonic regions | | cell division mutation rate for cancer is significantly higher than normal cells. |
| | | | We assume a mutation rate 5 × 10⁻⁹/base pair/division (equivalent to u = 0.3 |
| | | | per cell division for the 60M exonic region) in the simulations, giving rise |
| | | | to 20-200 subclonal SNVs (10% <CCF <60%) in each bulk sample in the |
| | | | simulations which is in consistent with the observed number in current study. |
| u_{b} | Mutation rate of beneficial | u_{b} = 10⁻⁵ | Bozic et al ⁷ estimated u_{b} to be at the order of 10⁻⁵ per cell division in |
| | driver mutations per cell | | the genome. |
| | division | | |
| s | Selection coefficient | s = 0.1 | We use relatively high selection s = 0.1, in order to robustly distinguish |
| | | | with the evolutionary dynamics of effectively neutral evolution ¹. |
| N_{d} | The primary tumor size | log10(N_{d})~ | We randomly chose 100 dissemination time points, correponding to the |
| | in cell number at the time | U(2, 9) | primary tumor size at the time of dissemination from a uniform distribution |
| | of dissemination | | log10(N_{d})~U(2, 9), each giving rise to an independent paired primary tumor |
| | | | and metastasis. |
| c | The number of cells from | c = 1 | We assume one single cell from a deme in tumor periphery seeds the |
| | primary tumor seeding a | | metastasis based on the pattern of commonly monoclonal seeding in the |
| | metastasis | | mCRC cohort. |

TABLE 4
Description of summary statistics for SCIMET

| Summary statistics | Descriptions |
|---|---|
| S ₁, S ₂ , S ₃ and S ₄ | The total number of primary-private sSNVs |
| | that are present at merged CCF >10%, |
| | 20%, 40% and 60%, respectively. |
| S ₅, S ₆, S ₇ and S ₈ | The total number of metastasis-private |
| | sSNVs that are present at merged |
| | CCF >10%, 20%, 40% and 60%, |
| | respectively. |
| S ₉ | The total number of sSNVs that are |
| | metastasis-clonal (merged CCF >60%) |
| | while primary-subclonal (10% < merged |
| | CCF <60%). |

TABLE 5
SCIMET Summary
statistics for the metastatic colorectal cancer cohort

| PM_pair | S1 | S2 | S3 | S4 | S5 | S6 | S7 | S8 | S9 |
|---|---|---|---|---|---|---|---|---|---|
| V402_BM | 106 | 29 | 9 | 6 | 24 | 21 | 20 | 20 | 2 |
| V824_BM | 108 | 23 | 4 | 2 | 39 | 29 | 26 | 25 | 3 |
| V953_BM | 295 | 190 | 64 | 33 | 54 | 33 | 21 | 21 | 2 |
| V974_BM | 66 | 59 | 49 | 45 | 35 | 30 | 30 | 30 | 1 |
| V930_LU | 32 | 12 | 7 | 2 | 88 | 78 | 48 | 33 | 2 |
| V930_BM | 29 | 11 | 6 | 2 | 52 | 48 | 47 | 47 | 1 |
| V750_BM | 63 | 26 | 6 | 2 | 98 | 42 | 19 | 17 | 11 |
| V46_BM | 20 | 19 | 12 | 11 | 58 | 54 | 51 | 45 | 4 |
| V514_BM | 16 | 16 | 16 | 9 | 42 | 27 | 26 | 24 | 2 |
| V559_LI | 18 | 15 | 13 | 11 | 103 | 68 | 13 | 6 | 5 |
| V559_BM | 13 | 13 | 11 | 8 | 66 | 65 | 34 | 26 | 4 |
| V855_BM | 32 | 32 | 21 | 14 | 21 | 21 | 14 | 12 | 2 |
| Uchi2__LI | 11 | 5 | 2 | 2 | 12 | 12 | 10 | 8 | 0 |
| Kim1_LI | 42 | 8 | 0 | 0 | 8 | 5 | 3 | 2 | 4 |
| Kim2_LI | 79 | 34 | 6 | 1 | 16 | 15 | 15 | 14 | 22 |
| Leung1__LI | 8 | 8 | 7 | 5 | 16 | 16 | 13 | 11 | 3 |
| Leung2__LI | 24 | 21 | 17 | 14 | 138 | 118 | 103 | 91 | 3 |
| Lim3_LI | 42 | 41 | 23 | 13 | 30 | 28 | 23 | 17 | 0 |
| Lim7_LI | 17 | 11 | 8 | 7 | 13 | 13 | 5 | 4 | 0 |
| Lim8_LI | 65 | 65 | 59 | 49 | 123 | 122 | 114 | 102 | 0 |
| Lim12_LI | 24 | 24 | 19 | 13 | 40 | 40 | 32 | 17 | 0 |
| Lim16_LI | 14 | 14 | 7 | 5 | 17 | 12 | 7 | 7 | 0 |
| Lim21_LI | 2 | 2 | 2 | 2 | 28 | 28 | 25 | 20 | 0 |

1. A method for determining an individual's risk for colorectal cancer, comprising:
examining genetic material of a biopsy of an individual having colorectal cancer;
detecting that the biopsy includes genetic aberrations occurring within the genes PTPRT, TCF7L2, AMER1 APC, KRAS, TP53, or SMAD4;
determining that each gene of one of the following combinations of gene sets exhibits a genetic abnormality that confers a pathogenic effect on gene function:
PTPRT and one of: APC, KRAS, TP53 or SMAD4,
PTPRT and APC and KRAS,
PRPRT and APC and TP53,
PTPRT and TP53 and KRAS,
PTPRT and TP53 and SMAD4,
PTPRT and TP53 and KRAS and SMAD4,
AMER1 and one of: APC, KRAS or TP53,
AMER1 and APC and KRAS,
AMER1 and APC and TP5,
TCF7L2 and one of: APC or TP53, or
TCF7L2 and APC and TP53.

2. The method as in claim 1, further comprising: administering to the individual a treatment based upon that each gene of a said gene set combination exhibits a genetic abnormality, which is further based upon the clinical stage of cancer progression.

3. The method as in claim 2, wherein the clinical stage is classified as Stage 0 and the treatment includes a local excision or a polypectomy and prolonged monitoring after the local excision or the polypectomy.

4. The method as in claim 2, wherein the clinical stage is classified as Stage I and the treatment includes a surgical resection and prolonged monitoring after the surgical resection.

5. The method as in claim 2, wherein the clinical stage is classified as Stage II and the treatment includes a surgical resection and an adjuvant chemotherapy.

6. The method as in claim 2, wherein the clinical stage is classified as Stage II and the treatment includes a surgical resection and a targeted therapy.

7. The method as in claim 2, wherein the clinical stage is classified as Stage III and the treatment includes a surgical resection with a prolonged adjuvant chemotherapy.

8. The method as in claim 2, wherein the clinical stage is classified as Stage III and the treatment includes a surgical resection and an adjuvant chemotherapy typical for metastatic colorectal cancer.

9. The method as in claim 2, wherein the clinical stage is classified as Stage III and the treatment includes a surgical resection and a targeted therapy.

10. The method as in claim 2, wherein the clinical stage is classified as Stage IV and the treatment includes an adjuvant chemotherapy and a targeted therapy.

11. The method as in claim 1, wherein the biopsy is a tumor biopsy or liquid biopsy.

12. The method as in claim 11, wherein the biopsy is derived from a primary tumor, a nodal tumor, or a distal tumor.

13. The method as in claim 1, wherein the genetic aberrations detected are single nucleotide variants, insertions, deletions, or copy number alterations (CNAs).

14. The method as in claim 1, wherein the determination that each gene of one of the following combinations of gene sets exhibits a genetic abnormality include analysis of at least one of: genomic sequence mutation, copy number aberration, DNA methylation, RNA transcript expression level, or protein expression level.

15. The method as in claim 1, wherein the genetic aberration is detected by an assay selected from the group consisting of:
nucleic acid hybridization, nucleic acid proliferation, and nucleic acid sequencing.

16. The method as in claim 1, wherein the pathogenic effect on the gene function is known to confer an oncogenic effect.

17. The method as in claim 1, wherein the pathogenic effect on the gene function is assumed to confer an oncogenic effect.

18. The method as in claim 1, wherein the pathogenic effect on the gene function is determined to likely confer an oncogenic effect.

19. The method as in claim 18, wherein the pathogenic effect on the gene function is determined by a computational program.

20. The method as in claim 18, wherein the pathogenic effect on the gene function is determined by a biological assay.

21.-43. (canceled)
